(19) United States
(12) Patent Application Publication
(10) Pub. No.: US 2014/0030259 A1
(43) Pub. Date: Jan. 30, 2014

(54) METHODS OF TREATING FGFR3 RELATED CONDITIONS

(71) Applicant: Genentech, Inc., South San Francisco, CA (US)

(72) Inventor: Dorothy French, San Carlos, CA (US)

(73) Assignee: Genentech, Inc., South San Francisco, CA (US)

(21) Appl. No.: 13/952,072

(22) Filed: Jul. 26, 2013

Related U.S. Application Data
(60) Provisional application No. 61/676,857, filed on Jul. 27, 2012.
(60) Provisional application No. 61/695,853, filed on Aug. 31, 2012.
(60) Provisional application No. 61/704,052, filed on Sep. 21, 2012.

(51) Int. Cl. A61K 39/395; G01N 33/574

(52) U.S. Cl. CPC A61K 39/3955; G01N 33/57484
USPC 424/133.1; 424/172.1; 424/143.1; 424/142.1; 435/7.23; 514/44 R

(57) ABSTRACT

Provided herein are biomarkers and therapies for the treatment of pathological conditions, such as cancer, and method of using FGFR3 antagonists. In particular, provided is FGFR3 as a biomarker for patient selection and prognosis in cancer, as well as methods of therapeutic treatment, articles of manufacture and methods for making them, diagnostic kits, methods of detection and methods of advertising related thereto.

### CROSS REFERENCE TO RELATED APPLICATIONS

This application claims the benefits of U.S. Provisional Patent Application No. 61/676,857, filed on Jul. 27, 2012, U.S. Provisional Patent Application No. 61/695,853, filed on Aug. 31, 2012, and U.S. Provisional Patent Application No. 61/704,052 filed on Sep. 21, 2012, which are hereby incorporated by reference in their entirety.

### SEQUENCE LISTING

The instant application contains a Sequence Listing which has been submitted in ASCII format via EFS-Web and is hereby incorporated by reference in its entirety. Said ASCII copy, created on Jul. 23, 2013, is named P4950R1US_Sequence_Listing.txt and is 49,775 bytes in size.

### FIELD

Provided herein are biomarkers and therapies for the treatment of pathological conditions, such as cancer, and method of using FGFR3 antagonists. In particular, provided is FGFR3 as a biomarker for patient selection and prognosis in cancer, as well as methods of therapeutic treatment, articles of manufacture and methods for making them, diagnostic kits, methods of detection and methods of advertising related thereto.

### BACKGROUND

Cancer remains to be one of the most deadly threats to human health. In the U.S., cancer affects nearly 1.3 million new patients each year, and is the second leading cause of death after heart disease, accounting for approximately 1 in 4 deaths. For example, breast cancer is the second most common form of cancer and the second leading cancer killer among American women. It is also predicted that cancer may surpass cardiovascular diseases as the number one cause of death within 5 years. Solid tumors are responsible for most of those deaths. Although there have been significant advances in the medical treatment of certain cancers, the overall 5-year survival rate for all cancers has improved only by about 10% in the past 20 years. Cancers, or malignant tumors, metastasize and grow rapidly in an uncontrolled manner, making timely detection and treatment extremely difficult.

Despite the significant advancement in the treatment of cancer, improved therapies are still being sought.

All references cited herein, including patent applications and publications, are incorporated by reference in their entirety.

### SUMMARY

Provided herein are FGFR3 antagonists (e.g., anti-FGFR3 antibodies) and methods of using the same. Provided are methods for treating an individual with disease or disorder comprising administering a therapeutically effective amount of a FGFR3 antagonist (e.g., anti-FGFR3 antibodies) to the individual if the individual has been found to have elevated levels of a FGFR3 biomarker.

Further provided herein are methods for treating a disease or disorder in an individual, the method comprising: determining that a sample from the individual comprises elevated levels of a FGFR3 biomarker, and administering an effective amount of a FGFR3 antagonist (e.g., anti-FGFR3 antibodies) to the individual, whereby the disease or disorder is treated.

Provided herein are methods of treating a disease or disorder in an individual comprising administering to the individual an effective amount of an FGFR3 antagonist (e.g., anti-FGFR3 antibodies), wherein treatment is based upon elevated levels of a FGFR3 biomarker in a sample from the individual.

In addition, provided herein are methods for selecting a therapy for an individual with a disease or disorder comprising determining levels of a FGFR3 biomarker, and selecting a medicament based on the levels of the biomarker. In some embodiments, the medicament is selected based upon elevated levels of the FGFR3 biomarker.

Provided herein are methods of identifying an individual with a disease or disorder who is more or less likely to exhibit benefit from treatment comprising a FGFR3 antagonist (e.g., anti-FGFR3 antibodies), the method comprising: determining levels of a FGFR3 biomarker in a sample from the individual, wherein elevated levels of the FGFR3 biomarker in the sample indicates that the individual is more likely to exhibit benefit from treatment comprising the FGFR3 antagonist (e.g., anti-FGFR3 antibodies) or a reduced levels of the FGFR3 biomarker indicates that the individual is less likely to exhibit benefit from treatment comprising the FGFR3 antagonist (e.g., anti-FGFR3 antibodies).

Further provided herein are methods for advertising a FGFR3 antagonist (e.g., anti-FGFR3 antibodies) comprising promoting, to a target audience, the use of the FGFR3 antagonist (e.g., anti-FGFR3 antibodies) for treating an individual with a disease or disorder based on levels of a FGFR3 biomarker. In some embodiments, the use of the FGFR3 antagonist is based upon elevated levels of the FGFR3 biomarker.

Provided herein are also assays for identifying an individual with a disease or disorder to receive a FGFR3 antagonist (e.g., anti-FGFR3 antibodies), the method comprising: (a) determining levels of a FGFR3 biomarker in a sample from the individual; (b) recommending a FGFR3 antagonist (e.g., anti-FGFR3 antibodies) based upon the levels of the FGFR3 biomarker. In some embodiments, the FGFR3 antagonist is recommended based upon elevated levels of the FGFR3 biomarker.

Provided herein are diagnostic kits comprising one or more reagent for determining levels of a FGFR3 biomarker in a sample from an individual with a disease or disorder, wherein detection of elevated levels of the FGFR3 biomarker means increased efficacy when the individual is treated with a FGFR3 antagonist (e.g., anti-FGFR3 antibodies), and wherein detection of a low or substantially undetectable levels of a FGFR3 biomarker means a decreased efficacy when the individual with the disease is treated with the FGFR3 antagonist (e.g., anti-FGFR3 antibodies). Provided herein are also articles of manufacture comprising, packaged together, a FGFR3 antagonist (e.g., anti-FGFR3 antibodies) in a pharmaceutically acceptable carrier and a package insert indicating that the FGFR3 antagonist (e.g., anti-FGFR3 antibodies) is for treating a patient with a disease or disorder based on expression of a FGFR3 biomarker. Treatment methods include any of the treatment methods disclosed herein. Further provided are the invention concerns a method for manufacturing an article of manufacture comprising combining in a package a pharmaceutical composition comprising a FGFR3 antagonist (e.g., anti-FGFR3 antibodies) and a package insert indicating that the pharmaceutical composition is for treating a patient with a disease or disorder based on expression of FGFR3 biomarker.

In some embodiments of any of the methods, assays and/or kits, the FGFR3 biomarker is FGFR3. In some embodiments, FGFR3 is detected by immunohistochemistry. In some embodiments, elevated expression of a FGFR3 biomarker in a sample from an individual is elevated protein expression and, in further embodiments, is determined using IHC. In some embodiments, elevated levels of a FGFR3 biomarker is detected by IHC clinical diagnosis of positive or IHC clinical score of 1 or higher. In some embodiments, the IHC clinical score of 1 or higher is 2 or higher. In some embodiments, the IHC clinical score of 1 or higher is 3. In some embodiments, the IHC clinical score is 3. In some embodiments, the IHC clinical score is 2 or 3. In some embodiments, an IHC clinical score of 1 represents a) >10% cytoplasmic and/or membrane staining and b) weak cytoplasmic and/or membrane staining with moderate and/or strong staining being <10% of positively stained cells. In some embodiments, an IHC clinical score of 1 represents staining similar to and/or substantially the same as RPMI8226 cell line staining. In some embodiments, an IHC clinical score of 2 represents a) >10% cytoplasmic and/or membrane staining and b) moderate cytoplasmic and/or membrane staining in >10% of cells, with strong staining being <10% of positively stained cells; weak staining may or may not be present. In some embodiments, an IHC clinical score of 2 represents staining similar to and/or substantially the same as OPM2 cell line staining. In some embodiments, an IHC clinical score of 3 represents a) >10% cytoplasmic and/or membrane staining and b) strong cytoplasmic and/or membrane staining in >10% of positively staining cells; weak and moderate staining may or may not be present. In some embodiments, an IHC clinical score of 3 represents staining similar to and/or substantially the same as KMS11 cell line staining.

In some embodiments, FGFR3 is detected by immunohistochemistry using an anti-FGFR3 diagnostic antibody. In some embodiments, the FGFR3 diagnostic antibody specifically binds human FGFR3. In some embodiments, the FGFR3 diagnostic antibody specifically binds an epitope comprising amino acids 25-124 of human FGFR3. In some embodiments, the FGFR3 diagnostic antibody specifically binds an epitope comprising LGTEQRVVGRAAEVPGPEPGQQEQLVFGSGDAVELSCPPPGGGPMGP TVWVKDGTGLVPSERVLVGPQRLQVLNASHEDSGAYSCRQRLTQRVLCHFSVR (SEQ ID NO: 181). In some embodiments of any of the FGFR3 diagnostic antibodies, the FGFR3 diagnostic antibody is a rat, mouse, or rabbit antibody. In some embodiments of any of the FGFR3 diagnostic antibodies, the FGFR3 diagnostic antibody is a monoclonal antibody. In some embodiments of any of the FGFR3 diagnostic antibodies, the FGFR3 diagnostic antibody is an IgG2 antibody. In some embodiments of any of the FGFR3 diagnostic antibodies, the FGFR3 diagnostic antibody is an IgG2a antibody. In some embodiments of any of the FGFR3 diagnostic antibodies, the FGFR3 diagnostic antibody is sc-13121 (i.e., B-9) from Santa Cruz Biotechnology.

In some embodiments of any of the methods, assays and/or kits, the FGFR3 biomarker is FGFR3 mutation. In some embodiments, the FGFR3 mutation is encodes for one or more of the following FGFR3 amino acid variants: FGFR3 R248C, FGFR3 S249C, FGFR3 G370C, FGFR3 S371C, FGFR3 Y373C, FGFR3 G380R, FGFR3 K650X (e.g., FGFR3 K650E), FGFR3 K650M, and FGFR3 G697C. In some embodiments, the FGFR3 mutation is one or more of the following FGFR3 amino acid variants: FGFR3 c.746C>G, FGFR3 c.1118A>G, FGFR3 c.742C>T, FGFR3c.1108G>T, FGFR3 c.1111A>T.

In some embodiments of any of the methods, assays and/or kits, the sample is a tissue sample from the individual. In some embodiments, the tissue sample is a tumor tissue sample (e.g., biopsy tissue). In some embodiments, the tissue sample is bladder tissue. In some embodiments, the tissue sample is urothelial tissue. In some embodiments, the tissue sample is tissue adjacent the bladder.

In some embodiments of any of the methods, assays and/or kits, the methods, assays and/or kits further comprises administering an effective amount of the FGFR3 antagonist to the individual.

In some embodiments of any of the methods, assays and/or kits, the FGFR3 antagonist is an antibody, binding polypeptide, small molecule, and/or polynucleotide. In some embodiments, the FGFR3 antagonist is an anti-FGFR3 antibody. In some embodiments, the antibody is a monoclonal antibody. In some embodiments, the antibody is a human, humanized, or chimeric antibody.

In some embodiments of any of the methods, assays and/or kits, the disease or disorder is a proliferative disease or disorder. In some embodiments, the proliferative disease or disorder is cancer. In some embodiments, the cancer is a solid tumor. In some embodiments, the cancer is bladder cancer. In some embodiments, the cancer is transitional cell carcinoma (i.e., urothelial cell carcinoma).

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 | Schematic of anti-FGFR3 antibody IHC staining protocol.

FIG. 2 | A-B) Negative IHC staining of tissue samples using anti-FGFR3 antibody (sc-13121; B-9 from Santa Cruz Biotechnology). C) IHC staining of H1155 cell line using anti-FGFR3 antibody (sc-13121; B-9 from Santa Cruz Biotechnology).

FIG. 3 | A-D) IHC staining of tissue samples using anti-FGFR3 antibody (sc-13121; B-9 from Santa Cruz Biotechnology) with a clinical score of 1. E) IHC staining of RPMI8226 cell line using anti-FGFR3 antibody (sc-13121; B-9 from Santa Cruz Biotechnology).

FIG. 4 | A-D) IHC staining of tissue samples using anti-FGFR3 antibody (sc-13121; B-9 from Santa Cruz Biotechnology) with a clinical score of 2. E) IHC staining of OPM2 cell line using anti-FGFR3 antibody (sc-13121; B-9 from Santa Cruz Biotechnology).

FIG. 5 | A-D) IHC staining of tissue samples using anti-FGFR3 antibody (sc-13121; B-9 from Santa Cruz Biotechnology) with a clinical score of 3. E) IHC staining of KSM11 cell line using anti-FGFR3 antibody (sc-13121; B-9 from Santa Cruz Biotechnology).

FIG. 6 | A-E) IHC staining of tissue samples using anti-FGFR3 antibody (sc-13121; B-9 from Santa Cruz Biotechnology).

FIG. 7 | A-C) IHC staining of panel of urothelial carcinoma tissue using anti-FGFR3 antibody (sc-13121; B-9 from Santa Cruz Biotechnology).

FIG. 8 | A-C) IHC staining of panel of clinical tissue samples (A: Patient 4, B: Patient 8, and C: Patient 9) using anti-FGFR3 antibody (sc-13121; B-9 from Santa Cruz Biotechnology).

FIG. 9 | A-F: Heavy chain and light chain HVR loop sequences of anti-FGFR3 antibodies. The figures show the heavy chain HVR sequences, H1, H2, and H3, and light chain HVR sequences, L1, L2, and L3. Sequence numbering is as follows:

Clone 184.6 (HVR-H1 is SEQ ID NO: 1; HVR-H2 is SEQ ID NO:2; HVR-H3 is SEQ ID NO:3; HVR-L1 is SEQ ID NO:4; HVR-L2 is SEQ ID NO:5; HVR-L3 is SEQ ID NO:6);

Clone 184.6.1 (HVR-H1 is SEQ ID NO:7; HVR-H2 is SEQ ID NO:8; HVR-H3 is SEQ ID NO:9; HVR-L1 is SEQ ID NO:10; HVR-L2 is SEQ ID NO:11; HVR-L3 is SEQ ID NO: 12)

Clone 184.6.58 (HVR-H1 is SEQ ID NO:13; HVR-H2 is SEQ ID NO:14; HVR-H3 is SEQ ID NO:15; HVR-L1 is SEQ ID NO:16; HVR-L2 is SEQ ID NO:17; HVR-L3 is SEQ ID NO:18)

Clone 184.6.62 (HVR-H1 is SEQ ID NO:48; HVR-H2 is SEQ ID NO:49; HVR-H3 is SEQ ID NO:50; HVR-L1 is SEQ ID NO:51; HVR-L2 is SEQ ID NO:52; HVR-L3 is SEQ ID NO:53)

Clone 184.6.21 (HVR-H1 is SEQ ID NO:54; HVR-H2 is SEQ ID NO:55; HVR-H3 is SEQ ID NO:56; HVR-L1 is SEQ ID NO:57; HVR-L2 is SEQ ID NO:58; HVR-L3 is SEQ ID NO:59)

Clone 184.6.49 (HVR-H1 is SEQ ID NO:60; HVR-H2 is SEQ ID NO:61; HVR-H3 is SEQ ID NO:62; HVR-L1 is SEQ ID NO:63; HVR-L2 is SEQ ID NO:64; HVR-L3 is SEQ ID NO:65)

Clone 184.6.51 (HVR-H1 is SEQ ID NO:66; HVR-H2 is SEQ ID NO:67; HVR-H3 is SEQ ID NO:68; HVR-L1 is SEQ ID NO:69; HVR-L2 is SEQ ID NO:70; HVR-L3 is SEQ ID NO:71)

Clone 184.6.52 (HVR-H1 is SEQ ID NO:72; HVR-H2 is SEQ ID NO:73; HVR-H3 is SEQ ID NO:74; HVR-L1 is SEQ ID NO:75; HVR-L2 is SEQ ID NO:76; HVR-L3 is SEQ ID NO:77)

Clone 184.6.92 (HVR-H1 is SEQ ID NO:78; HVR-H2 is SEQ ID NO:79; HVR-H3 is SEQ ID NO:80; HVR-L1 is SEQ ID NO:81; HVR-L2 is SEQ ID NO:82; HVR-L3 is SEQ ID NO:83)

Clone 184.6.1.N54S (HVR-H1 is SEQ ID NO:84; HVR-H2 is SEQ ID NO:85; HVR-H3 is SEQ ID NO:86; HVR-L1 is SEQ ID NO:87; HVR-L2 is SEQ ID NO:88; HVR-L3 is SEQ ID NO:89)

Clone 184.6.1.N54G (HVR-H1 is SEQ ID NO:90; HVR-H2 is SEQ ID NO:91; HVR-H3 is SEQ ID NO:92; HVR-L1 is SEQ ID NO:93; HVR-L2 is SEQ ID NO:94; HVR-L3 is SEQ ID NO:95)

Clone 184.6.1.N54A (HVR-H1 is SEQ ID NO:96; HVR-H2 is SEQ ID NO:97; HVR-H3 is SEQ ID NO:98; HVR-L1 is SEQ ID NO:99; HVR-L2 is SEQ ID NO:100; HVR-L3 is SEQ ID NO:101)

Clone 184.6.1.N54Q (HVR-H1 is SEQ ID NO:102; HVR-H2 is SEQ ID NO:103; HVR-H3 is SEQ ID NO:104; HVR-L1 is SEQ ID NO:105; HVR-L2 is SEQ ID NO:106; HVR-L3 is SEQ ID NO: 107)

Clone 184.6.58.N54S (HVR-H1 is SEQ ID NO:108; HVR-H2 is SEQ ID NO:109; HVR-H3 is SEQ ID NO:110; HVR-L1 is SEQ ID NO:111; HVR-L2 is SEQ ID NO:112; HVR-L3 is SEQ ID NO:113)

Clone 184.6.58.N54G (HVR-H1 is SEQ ID NO:114; HVR-H2 is SEQ ID NO:115; HVR-H3 is SEQ ID NO:116; HVR-L1 is SEQ ID NO:117; HVR-L2 is SEQ ID NO:118; HVR-L3 is SEQ ID NO:119)

Clone 184.6.58.N54A (HVR-H1 is SEQ ID NO:120; HVR-H2 is SEQ ID NO:121; HVR-H3 is SEQ ID NO:122; HVR-L1 is SEQ ID NO:123; HVR-L2 is SEQ ID NO:124; HVR-L3 is SEQ ID NO: 125)

Clone 184.6.58.N54Q (HVR-H1 is SEQ ID NO:126; HVR-H2 is SEQ ID NO:127; HVR-H3 is SEQ ID NO:128; HVR-L1 is SEQ ID NO:129; HVR-L2 is SEQ ID NO:130; HVR-L3 is SEQ ID NO:131).

Clone 184.6.1.NS D30E (HVR-H1 is SEQ ID NO:143; HVR-H2 is SEQ ID NO:144; HVR-H3 is SEQ ID NO:145; HVR-L1 is SEQ ID NO:140; HVR-L2 is SEQ ID NO:141; HVR-L3 is SEQ ID NO: 142).

Amino acid positions are numbered according to the Kabat numbering system as described below.

FIG. 10 | Depict the amino acid sequences of the heavy chain variable regions and light chain variable regions of anti-FGFR3 antibodies 184.6.1.N54S, 184.6.1′, 184.6.58, and 184.6.62.

### DETAILED DESCRIPTION

### I. Definitions

The terms “Fibroblast Growth Factor Receptor 3” and “FGFR3” refer herein to a native sequence FGFR3 polypeptide, polypeptide variants and fragments of a native sequence polypeptide and polypeptide variants (which are further defined herein). The FGFR3 polypeptide described herein may be that which is isolated from a variety of sources, such as from human tissue types or from another source, or prepared by recombinant or synthetic methods.

A “native sequence FGFR3 polypeptide” comprises a polypeptide having the same amino acid sequence as the corresponding FGFR3 polypeptide derived from nature. In one embodiment, a native sequence FGFR3 polypeptide comprises the amino acid sequence from UniProt database of P22607-1 or P22607-2.

“FGFR3 polypeptide variant”, or variations thereof, means a FGFR3 polypeptide, generally an active FGFR3 polypeptide, as defined herein having at least about 80% amino acid sequence identity with any of the native sequence FGFR3 polypeptide sequences as disclosed herein. Such FGFR3 polypeptide variants include, for instance, FGFR3 polypeptides wherein one or more amino acid residues are added, or deleted, at the N- or C-terminus of a native amino acid sequence. Ordinarily, a FGFR3 polypeptide variant will have at least about 80% amino acid sequence identity, alternatively at least about 81%, 82%, 83%, 84%, 85%, 86%, 87%, 88%, 89%, 90%, 91%, 92%, 93%, 94%, 95%, 96%, 97%, 98%, or 99% amino acid sequence identity, to a native sequence FGFR3 polypeptide sequence as disclosed herein. Ordinarily, FGFR3 variant polypeptides are at least about 10 amino acids in length, alternatively at least about 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600 amino acids in length, or more. Optionally, FGFR3 variant polypeptides will have no more than one conservative amino acid substitution as compared to a native FGFR3 polypeptide sequence, alternatively no more than 2, 3, 4, 5, 6, 7, 8, 9, or 10 conservative amino acid substitution as compared to the native FGFR3 polypeptide sequence.

The term “FGFR3 antagonist” as defined herein is any molecule that partially or fully blocks, inhibits, or neutralizes a biological activity mediated by a native sequence FGFR3. In certain embodiments such antagonist binds to FGFR3. According to one embodiment, the antagonist is a polypeptide. According to another embodiment, the antagonist is an anti-FGFR3 antibody. According to another embodiment, the antagonist is a small molecule antagonist. According to another embodiment, the antagonist is a polynucleotide antagonist.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), “(O)NR₂ (“amidate”), P(O)R, P(O)OR, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

“Oligonucleotide,” as used herein, generally refers to short, single stranded, polynucleotides that are, but not necessarily, less than about 250 nucleotides in length. Oligonucleotides may be synthetic. The terms “oligonucleotide” and “polynucleotide” are not mutually exclusive. The description above for polynucleotides is equally and fully applicable to oligonucleotides.

The term “primer” refers to a single stranded polynucleotide that is capable of hybridizing to a nucleic acid and following polymerization of a complementary nucleic acid, generally by providing a free 3′-OH group.

The term “small molecule” refers to any molecule with a molecular weight of about 2000 daltons or less, preferably of about 500 daltons or less.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

The terms “anti-FGFR3 antibody” and “an antibody that binds to FGFR3” refer to an antibody that is capable of binding FGFR3 with sufficient affinity such that the antibody is useful as a diagnostic and/or therapeutic agent in targeting FGFR3. In one embodiment, the extent of binding of an anti-FGFR3 antibody to an unrelated, non-FGFR3 protein is less than about 10% of the binding of the antibody to FGFR3 as measured, e.g., by a radioimmunoassay (RIA). In certain embodiments, an anti-FGFR3 antibody binds to an epitope of FGFR3 that is conserved among FGFR3 from different species.

A “blocking” antibody or an “antagonist” antibody is one which inhibits or reduces biological activity of the antigen it binds. Preferred blocking antibodies or antagonist antibodies substantially or completely inhibit the biological activity of the antigen.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g., scFv); and multispecific antibodies formed from antibody fragments.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

The terms “full length antibody,” “intact antibody,” and “whole antibody” are used herein interchangeably to refer to an antibody having a structure substantially similar to a native antibody structure or having heavy chains that contain an Fc region as defined herein.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

An “immunoconjugate” is an antibody conjugated to one or more heterologous molecule(s), including but not limited to a cytotoxic agent.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “detection” includes any means of detecting, including direct and indirect detection.

The term “biomarker” as used herein refers to an indicator, e.g., predictive, diagnostic, and/or prognostic, which can be detected in a sample. The biomarker may serve as an indicator of a particular subtype of a disease or disorder (e.g., cancer) characterized by certain, molecular, pathological, histological, and/or clinical features. In some embodiments, a biomarker is a gene. Biomarkers include, but are not limited to, polynucleotides (e.g., DNA, and/or RNA), polypeptides, polypeptide and polynucleotide modifications (e.g. posttranslational modifications), carbohydrates, and/or glycolipid-based molecular markers.

The terms “biomarker signature,” “signature,” “biomarker expression signature,” or “expression signature” are used interchangeably herein and refer to one or a combination of biomarkers whose expression is an indicator, e.g., predictive, diagnostic, and/or prognostic. The biomarker signature may serve as an indicator of a particular subtype of a disease or disorder (e.g., cancer) characterized by certain molecular, pathological, histological, and/or clinical features. In some embodiments, the biomarker signature is a “gene signature.” The term “gene signature” is used interchangeably with “gene expression signature” and refers to one or a combination of polynucleotides whose expression is an indicator, e.g., predictive, diagnostic, and/or prognostic. In some embodiments, the biomarker signature is a “protein signature.” The term “protein signature” is used interchangeably with “protein expression signature” and refers to one or a combination of polypeptides whose expression is an indicator, e.g., predictive, diagnostic, and/or prognostic.

The “amount” or “level” of a biomarker associated with an increased clinical benefit to an individual is a detectable level in a biological sample. These can be measured by methods known to one skilled in the art and also disclosed herein. The expression level or amount of biomarker assessed can be used to determine the response to the treatment.

The terms “level of expression” or “expression level” in general are used interchangeably and generally refer to the amount of a biomarker in a biological sample. “Expression” generally refers to the process by which information (e.g., gene-encoded and/or epigenetic) is converted into the structures present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., posttranslational modification of a polypeptide) shall also be regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, transfer and ribosomal RNAs).

“Elevated expression,” “elevated expression levels,” or “elevated levels” refers to an increased expression or increased levels of a biomarker in an individual relative to a control, such as an individual or individuals who are not suffering from the disease or disorder (e.g., cancer) or an internal control (e.g., housekeeping biomarker).

“Reduced expression,” “reduced expression levels,” or “reduced levels” refers to a decrease expression or decreased levels of a biomarker in an individual relative to a control, such as an individual or individuals who are not suffering from the disease or disorder (e.g., cancer) or an internal control (e.g., housekeeping biomarker). In some embodiments, reduced expression is little or no expression.

The term “housekeeping biomarker” refers to a biomarker or group of biomarkers (e.g., polynucleotides and/or polypeptides) which are typically similarly present in all cell types. In some embodiments, the housekeeping biomarker is a “housekeeping gene.” A “housekeeping gene” refers herein to a gene or group of genes which encode proteins whose activities are essential for the maintenance of cell function and which are typically similarly present in all cell types.

“Amplification,” as used herein generally refers to the process of producing multiple copies of a desired sequence. “Multiple copies” mean at least two copies. A “copy” does not necessarily mean perfect sequence complementarity or identity to the template sequence. For example, copies can include nucleotide analogs such as deoxyinosine, intentional sequence alterations (such as sequence alterations introduced through a primer comprising a sequence that is hybridizable, but not complementary, to the template), and/or sequence errors that occur during amplification.

The term “multiplex-PCR” refers to a single PCR reaction carried out on nucleic acid obtained from a single source (e.g., an individual) using more than one primer set for the purpose of amplifying two or more DNA sequences in a single reaction.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, can be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) overnight hybridization in a solution that employs 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with a 10 minute wash at 42° C. in 0.2×SSC (sodium chloride/sodium citrate) followed by a 10 minute high-stringency wash consisting of 0.1×SSC containing EDTA at 55° C.

“Moderately stringent conditions” can be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

The term “diagnosis” is used herein to refer to the identification or classification of a molecular or pathological state, disease or condition (e.g., cancer). For example, “diagnosis” may refer to identification of a particular type of cancer. “Diagnosis” may also refer to the classification of a particular subtype of cancer, e.g., by histopathological criteria, or by molecular features (e.g., a subtype characterized by expression of one or a combination of biomarkers (e.g., particular genes or proteins encoded by said genes)).

The term “aiding diagnosis” is used herein to refer to methods that assist in making a clinical determination regarding the presence, or nature, of a particular type of symptom or condition of a disease or disorder (e.g., cancer). For example, a method of aiding diagnosis of a disease or condition (e.g., cancer) can comprise measuring certain biomarkers in a biological sample from an individual.

The term “sample,” as used herein, refers to a composition that is obtained or derived from a subject and/or individual of interest that contains a cellular and/or other molecular entity that is to be characterized and/or identified, for example based on physical, biochemical, chemical and/or physiological characteristics. For example, the phrase “disease sample” and variations thereof refers to any sample obtained from a subject of interest that would be expected or is known to contain the cellular and/or molecular entity that is to be characterized. Samples include, but are not limited to, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, and combinations thereof.

By “tissue sample” or “cell sample” is meant a collection of similar cells obtained from a tissue of a subject or individual. The source of the tissue or cell sample may be solid tissue as from a fresh, frozen and/or preserved organ, tissue sample, biopsy, and/or aspirate; blood or any blood constituents such as plasma; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. The tissue sample may contain compounds which are not naturally intermixed with the tissue in nature such as preservatives, anticoagulants, buffers, fixatives, nutrients, antibiotics, or the like.

A “reference sample”, “reference cell”, “reference tissue”, “control sample”, “control cell”, or “control tissue”, as used herein, refers to a sample, cell, tissue, standard, or level that is used for comparison purposes. In one embodiment, a reference sample, reference cell, reference tissue, control sample, control cell, or control tissue is obtained from a healthy and/or non-diseased part of the body (e.g., tissue or cells) of the same subject or individual. For example, healthy and/or non-diseased cells or tissue adjacent to the diseased cells or tissue (e.g., cells or tissue adjacent to a tumor). In another embodiment, a reference sample is obtained from an untreated tissue and/or cell of the body of the same subject or individual. In yet another embodiment, a reference sample, reference cell, reference tissue, control sample, control cell, or control tissue is obtained from a healthy and/or non-diseased part of the body (e.g., tissues or cells) of an individual who is not the subject or individual. In even another embodiment, a reference sample, reference cell, reference tissue, control sample, control cell, or control tissue is obtained from an untreated tissue and/or cell of the body of an individual who is not the subject or individual.

For the purposes herein a “section” of a tissue sample is meant a single part or piece of a tissue sample, e.g. a thin slice of tissue or cells cut from a tissue sample. It is understood that multiple sections of tissue samples may be taken and subjected to analysis, provided that it is understood that the same section of tissue sample may be analyzed at both morphological and molecular levels, or analyzed with respect to both polypeptides and polynucleotides.

By “correlate” or “correlating” is meant comparing, in any way, the performance and/or results of a first analysis or protocol with the performance and/or results of a second analysis or protocol. For example, one may use the results of a first analysis or protocol in carrying out a second protocols and/or one may use the results of a first analysis or protocol to determine whether a second analysis or protocol should be performed. With respect to the embodiment of polynucleotide analysis or protocol, one may use the results of the polynucleotide expression analysis or protocol to determine whether a specific therapeutic regimen should be performed.

“Individual response” or “response” can be assessed using any endPoint indicating a benefit to the individual, including, without limitation, (1) inhibition, to some extent, of disease progression (e.g., cancer progression), including slowing down and complete arrest; (2) a reduction in tumor size; (3) inhibition (i.e., reduction, slowing down or complete stopping) of cancer cell infiltration into adjacent peripheral organs and/or tissues; (4) inhibition (i.e. reduction, slowing down or complete stopping) of metasisis; (5) relief, to some extent, of one or more symptoms associated with the disease or disorder (e.g., cancer); (6) increase in the length of progression free survival; and/or (9) decreased mortality at a given Point of time following treatment.

The term “substantially the same,” as used herein, denotes a sufficiently high degree of similarity between two numeric values, such that one of skill in the art would consider the difference between the two values to be of little or no biological and/or statistical significance within the context of the biological characteristic measured by said values (e.g., Kd values or expression). The difference between said two values is, for example, less than about 50%, less than about 40%, less than about 30%, less than about 20%, and/or less than about 10% as a function of the reference/comparator value.

The phrase “substantially different,” as used herein, denotes a sufficiently high degree of difference between two numeric values such that one of skill in the art would consider the difference between the two values to be of statistical significance within the context of the biological characteristic measured by said values (e.g., Kd values). The difference between said two values is, for example, greater than about 10%, greater than about 20%, greater than about 30%, greater than about 40%, and/or greater than about 50% as a function of the value for the reference/comparator molecule.

The word “label” when used herein refers to a detectable compound or composition. The label is typically conjugated or fused directly or indirectly to a reagent, such as a polynucleotide probe or an antibody, and facilitates detection of the reagent to which it is conjugated or fused. The label may itself be detectable (e.g., radioisotope labels or fluorescent labels) or, in the case of an enzymatic label, may catalyze chemical alteration of a substrate compound or composition which results in a detectable product.

An “effective amount” of an agent refers to an amount effective, at dosages and for periods of time necessary, to achieve the desired therapeutic or prophylactic result.

A “therapeutically effective amount” of a substance/molecule, agonist or antagonist may vary according to factors such as the disease state, age, sex, and weight of the individual, and the ability of the substance/molecule, agonist or antagonist to elicit a desired response in the individual. A therapeutically effective amount is also one in which any toxic or detrimental effects of the substance/molecule, agonist or antagonist are outweighed by the therapeutically beneficial effects. A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies are used to delay development of a disease or to slow the progression of a disease.

The term “anti-cancer therapy” refers to a therapy useful in treating cancer. Examples of anti-cancer therapeutic agents include, but are limited to, e.g., chemotherapeutic agents, growth inhibitory agents, cytotoxic agents, agents used in radiation therapy, anti-angiogenesis agents, apoptotic agents, anti-tubulin agents, and other agents to treat cancer, anti-CD20 antibodies, platelet derived growth factor inhibitors (e.g., Gleevec™ (Imatinib Mesylate)), a COX-2 inhibitor (e.g., celecoxib), interferons, cytokines, antagonists (e.g., neutralizing antibodies) that bind to one or more of the following targets PDGFR-beta, BlyS, APRIL, BCMA receptor(s), TRAIL/Apo2, and other bioactive and organic chemical agents, etc. Combinations thereof are also included in the invention.

The term “cytotoxic agent” as used herein refers to a substance that inhibits or prevents the function of cells and/or causes destruction of cells. The term is intended to include radioactive isotopes (e.g., At²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents e.g., methotrexate, adriamicin, vinca alkaloids (vincristine, vinblastine, etoposide), doxorubicin, melphalan, mitomycin C, chlorambucil, daunorubicin or other intercalating agents, enzymes and fragments thereof such as nucleolytic enzymes, antibiotics, and toxins such as small molecule toxins or enzymatically active toxins of bacterial, fungal, plant or animal origin, including fragments and/or variants thereof, and the various antitumor or anticancer agents disclosed below. Other cytotoxic agents are described below. A tumoricidal agent causes destruction of tumor cells.

A “chemotherapeutic agent” refers to a chemical compound useful in the treatment of cancer. Examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Nicolaou et al., Angew. Chem. Intl. Ed. Engl., 33: 183-186 (1994)); CDP323, an oral alpha-4 integrin inhibitor; dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®), liposomal doxorubicin TLC D-99 (MYOCET®), peglylated liposomal doxorubicin (CAELYX®), and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2′-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoid, e.g., paclitaxel (TAXOL®), albumin-engineered nanoparticle formulation of paclitaxel (ABRAXANE™), and docetaxel (TAXOTERE®); chloranbucil; 6-thioguanine; mercaptopurine; methotrexate; platinum agents such as cisplatin, oxaliplatin (e.g., ELOXATIN®), and carboplatin; vincas, which prevent tubulin polymerization from forming microtubules, including vinblastine (VELBAN®), vincristine (ONCOVIN®), vindesine (ELDISINE®, FILDESIN®), and vinorelbine (NAVELBINE®); etoposide (VP-16); ifosfamide; mitoxantrone; leucovorin; novantrone; edatrexate; daunomycin; aminopterin; ibandronate; to FGFR3somerase inhibitor RFS 2000; difluoromethylornithine (DMFO); retinoids such as retinoic acid, including bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; to FGFR3somerase 1 inhibitor (e.g., LURTOTECAN®); rmRH (e.g., ABARELIX®); BAY439006 (sorafenib; Bayer); SU-11248 (sunitinib, SUTENT®, Pfizer); perifosine, COX-2 inhibitor (e.g., celecoxib or etoricoxib), proteosome inhibitor (e.g., PS341); bortezomib (VELCADE®); CCI-779; tipifarnib (R11577); orafenib, ABT510; Bcl-2 inhibitor such as oblimersen sodium (GENASENSE®); pixantrone; EGFR inhibitors (see definition below); tyrosine kinase inhibitors (see definition below); serine-threonine kinase inhibitors such as rapamycin (sirolimus, RAPAMUNE®); farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Chemotherapeutic agents as defined herein include “anti-hormonal agents” or “endocrine therapeutics” which act to regulate, reduce, block, or inhibit the effects of hormones that can promote the growth of cancer. They may be hormones themselves, including, but not limited to: anti-estrogens with mixed agonist/antagonist profile, including, tamoxifen (NOLVADEX®), 4-hydroxytamoxifen, toremifene (FARESTON®), idoxifene, droloxifene, raloxifene (EVISTA®), trioxifene, keoxifene, and selective estrogen receptor modulators (SERMs) such as SERM3; pure anti-estrogens without agonist properties, such as fulvestrant (FASLODEX®), and EM800 (such agents may block estrogen receptor (ER) dimerization, inhibit DNA binding, increase ER turnover, and/or suppress ER levels); aromatase inhibitors, including steroidal aromatase inhibitors such as formestane and exemestane (AROMASIN®), and nonsteroidal aromatase inhibitors such as anastrazole (ARIMIDEX®), letrozole (FEMARA®) and aminoglutethimide, and other aromatase inhibitors include vorozole (RIVISOR®), megestrol acetate (MEGASE®), fadrozole, and 4(5)-imidazoles; lutenizing hormone-releaseing hormone agonists, including leuprolide (LUPRON® and ELIGARD®), goserelin, buserelin, and tripterelin; sex steroids, including progestines such as megestrol acetate and medroxyprogesterone acetate, estrogens such as diethylstilbestrol and premarin, and androgens/retinoids such as fluoxymesterone, all transretionic acid and fenretinide; onapristone; anti-progesterones; estrogen receptor down-regulators (ERDs); anti-androgens such as flutamide, nilutamide and bicalutamide; and pharmaceutically acceptable salts, acids or derivatives of any of the above; as well as combinations of two or more of the above.

The term “prodrug” as used in this application refers to a precursor or derivative form of a pharmaceutically active substance that is less cytotoxic to tumor cells compared to the parent drug and is capable of being enzymatically activated or converted into the more active parent form. See, e.g., Wilman, “Prodrugs in Cancer Chemotherapy” Biochemical Society Transactions, 14, pp. 375-382, 615th Meeting Belfast (1986) and Stella et al., “Prodrugs: A Chemical Approach to Targeted Drug Delivery,” Directed Drug Delivery, Borchardt et al., (ed.), pp. 247-267, Humana Press (1985). The prodrugs of this invention include, but are not limited to, phosphate-containing prodrugs, thiophosphate-containing prodrugs, sulfate-containing prodrugs, peptide-containing prodrugs, D-amino acid-modified prodrugs, glycosylated prodrugs, β-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use in this invention include, but are not limited to, those chemotherapeutic agents described above.

A “growth inhibitory agent” when used herein refers to a compound or composition which inhibits growth of a cell (e.g., a cell whose growth is dependent upon FGFR3 expression either in vitro or in vivo). Examples of growth inhibitory agents include agents that block cell cycle progression (at a place other than S phase), such as agents that induce G1 arrest and M-phase arrest. Classical M-phase blockers include the vincas (vincristine and vinblastine), taxanes, and to FGFR3somerase II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogenes, and antineoplastic drugs” by Murakami et al. (WB Saunders: Philadelphia, 1995), especially p. 13. The taxanes (paclitaxel and docetaxel) are anticancer drugs both derived from the yew tree. Docetaxel (TAXOTERE®, Rhone-Poulenc Rorer), derived from the European yew, is a semisynthetic analogue of paclitaxel (TAXOL®, Bristol-Myers Squibb). Paclitaxel and docetaxel promote the assembly of microtubules from tubulin dimers and stabilize microtubules by preventing depolymerization, which results in the inhibition of mitosis in cells.

By “radiation therapy” is meant the use of directed gamma rays or beta rays to induce sufficient damage to a cell so as to limit its ability to function normally or to destroy the cell altogether. It will be appreciated that there will be many ways known in the art to determine the dosage and duration of treatment. Typical treatments are given as a one time administration and typical dosages range from 10 to 200 units (Grays) per day.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

The term “concurrently” is used herein to refer to administration of two or more therapeutic agents, where at least part of the administration overlaps in time. Accordingly, concurrent administration includes a dosing regimen when the administration of one or more agent(s) continues after discontinuing the administration of one or more other agent(s).

By “reduce or inhibit” is meant the ability to cause an overall decrease of 20%, 30%, 40%, 50%, 60%, 70%, 75%, 80%, 85%, 90%, 95%, or greater. Reduce or inhibit can refer to the symptoms of the disorder being treated, the presence or size of metastases, or the size of the primary tumor.

The term “package insert” is used to refer to instructions customarily included in commercial packages of therapeutic products, that contain information about the indications, usage, dosage, administration, combination therapy, contraindications and/or warnings concerning the use of such therapeutic products.

An “article of manufacture” is any manufacture (e.g., a package or container) or kit comprising at least one reagent, e.g., a medicament for treatment of a disease or disorder (e.g., cancer), or a probe for specifically detecting a biomarker described herein. In certain embodiments, the manufacture or kit is promoted, distributed, or sold as a unit for performing the methods described herein.

A “target audience” is a group of people or an institution to whom or to which a particular medicament is being promoted or intended to be promoted, as by marketing or advertising, especially for particular uses, treatments, or indications, such as individuals, populations, readers of newspapers, medical literature, and magazines, television or internet viewers, radio or internet listeners, physicians, drug companies, etc.

The phrase “based on” when used herein means that the information about one or more biomarkers is used to inform a treatment decision, information provided on a package insert, or marketing/promotional guidance, etc.

As is understood by one skilled in the art, reference to “about” a value or parameter herein includes (and describes) embodiments that are directed to that value or parameter per se. For example, description referring to “about X” includes description of “X”.

It is understood that aspect and embodiments described herein include “consisting” and/or “consisting essentially of” aspects and embodiments. As used herein, the singular form “a”, “an”, and “the” includes plural references unless indicated otherwise.

### II. Methods and Uses

Provided herein are methods utilizing a FGFR3 biomarker. In particular, methods utilizing a FGFR3 antagonist and a FGFR3 biomarker. For example provided are methods for treating an individual with disease or disorder comprising administering a therapeutically effective amount of a FGFR3 antagonist (e.g., anti-FGFR3 antibodies) to the individual if the individual has been found to have presence and/or elevated levels of a FGFR3 biomarker. Further provided herein are methods for treating a disease or disorder in an individual, the method comprising: determining that a sample from the individual comprises elevated levels of a FGFR3 biomarker, and administering an effective amount of a FGFR3 antagonist (e.g., anti-FGFR3 antibodies) to the individual, whereby the disease or disorder is treated. In some embodiments, the FGFR3 biomarker is FGFR3 expression. In some embodiments, the FGFR3 expression is FGFR3 polypeptide expression and FGFR3 polypeptide expression is determined in by IHC. In some embodiments, elevated levels of a FGFR3 biomarker is detected by IHC clinical diagnosis of positive or IHC clinical score of 1 or higher. In some embodiments, the disease or disorder is a proliferative disease or disorder. In some embodiments, the proliferative disease or disorder is cancer. In some embodiments, the cancer is a solid tumor. In some embodiments, the cancer is bladder cancer. In some embodiments, the cancer is transitional cell carcinoma (i.e., urothelial cell carcinoma).

Provided herein are methods of treating a disease or disorder in an individual comprising administering to the individual an effective amount of an FGFR3 antagonist (e.g., anti-FGFR3 antibodies), wherein treatment is based upon presence and/or elevated levels of a FGFR3 biomarker in a sample from the individual. In some embodiments, the FGFR3 biomarker is FGFR3 expression. In some embodiments, the FGFR3 expression is FGFR3 polypeptide expression and FGFR3 polypeptide expression is determined in by IHC. In some embodiments, elevated levels of a FGFR3 biomarker is detected by IHC clinical diagnosis of positive or IHC clinical score of 1 or higher. In some embodiments, the disease or disorder is a proliferative disease or disorder. In some embodiments, the proliferative disease or disorder is cancer. In some embodiments, the cancer is a solid tumor. In some embodiments, the cancer is bladder cancer. In some embodiments, the cancer is transitional cell carcinoma (i.e., urothelial cell carcinoma).

In addition, provided herein are methods for selecting a therapy for an individual with a disease or disorder comprising determining presence and/or levels of a FGFR3 biomarker, and selecting a medicament based on the presence and/or levels of the biomarker. In some embodiments, the medicament is selected based upon elevated levels of the FGFR3 biomarker. In some embodiments, the FGFR3 biomarker is FGFR3 expression. In some embodiments, the FGFR3 expression is FGFR3 polypeptide expression and FGFR3 polypeptide expression is determined in by IHC. In some embodiments, elevated levels of a FGFR3 biomarker is detected by IHC clinical diagnosis of positive or IHC clinical score of 1 or higher. In some embodiments, the disease or disorder is a proliferative disease or disorder. In some embodiments, the proliferative disease or disorder is cancer. In some embodiments, the cancer is a solid tumor. In some embodiments, the cancer is bladder cancer. In some embodiments, the cancer is transitional cell carcinoma (i.e., urothelial cell carcinoma).

Provided herein are methods of identifying an individual with a disease or disorder who is more or less likely to exhibit benefit from treatment comprising a FGFR3 antagonist (e.g., anti-FGFR3 antibodies), the method comprising: determining presence and/or levels of a FGFR3 biomarker in a sample from the individual, wherein the presence and/or elevated levels of the FGFR3 biomarker in the sample indicates that the individual is more likely to exhibit benefit from treatment comprising the FGFR3 antagonist (e.g., anti-FGFR3 antibodies) or absence and/or reduced levels of the FGFR3 biomarker indicates that the individual is less likely to exhibit benefit from treatment comprising the FGFR3 antagonist (e.g., anti-FGFR3 antibodies). In some embodiments, the FGFR3 biomarker is FGFR3 expression. In some embodiments, the FGFR3 expression is FGFR3 polypeptide expression and FGFR3 polypeptide expression is determined in by IHC. In some embodiments, elevated levels of a FGFR3 biomarker is detected by IHC clinical diagnosis of positive or IHC clinical score of 1 or higher. In some embodiments, the disease or disorder is a proliferative disease or disorder. In some embodiments, the proliferative disease or disorder is cancer. In some embodiments, the cancer is a solid tumor. In some embodiments, the cancer is bladder cancer. In some embodiments, the cancer is transitional cell carcinoma (i.e., urothelial cell carcinoma).

Further provided herein are methods for advertising a FGFR3 antagonist (e.g., anti-FGFR3 antibodies) comprising promoting, to a target audience, the use of the FGFR3 antagonist (e.g., anti-FGFR3 antibodies) for treating an individual with a disease or disorder based on presence and/or levels of a FGFR3 biomarker. In some embodiments, the use of the FGFR3 antagonist is based upon elevated levels of the FGFR3 biomarker. In some embodiments, the FGFR3 biomarker is FGFR3 expression. In some embodiments, the FGFR3 expression is FGFR3 polypeptide expression and FGFR3 polypeptide expression is determined in by IHC. In some embodiments, elevated levels of a FGFR3 biomarker is detected by IHC clinical diagnosis of positive or IHC clinical score of 1 or higher. In some embodiments, the disease or disorder is a proliferative disease or disorder. In some embodiments, the proliferative disease or disorder is cancer. In some embodiments, the cancer is a solid tumor. In some embodiments, the cancer is bladder cancer. In some embodiments, the cancer is transitional cell carcinoma (i.e., urothelial cell carcinoma).

Provided herein are also assays for identifying an individual with a disease or disorder to receive a FGFR3 antagonist (e.g., anti-FGFR3 antibodies), the method comprising: (a) determining presence and/or levels of a FGFR3 biomarker in a sample from the individual; (b) recommending a FGFR3 antagonist (e.g., anti-FGFR3 antibodies) based upon the presence and/or levels of the FGFR3 biomarker. In some embodiments, the FGFR3 antagonist is recommended based upon elevated levels of the FGFR3 biomarker. In some embodiments, the FGFR3 biomarker is FGFR3 expression. In some embodiments, the FGFR3 expression is FGFR3 polypeptide expression and FGFR3 polypeptide expression is determined in by IHC. In some embodiments, elevated levels of a FGFR3 biomarker is detected by IHC clinical diagnosis of positive or IHC clinical score of 1 or higher. In some embodiments, the disease or disorder is a proliferative disease or disorder. In some embodiments, the proliferative disease or disorder is cancer. In some embodiments, the cancer is a solid tumor. In some embodiments, the cancer is bladder cancer. In some embodiments, the cancer is transitional cell carcinoma (i.e., urothelial cell carcinoma).

Provided herein are diagnostic kits comprising one or more reagent for determining levels of a FGFR3 biomarker in a sample from an individual with a disease or disorder, wherein detection of presence and/or elevated levels of the FGFR3 biomarker means increased efficacy when the individual is treated with a FGFR3 antagonist (e.g., anti-FGFR3 antibodies), and wherein detection of a low or substantially undetectable levels of a FGFR3 biomarker means a decreased efficacy when the individual with the disease is treated with the FGFR3 antagonist (e.g., anti-FGFR3 antibodies). Provided herein are also articles of manufacture comprising, packaged together, a FGFR3 antagonist (e.g., anti-FGFR3 antibodies) in a pharmaceutically acceptable carrier and a package insert indicating that the FGFR3 antagonist (e.g., anti-FGFR3 antibodies) is for treating a patient with a disease or disorder based on expression of a FGFR3 biomarker. Treatment methods include any of the treatment methods disclosed herein. Further provided are the invention concerns a method for manufacturing an article of manufacture comprising combining in a package a pharmaceutical composition comprising a FGFR3 antagonist (e.g., anti-FGFR3 antibodies) and a package insert indicating that the pharmaceutical composition is for treating a patient with a disease or disorder based on expression of FGFR3 biomarker. In some embodiments, the FGFR3 biomarker is FGFR3 expression. In some embodiments, the FGFR3 expression is FGFR3 polypeptide expression and FGFR3 polypeptide expression is determined in by IHC. In some embodiments, elevated levels of a FGFR3 biomarker is detected by IHC clinical diagnosis of positive or IHC clinical score of 1 or higher. In some embodiments, the disease or disorder is a proliferative disease or disorder. In some embodiments, the proliferative disease or disorder is cancer. In some embodiments, the cancer is a solid tumor. In some embodiments, the cancer is bladder cancer. In some embodiments, the cancer is transitional cell carcinoma (i.e., urothelial cell carcinoma).

Further provided herein are methods for treating a disease or disorder in an individual comprising administering to the individual an effective amount of a FGFR3 antagonist (e.g., anti-FGFR3 antibodies) and assessing levels of one or more FGFR3 biomarkers in a sample from the individual (e.g., compared to a reference) during treatment with the FGFR3 antagonist (e.g., anti-FGFR3 antibodies). Also provided are methods of treating a disease or disorder in an individual comprising administering to the individual an effective amount of a FGFR3 antagonist (e.g., anti-FGFR3 antibodies), wherein treatment is based upon levels of one or more FGFR3 biomarkers in a sample from the individual (e.g., compared to a reference). Provided are methods of monitor responsiveness in an individual to treatment comprising a FGFR3 antagonist (e.g., anti-FGFR3 antibodies), the method comprising: determining levels of one or more FGFR3 biomarkers in a sample from the individual, wherein reduced levels of one or more FGFR3 biomarkers (e.g., compared to a reference) in the sample indicates that the individual is more likely responsive to treatment comprising the FGFR3 antagonist (e.g., anti-FGFR3 antibodies) or elevated levels and/or levels substantially the same as pretreatment levels of one or more FGFR3 biomarkers (e.g., compared to a reference) indicates that the individual is less likely responsive to treatment comprising the FGFR3 antagonist (e.g., anti-FGFR3 antibodies). Additionally provided are methods of determining whether an individual with a disease or disorder should continue or discontinue treatment comprising a FGFR3 antagonist (e.g., anti-FGFR3 antibodies), the method comprising measuring in a sample from the individual levels of one or more FGFR3 biomarkers, wherein elevated levels and/or levels substantially the same as pretreatment levels of one or more FGFR3 biomarkers (e.g., compared to a reference) determines the individual should discontinue treatment comprising the FGFR3 antagonist (e.g., anti-FGFR3 antibodies) and reduced levels of one or more FGFR3 biomarkers (e.g., compared to a reference) determines the individual should continue treatment comprising the FGFR3 antagonist (e.g., anti-FGFR3 antibodies).

In some embodiments of any of the methods, the one or more FGFR3 biomarker is one or more biomarkers selected from the group consisting of FABP4, PLAT, DUSP6, FGFBP1, SCNN1B, TRIM22, UPK1A, ID2, LDLR, LOXL1, IDI1, SEPP1, FDFT1, CCDC85A, MUC15, SC4MOL, CRISP3, S100A2, ERP27, FRAS1, PCSK9, SQLE, CYP4B1, IGHA1, MMP1, F2R, TSPAN12, ABP1, COL4A4, INSIG1, SLCO4A1, PDE8B, ATP1A4, CLDN8, NT5E, TNS1, VSIG2, PHLDA1, SCNN1G, COL4A2, FGFR3, HMGCS1, S100A9, VTCN1, CCDC80, SPATA17, MAN1A1, SPOCK1, SULF2, ACAT2, MUC20, MMP10, TMC4, HMGCR, CDK14, FASN, ATP6V1B1, DHRS2, TNS3, ATP2B4, PDZK1, MYCL1, CYB5B, KRT15, DAPL1, FAR2, DHCR7, ASPH, CFD, IFIT1, MR1, OLR1, C3orf58, DHRS9, IQGAP2, PPP1R3B, HS3ST1, C16orf54, FGD3, PIK31P1, LGALS8, OPTN, LAMB3, SCD, GKN1, MICB, ID1, SPTLC3, ETV4, ACSL3, SLC20A1, TSC22D3, DBP, IGFBP5, CYP1B1, CDC42EP3, SLC35A1, ID3, ITGA2, FOXO6, NDRG1, TBX3, SEZ6L2, WNT4, HOXA5, LRP8, PAICS, C10orf54, ELOVL5, CTNNAL1, SEMA3E, PFKFB3, KITLG, BCL11A, NEBL, TIMP2, STARD5, IL1RN, PCDHB14, MVP, TMEM47, CHAC2, OLFML2A, GDA, MMD, ALDH3B1, NME1, CLU, APOBEC3G, DDX39A, HBEGF, PNP, FDPS, FAM171B, ERO1L, ADORA2B, CYP51A1, TUBG1, LSS, STOX2, CTPS, ABAT, SEPW1, GABRP, TACC3, TCF7L1, TFPI2, FYB, MATN2, WNT100A, TFRC, RIMS2, PSMD14, GRHL3, ZFP36L1, TSGA10, GART, SLC45A3, ATL1, ANKDD1A, ACPL2, ITLN1, C20orf114, ARHGAP26, CYP24A1, HIST1H2AC, FAM49A, PLD1, TMPRSS2, PP14571, MAFB, SDR16C5, WDR4, TNIK, FAM46A, FAM134B, SEMA5A, PRICKLE1, ID4, PPP2R2B, MGC16075, ZNF404, IFI44, SMPDL3A, JDP2, CD55, ZIC2, C6orf141, CPAMD8, ME1, GGT6, C17orf103, FAM84A, CLIC5, KAL1, APCDD1, MT1F, MPPED2, SYNPO, TRIM16, TSPAN8, ARNT, DAPK2, SH3BGRL, PLK1, MBIP, METRNL, ANXA3, GSN, LIPG, PPIL1, SYTL5, UPK3B, SYNE1, PLSCR4, PTGER4, GMFG, MAFF, TMEM37, HCFC1R1, ZDHHC8P1, AXL, HLA-E, MVK, CASQ1, EBP, DNAJC4, BTN3A3, LRMP, IRF9, ART3, LYAR, SNRPD1, UPK2, MTHFD1L, EGFL6, BST2, LOC283788, AGPAT5, SERPINF1, CTSS, PROS1, TFF1, GJB2, TBC1D9, C9orf40, IPO5, LOC100289610, GPC3, PDK4, NFKBIA, CASZ1, SNCG, TIPIN, EPHA4, BAMBI, LMO4, PIK3C3, CXCL11, IL1R1, HSD17B2, PEA15, IRAK2, PRODH, CYP26B1, WDR78, WLS, SGSH, KLF9, CHORDC1, TRPC1, HS6ST3, ETV5, TRIM31, COL4A1, C3orf26, RPS6KA6, BMP2, SSFA2, TMCC3, IL1RAP, BBOX1, TMEM27, PDSS1, DSE, NR3C1, CPEB2, TPRG1, C15orf57, MGAM, HAMP, TLR4, GABRB3, GATA6, CLCN4, ZNF763, ACP1, GIMAP2, LOC284837, SNRPN, MBD5, CD109, JSRP1, TMEM151B, PIWIL1, FAM65B, EML5, COL4A3, PRKD2, MATR3, ACER3, NCRNA00247, and LOC100507557. In some embodiments, the FGFR3 biomaker is MMP1. In some embodiments, the FGFR3 biomarker is MMP10. In some embodiments, the sample is a urine sample. In some embodiments, the sample is a blood sample. In some embodiments, the disease or disorder is a proliferative disease or disorder. In some embodiments, the proliferative disease or disorder is cancer. In some embodiments, the cancer is a solid tumor. In some embodiments, the cancer is bladder cancer. In some embodiments, the cancer is transitional cell carcinoma (i.e., urothelial cell carcinoma).

Presence and/or expression levels/amount of a biomarker (e.g., FGFR3) can be determined qualitatively and/or quantitatively based on any suitable criterion known in the art, including but not limited to DNA, mRNA, cDNA, proteins, protein fragments and/or gene copy number. In certain embodiments, presence and/or expression levels/amount of a biomarker in a first sample is increased as compared to presence/absence and/or expression levels/amount in a second sample. In certain embodiments, presence/absence and/or expression levels/amount of a biomarker in a first sample is decreased as compared to presence and/or expression levels/amount in a second sample. In certain embodiments, the second sample is a reference sample, reference cell, reference tissue, control sample, control cell, or control tissue. Additional disclosures for determining presence/absence and/or expression levels/amount of a gene are described herein.

In some embodiments of any of the methods, elevated expression refers to an overall increase of about any of 10%, 20%, 30%, 40%, 50%, 60%, 70%, 80%, 90%, 95%, 96%, 97%, 98%, 99% or greater, in the level of biomarker (e.g., protein or nucleic acid (e.g., gene or mRNA)), detected by standard art known methods such as those described herein, as compared to a reference sample, reference cell, reference tissue, control sample, control cell, or control tissue. In certain embodiments, the elevated expression refers to the increase in expression level/amount of a biomarker in the sample wherein the increase is at least about any of 1.5×, 1.75×, 2×, 3×, 4×, 5×, 6×, 7×, 8×, 9×, 10×, 25×, 50×, 75×, or 100× the expression level/amount of the respective biomarker in a reference sample, reference cell, reference tissue, control sample, control cell, or control tissue. In some embodiments, elevated expression refers to an overall increase of greater than about 1.5 fold, about 1.75 fold, about 2 fold, about 2.25 fold, about 2.5 fold, about 2.75 fold, about 3.0 fold, or about 3.25 fold as compared to a reference sample, reference cell, reference tissue, control sample, control cell, control tissue, or internal control (e.g., housekeeping gene).

In some embodiments of any of the methods, reduced expression refers to an overall reduction of about any of 10%, 20%, 30%, 40%, 50%, 60%, 70%, 80%, 90%, 95%, 96%, 97%, 98%, 99% or greater, in the level of biomarker (e.g., protein or nucleic acid (e.g., gene or mRNA)), detected by standard art known methods such as those described herein, as compared to a reference sample, reference cell, reference tissue, control sample, control cell, or control tissue. In certain embodiments, reduced expression refers to the decrease in expression level/amount of a biomarker in the sample wherein the decrease is at least about any of 0.9×, 0.8×, 0.7×, 0.6×, 0.5×, 0.4×, 0.3×, 0.2×, 0.1×, 0.05×, or 0.01× the expression level/amount of the respective biomarker in a reference sample, reference cell, reference tissue, control sample, control cell, or control tissue.

Presence and/or expression level/amount of various biomarkers in a sample can be analyzed by a number of methodologies, many of which are known in the art and understood by the skilled artisan, including, but not limited to, immunohistochemistry (“IHC”), Western blot analysis, immunoprecipitation, molecular binding assays, ELISA, ELIFA, fluorescence activated cell sorting (“FACS”), MassARRAY, proteomics, quantitative blood based assays (as for example Serum ELISA), biochemical enzymatic activity assays, in situ hybridization, Southern analysis, Northern analysis, whole genome sequencing, polymerase chain reaction (“PCR”) including quantitative real time PCR (“qRT-PCR”) and other amplification type detection methods, such as, for example, branched DNA, SISBA, TMA and the like), RNA-Seq, FISH, microarray analysis, gene expression profiling, and/or serial analysis of gene expression (“SAGE”), as well as any one of the wide variety of assays that can be performed by protein, gene, and/or tissue array analysis. Typical protocols for evaluating the status of genes and gene products are found, for example in Ausubel et al., eds., 1995, Current Protocols In Molecular Biology, Units 2 (Northern Blotting), 4 (Southern Blotting), 15 (Immunoblotting) and 18 (PCR Analysis). Multiplexed immunoassays such as those available from Rules Based Medicine or Meso Scale Discovery (“MSD”) may also be used.

In some embodiments, presence and/or expression level/amount of a biomarker is determined using a method comprising: (a) performing gene expression profiling, PCR (such as rtPCR), RNA-seq, microarray analysis, SAGE, MassARRAY technique, or FISH on a sample (such as a subject cancer sample); and b) determining presence and/or expression level/amount of a biomarker in the sample. In some embodiments, the microarray method comprises the use of a microarray chip having one or more nucleic acid molecules that can hybridize under stringent conditions to a nucleic acid molecule encoding a gene mentioned above or having one or more polypeptides (such as peptides or antibodies) that can bind to one or more of the proteins encoded by the genes mentioned above. In one embodiment, the PCR method is qRT-PCR. In one embodiment, the PCR method is multiplex-PCR. In some embodiments, gene expression is measured by microarray. In some embodiments, gene expression is measured by qRT-PCR. In some embodiments, expression is measured by multiplex-PCR.

In some embodiments of any of the methods, assays and/or kits, the FGFR3 biomarker is FGFR3 mutation. In some embodiments, the FGFR3 mutation is encodes for one or more of the following FGFR3 amino acid variants: FGFR3R248C, FGFR3S249C, FGFR3G370C, FGFR3S371C, FGFR3Y373C, FGFR3G380R, FGFR3K650X (e.g., FGFR3K650E), FGFR3K650M, and FGFR3G697C. In some embodiments, the FGFR3 mutation is one or more of the following FGFR3 amino acid variants: FGFR3 c.746C>G, FGFR3 c.1118A>G, FGFR3 c.742C>T, FGFR3 c.1108G>T, FGFR3 c.1111A>T.

Methods for the evaluation of mRNAs in cells are well known and include, for example, hybridization assays using complementary DNA probes (such as in situ hybridization using labeled riboprobes specific for the one or more genes, Northern blot and related techniques) and various nucleic acid amplification assays (such as RT-PCR using complementary primers specific for one or more of the genes, and other amplification type detection methods, such as, for example, branched DNA, SISBA, TMA and the like).

Samples from mammals can be conveniently assayed for mRNAs using Northern, dot blot or PCR analysis. In addition, such methods can include one or more steps that allow one to determine the levels of target mRNA in a biological sample (e.g., by simultaneously examining the levels a comparative control mRNA sequence of a “housekeeping” gene such as an actin family member). Optionally, the sequence of the amplified target cDNA can be determined.

Optional methods include protocols which examine or detect mRNAs, such as target mRNAs, in a tissue or cell sample by microarray technologies. Using nucleic acid microarrays, test and control mRNA samples from test and control tissue samples are reverse transcribed and labeled to generate cDNA probes. The probes are then hybridized to an array of nucleic acids immobilized on a solid support. The array is configured such that the sequence and position of each member of the array is known. For example, a selection of genes whose expression correlates with increased or reduced clinical benefit of anti-angiogenic therapy may be arrayed on a solid support. Hybridization of a labeled probe with a particular array member indicates that the sample from which the probe was derived expresses that gene.

According to some embodiments, presence and/or expression level/amount is measured by observing protein expression levels of an aforementioned gene. In certain embodiments, the method comprises contacting the biological sample with antibodies to a biomarker (e.g., anti-FGFR3 antibodies) described herein under conditions permissive for binding of the biomarker, and detecting whether a complex is formed between the antibodies and biomarker. Such method may be an in vitro or in vivo method. In one embodiment, an antibody is used to select subjects eligible for therapy with FGFR3 antagonist e.g., a biomarker for selection of individuals.

In certain embodiments, the presence and/or expression level/amount of biomarker proteins in a sample is examined using IHC and staining protocols. IHC staining of tissue sections has been shown to be a reliable method of determining or detecting presence of proteins in a sample. In some embodiments of any of the methods, assays and/or kits, the FGFR3 biomarker is FGFR3. In some embodiments, FGFR3 is detected by immunohistochemistry. In some embodiments, elevated expression of a FGFR3 biomarker in a sample from an individual is elevated protein expression and, in further embodiments, is determined using IHC. In one embodiment, expression level of biomarker is determined using a method comprising: (a) performing IHC analysis of a sample (such as a subject cancer sample) with an antibody; and b) determining expression level of a biomarker in the sample. In some embodiments, IHC staining intensity is determined relative to a reference. In some embodiments, the reference is a reference value. In some embodiments, the reference is a reference sample (e.g., control cell line staining sample)

IHC may be performed in combination with additional techniques such as morphological staining and/or fluorescence in-situ hybridization. Two general methods of IHC are available; direct and indirect assays. According to the first assay, binding of antibody to the target antigen is determined directly. This direct assay uses a labeled reagent, such as a fluorescent tag or an enzyme-labeled primary antibody, which can be visualized without further antibody interaction. In a typical indirect assay, unconjugated primary antibody binds to the antigen and then a labeled secondary antibody binds to the primary antibody. Where the secondary antibody is conjugated to an enzymatic label, a chromogenic or fluorogenic substrate is added to provide visualization of the antigen. Signal amplification occurs because several secondary antibodies may react with different epitopes on the primary antibody.

The primary and/or secondary antibody used for IHC typically will be labeled with a detectable moiety. Numerous labels are available which can be generally grouped into the following categories: (a) Radioisotopes, such as ³⁵S, ¹⁴C, ¹²⁵I, ³H, and ¹³¹I; (b) colloidal gold particles; (c) fluorescent labels including, but are not limited to, rare earth chelates (europium chelates), Texas Red, rhodamine, fluorescein, dansyl, Lissamine, umbelliferone, phycocrytherin, phycocyanin, or commercially available fluorophores such SPECTRUM ORANGE7 and SPECTRUM GREEN7 and/or derivatives of any one or more of the above; (d) various enzyme-substrate labels are available and U.S. Pat. No. 4,275,149 provides a review of some of these. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, β-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like.

Examples of enzyme-substrate combinations include, for example, horseradish peroxidase (HRPO) with hydrogen peroxidase as a substrate; alkaline phosphatase (AP) with para-Nitrophenyl phosphate as chromogenic substrate; and β-D-galactosidase (β-D-Gal) with a chromogenic substrate (e.g., p-nitrophenyl-β-D-galactosidase) or fluorogenic substrate (e.g., 4-methylumbelliferyl-β-D-galactosidase). For a general review of these, see U.S. Pat. Nos. 4,275,149 and 4,318,980.

In some embodiments of any of the methods, FGFR3 is detected by immunohistochemistry using an anti-FGFR3 diagnostic antibody (i.e., primary antibody). In some embodiments, the FGFR3 diagnostic antibody specifically binds human FGFR3. In some embodiments, the FGFR3 diagnostic antibody specifically binds an epitope comprising amino acids 25-124 of human FGFR3. In some embodiments, the FGFR3 diagnostic antibody specifically binds an epitope comprising LGTEQRVVGRAAEV PGPEPGQQEQLVFGSGDAVELSCPPPGGGPMGPTVWVKDGTGLVPSERVLVGPQRLQVLNASHE DSGAYSCRQRLTQRVLCHFSVR (SEQ ID NO: 181). In some embodiments of any of the FGFR3 diagnostic antibodies, the FGFR3 diagnostic antibody is a nonhuman antibody. In some embodiments of any of the FGFR3 diagnostic antibodies, the FGFR3 diagnostic antibody is a rat, mouse, or rabbit antibody. In some embodiments of any of the FGFR3 diagnostic antibodies, the FGFR3 diagnostic antibody is a monoclonal antibody. In some embodiments of any of the FGFR3 diagnostic antibodies, the FGFR3 diagnostic antibody is an IgG2 antibody. In some embodiments of any of the FGFR3 diagnostic antibodies, the FGFR3 diagnostic antibody is an IgG2a antibody. In some embodiments of any of the FGFR3 diagnostic antibodies, the FGFR3 diagnostic antibody is sc-13121 (i.e., B-9) from Santa Cruz Biotechnology. In some embodiments, the FGFR3 diagnostic antibody is directly labeled.

Specimens thus prepared may be mounted and coverslipped. Slide evaluation is then determined, e.g., using a microscope, and staining intensity criteria, routinely used in the art, may be employed. In some embodiments, a staining pattern score of about 1+ or higher is diagnostic and/or prognostic. In certain embodiments, a staining pattern score of about 2+ or higher in an IHC assay is diagnostic and/or prognostic. In other embodiments, a staining pattern score of about 3 or higher is diagnostic and/or prognostic. In one embodiment, it is understood that when cells and/or tissue from a tumor or colon adenoma are examined using IHC, staining is generally determined or assessed in tumor cell and/or tissue (as opposed to stromal or surrounding tissue that may be present in the sample). In some embodiments, elevated levels of a FGFR3 biomarker is detected by IHC clinical diagnosis of positive or IHC clinical score of 1 or higher. In some embodiments, the IHC clinical score of 1 or higher is 2 or higher. In some embodiments, the IHC clinical score of 1 or higher is 3. In some embodiments, the IHC clinical score is 3. In some embodiments, the IHC clinical score is 2 or 3.

In some embodiments of any of the methods, assays, and/or kits, an IHC clinical score of 1 represents a) >10% cytoplasmic and/or membrane staining and b) weak cytoplasmic and/or membrane staining with moderate and/or strong staining being <10% of positively stained cells. In some embodiments, an IHC clinical score of 1 represents staining similar to and/or substantially the same as RPMI8226 cell line staining. In some embodiments, an IHC clinical score of 2 represents a) >10% cytoplasmic and/or membrane staining and b) moderate cytoplasmic and/or membrane staining in >10% of cells, with strong staining being <10% of positively stained cells; weak staining may or may not be present. In some embodiments, an IHC clinical score of 2 represents staining similar to and/or substantially the same as OPM2 cell line staining. In some embodiments, an IHC clinical score of 3 represents a) >10% cytoplasmic and/or membrane staining and b) strong cytoplasmic and/or membrane staining in >10% of positively staining cells; weak and moderate staining may or may not be present. In some embodiments, an IHC clinical score of 3 represents staining similar to and/or substantially the same as KMS11 cell line staining. In some embodiments of any of the IHC methods, the IHC clinical score is determined using an FGFR3 diagnostic antibody as described herein.

In alternative methods, the sample may be contacted with an antibody specific for said biomarker under conditions sufficient for an antibody-biomarker complex to form, and then detecting said complex. The presence of the biomarker may be detected in a number of ways, such as by Western blotting and ELISA procedures for assaying a wide variety of tissues and samples, including plasma or serum. A wide range of immunoassay techniques using such an assay format are available, see, e.g., U.S. Pat. Nos. 4,016,043, 4,424,279 and 4,018,653. These include both single-site and two-site or “sandwich” assays of the non-competitive types, as well as in the traditional competitive binding assays. These assays also include direct binding of a labeled antibody to a target biomarker.

Presence and/or expression level/amount of a selected biomarker in a tissue or cell sample may also be examined by way of functional or activity-based assays. For instance, if the biomarker is an enzyme, one may conduct assays known in the art to determine or detect the presence of the given enzymatic activity in the tissue or cell sample.

In certain embodiments, the samples are normalized for both differences in the amount of the biomarker assayed and variability in the quality of the samples used, and variability between assay runs. Such normalization may be accomplished by detecting and incorporating the expression of certain normalizing biomarkers, including well known housekeeping genes, such as ACTB. Alternatively, normalization can be based on the mean or median signal of all of the assayed genes or a large subset thereof (global normalization approach). On a gene-by-gene basis, measured normalized amount of a subject tumor mRNA or protein is compared to the amount found in a reference set. Normalized expression levels for each mRNA or protein per tested tumor per subject can be expressed as a percentage of the expression level measured in the reference set. The presence and/or expression level/amount measured in a particular subject sample to be analyzed will fall at some percentile within this range, which can be determined by methods well known in the art.

In certain embodiments, relative expression level of a gene is determined as follows:

Relative expression gene1 sample1=2 exp (Ct housekeeping gene−Ct gene1) with Ct determined in a sample.
Relative expression gene1 reference RNA=2 exp (Ct housekeeping gene−Ct gene1) with Ct determined in the reference sample.
Normalized relative expression gene1 sample1=(relative expression gene1 sample1/relative expression gene1 reference RNA)×100
Ct is the threshold cycle. The Ct is the cycle number at which the fluorescence generated within a reaction crosses the threshold line.

All experiments are normalized to a reference RNA, which is a comprehensive mix of RNA from various tissue sources (e.g., reference RNA #636538 from Clontech, Mountain View, Calif.). Identical reference RNA is included in each qRT-PCR run, allowing comparison of results between different experimental runs.

In one embodiment, the sample is a clinical sample. In another embodiment, the sample is used in a diagnostic assay. In some embodiments, the sample is obtained from a primary or metastatic tumor. Tissue biopsy is often used to obtain a representative piece of tumor tissue. Alternatively, tumor cells can be obtained indirectly in the form of tissues or fluids that are known or thought to contain the tumor cells of interest. For instance, samples of lung cancer lesions may be obtained by resection, bronchoscopy, fine needle aspiration, bronchial brushings, or from sputum, pleural fluid or blood. Genes or gene products can be detected from cancer or tumor tissue or from other body samples such as urine, sputum, serum or plasma. The same techniques discussed above for detection of target genes or gene products in cancerous samples can be applied to other body samples. Cancer cells may be sloughed off from cancer lesions and appear in such body samples. By screening such body samples, a simple early diagnosis can be achieved for these cancers. In addition, the progress of therapy can be monitored more easily by testing such body samples for target genes or gene products.

In certain embodiments, a reference sample, reference cell, reference tissue, control sample, control cell, or control tissue is a single sample or combined multiple samples from the same subject or individual that are obtained at one or more different time points than when the test sample is obtained. For example, a reference sample, reference cell, reference tissue, control sample, control cell, or control tissue is obtained at an earlier time point from the same subject or individual than when the test sample is obtained. Such reference sample, reference cell, reference tissue, control sample, control cell, or control tissue may be useful if the reference sample is obtained during initial diagnosis of cancer and the test sample is later obtained when the cancer becomes metastatic.

In certain embodiments, a reference sample, reference cell, reference tissue, control sample, control cell, or control tissue is a combined multiple samples from one or more healthy individuals who are not the subject or individual. In certain embodiments, a reference sample, reference cell, reference tissue, control sample, control cell, or control tissue is a combined multiple samples from one or more individuals with a disease or disorder (e.g., cancer) who are not the subject or individual. In certain embodiments, a reference sample, reference cell, reference tissue, control sample, control cell, or control tissue is pooled RNA samples from normal tissues or pooled plasma or serum samples from one or more individuals who are not the subject or individual. In certain embodiments, a reference sample, reference cell, reference tissue, control sample, control cell, or control tissue is pooled RNA samples from tumor tissues or pooled plasma or serum samples from one or more individuals with a disease or disorder (e.g., cancer) who are not the subject or individual.

In certain embodiments, a reference sample, reference cell, reference tissue, control sample, control cell, or control tissue is a sample cell line. In certain embodiments, a reference sample, reference cell, reference tissue, control sample, control cell, or control tissue is RPMI8226, KSM11, and/or OPM2.

In some embodiments, the sample is a tissue sample from the individual. In some embodiments, the tissue sample is a tumor tissue sample (e.g., biopsy tissue). In some embodiments, the tissue sample is bladder tissue. In some embodiments, the tissue sample is urothelial tissue. In some embodiments, the tissue sample is tissue adjacent the bladder.

In some embodiments of any of the methods, the disease or disorder is a tumor. In some embodiments, the tumor is a malignant cancerous tumor (i.e., cancer). In some embodiments, the tumor and/or cancer is a solid tumor or a non-solid or soft tissue tumor. Examples of soft tissue tumors include leukemia (e.g., chronic myelogenous leukemia, acute myelogenous leukemia, adult acute lymphoblastic leukemia, acute myelogenous leukemia, mature B-cell acute lymphoblastic leukemia, chronic lymphocytic leukemia, polymphocytic leukemia, or hairy cell leukemia) or lymphoma (e.g., non-Hodgkin's lymphoma, cutaneous T-cell lymphoma, or Hodgkin's disease). A solid tumor includes any cancer of body tissues other than blood, bone marrow, or the lymphatic system. Solid tumors can be further divided into those of epithelial cell origin and those of non-epithelial cell origin. Examples of epithelial cell solid tumors include tumors of the gastrointestinal tract, colon, colorectal (e.g., basaloid colorectal carcinoma), breast, prostate, lung, kidney, liver, pancreas, ovary (e.g., endometrioid ovarian carcinoma), head and neck, oral cavity, stomach, duodenum, small intestine, large intestine, anus, gall bladder, labium, nasopharynx, skin, uterus, male genital organ, urinary organs (e.g., urothelium carcinoma, dysplastic urothelium carcinoma, transitional cell carcinoma), bladder, and skin. Solid tumors of non-epithelial origin include sarcomas, brain tumors, and bone tumors. In some embodiments, the cancer is transitional cell carcinoma or urothelium carcinoma. In some embodiments, the cancer is squamous cell carcinoma. In some embodiments, the cancer is adenocarcinoma. Other examples of tumors are described in the Definitions section.

In some embodiments of any of the methods, the FGFR3 antagonist is an antibody, binding polypeptide, binding small molecule, or polynucleotide. In some embodiments, the FGFR3 antagonist is an antibody. In some embodiments, the antibody is a monoclonal antibody. In some embodiments, the antibody is a human, humanized, or chimeric antibody. In some embodiments, the antibody is an antibody fragment and the antibody fragment binds FGFR3.

In some embodiments of any of the methods, the individual according to any of the above embodiments may be a human.

In a further embodiment, provided herein are methods for treating a cancer. In one embodiment, the method comprises administering to an individual having such cancer an effective amount of a FGFR3 antagonist. In one such embodiment, the method further comprises administering to the individual an effective amount of at least one additional therapeutic agent, as described below. In some embodiments, the individual may be a human.

FGFR3 antagonists described herein can be used either alone or in combination with other agents in a therapy. For instance, a FGFR3 antagonist described herein may be co-administered with at least one additional therapeutic agent. In certain embodiments, an additional therapeutic agent is a chemotherapeutic agent.

Such combination therapies noted above encompass combined administration (where two or more therapeutic agents are included in the same or separate formulations), and separate administration, in which case, administration of the antagonist can occur prior to, simultaneously, and/or following, administration of the additional therapeutic agent and/or adjuvant. FGFR3 antagonists described herein can also be used in combination with radiation therapy.

A FGFR3 antagonist (e.g., an antibody, binding polypeptide, and/or small molecule) described herein (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

FGFR3 antagonists (e.g., an antibody, binding polypeptide, and/or small molecule) described herein may be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The FGFR3 antagonist need not be, but is optionally formulated with one or more agents currently used to prevent or treat the disorder in question. The effective amount of such other agents depends on the amount of the FGFR3 antagonist present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of a FGFR3 antagonist described herein (when used alone or in combination with one or more other additional therapeutic agents) will depend on the type of disease to be treated, the severity and course of the disease, whether the FGFR3 antagonist is administered for preventive or therapeutic purposes, previous therapy, the patient's clinical history and response to the FGFR3 antagonist, and the discretion of the attending physician. The FGFR3 antagonist is suitably administered to the patient at one time or over a series of treatments. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. Such doses may be administered intermittently, e.g., every week or every three weeks (e.g., such that the patient receives from about two to about twenty, or e.g., about six doses of the FGFR3 antagonist). An initial higher loading dose, followed by one or more lower doses may be administered. An exemplary dosing regimen comprises administering. However, other dosage regimens may be useful. The progress of this therapy is easily monitored by conventional techniques and assays.

In some embodiments of any of the methods, the FGFR3 antagonist (e.g., anti-FGFR3 antibody) is administered at a dosage of about 2-30 mg/kg. In some embodiments, the FGFR3 antagonist (e.g., anti-FGFR3 antibody) is administered at a dosage of about any of 2 mg/kg, 4 mg/kg, 8 mg/kg, 15 mg/kg, or 30 mg/kg. In some embodiments, the FGFR3 antagonist (e.g., anti-FGFR3 antibody) is administered at a dosage of about any of 2 mg/kg, 4 mg/kg, 8 mg/kg, 15 mg/kg, or 30 mg/kg in 28-day cycles. In some embodiments, the FGFR3 antagonist (e.g., anti-FGFR3 antibody) is administered with a loading dose on cycle 1, day 8 at a dosage of about any of 2 mg/kg, 4 mg/kg, 8 mg/kg, 15 mg/kg, or 30 mg/kg.

It is understood that any of the above formulations or therapeutic methods may be carried out using an immunoconjugate in place of or in addition to the FGFR3 antagonist.

### III. Therapeutic Compositions

In another embodiment, provided herein are FGFR3 antagonists useful in the methods described herein. In some embodiments, the FGFR3 antagonists are an antibody, binding polypeptide, binding small molecule, and/or polynucleotide.

In some embodiments, provided herein are FGFR3 antagonists that bind to FGFR3. In some embodiments, the FGFR3 antagonist binds a FGFR3 IIIb isoform and/or a FGFR3 IIIc isoform. In some embodiments, the FGFR3 antagonist binds a mutated FGFR3 (e.g., one or more of FGFR3 IIIb R248C, S249C, G372C, Y375C, K652E, and/or one or more of FGFR3 IIIc R248C, S249C, G370C, Y373C, K650E). In some embodiments, the FGFR3 antagonist binds monomeric FGFR3 (e.g., monomeric FGFR3 IIIb and/or IIIc isoforms). In some embodiments, the FGFR3 antagonist promotes formation of monomeric FGFR3, such as by stabilizing the monomeric FGFR3 form relative to the dimeric FGFR3 form.

In some embodiments, the FGFR3 antagonist inhibits constitutive FGFR3 activity. In some embodiments, constitutive FGFR3 activity is ligand-dependent FGFR3 constitutive activity. In some embodiments, constitutive FGFR3 activity is ligand-independent constitutive FGFR3 activity.

In some embodiments, the FGFR3 antagonist inhibits FGFR3 comprising a mutation corresponding to FGFR3-IIIb^{R248C}. As used herein the term “comprising a mutation corresponding to FGFR3-IIIb^{R248C}” is understood to encompass FGFR3-IIIb^{R248C} and FGFR3-IIIc^{R248C}, as well as additional FGFR3 forms comprising an R to C mutation at a position corresponding to FGFR3-IIIb R248. One of ordinary skill in the art understands how to align FGFR3 sequences in order identify corresponding residues between respective FGFR3 sequences, e.g., aligning a FGFR3-IIIc sequence with a FGFR3-IIIb sequence to identify the position in FGFR3 corresponding R248 position in FGFR3-IIIb. In some embodiments, the FGFR3 antagonist inhibits FGFR3-IIIb^{R248C} and/or FGFR3-IIIc^{R248C}

In some embodiments, the FGFR3 antagonist inhibit FGFR3 comprising a mutation corresponding to FGFR3-IIIb^{K652E}. For convenience, the term “comprising a mutation corresponding to FGFR3-IIIb^{K652E}” is understood to encompass FGFR3-IIIb ^{K652E} and FGFR3-IIIc ^{K650E}, as well as additional FGFR3 forms comprising an K to E mutation at a position corresponding to FGFR3-IIIb K652. One of ordinary skill in the art understands how to align FGFR3 sequences in order identify corresponding residues between respective FGFR3 sequences, e.g., aligning a FGFR3-IIIc sequence with a FGFR3-IIIb sequence to identify the position in FGFR3 corresponding K652 position in FGFR3-IIIb. In some embodiments, the FGFR3 antagonist inhibits FGFR3-IIIb ^{K652E} and/or FGFR3-IIIc ^{K650E}.

In some embodiments, the FGFR3 antagonist inhibit FGFR3 comprising a mutation corresponding to FGFR3-IIIb^{S249C}. For convenience, the term “comprising a mutation corresponding to FGFR3-IIIb^{S249C}” is understood to encompass FGFR3-IIIb ^{S249C} and FGFR3-IIIc^{S249C}, as well as additional FGFR3 forms comprising an S to C mutation at a position corresponding to FGFR3-IIIb 5249. In some embodiments, the FGFR3 antagonist inhibits FGFR3-IIIb^{S249C} and/or FGFR3-IIIc^{S249C}.

In some embodiments, the FGFR3 antagonists inhibit FGFR3 comprising a mutation corresponding to FGFR3-IIIb^{G372C}. For convenience, the term “comprising a mutation corresponding to FGFR3-IIIb^{G372C}” is understood to encompass FGFR3-IIIb ^{G372C} and FGFR3-IIIc ^{G370C}, as well as additional FGFR3 forms comprising a G to C mutation at a position corresponding to FGFR3-IIIb G372. In some embodiments, the FGFR3 antagonist inhibits FGFR3-IIIb^{G372C} and/or FGFR3-IIIc ^{G370C}.

In some embodiments, the FGFR3 antagonists inhibit FGFR3 comprising a mutation corresponding to FGFR3-IIIb^{Y375C}. For convenience, the term “comprising a mutation corresponding to FGFR3-IIIb^{Y375C}” is understood to encompass FGFR3-IIIb^{Y375C} and FGFR3-IIIc^{Y373C}, as well as additional FGFR3 forms comprising an S to C mutation at a position corresponding to FGFR3-IIIb S249. In some embodiments, the FGFR3 antagonist inhibits FGFR3-IIIb^{Y375C} and/or FGFR3-IIIc^{Y373C}.

In some embodiments, the FGFR3 antagonist (a) FGFR3-IIIb^{K652E} and (b) one or more of FGFR3-IIIb^{R248C}, FGFR3-IIIb^{Y375C}, FGFR3-IIIb^{S249C}, and FGFR3IIIb ^{G372C}. In some embodiments, the FGFR3 antagonists inhibit (a) FGFR3-IIIc^{K650E} and (b) one or more of FGFR3-IIIc^{R248C}, FGFR3-IIIc^{Y373C}, FGFR3-IIIc^{S249C}, and FGFR3IIIc ^{G370C}. In some embodiments, the FGFR3 antagonists inhibit (a) FGFR3-IIIb^{R248C} and (b) one or more of FGFR3-IIIb^{K652E}, FGFR3-IIIb^{Y375C}, FGFR3-IIIb^{S249C}, and FGFR3-IIIb^{G372C}. In some embodiments, the FGFR3 antagonists inhibit (a) FGFR3-IIIc^{R248C} and (b) one or more of FGFR3-IIIc^{K650E}, FGFR3-IIIc^{Y373C}, FGFR3-IIIc^{S249C}, and FGFR3-IIIc^{G370C}. In some embodiments, the FGFR3 antagonists inhibit (a) FGFR3-IIIb^{G372C} and (b) one or more of FGFR3-IIIb^{K652E}, FGFR3-IIIb^{Y375C}, FGFR3-IIIb^{S249C}, and FGFR3-IIIb^{R248C}. In some embodiments, the FGFR3 antagonists inhibit (a) FGFR3-IIIc^{G370C} and (b) one or more of FGFR3-IIIc^{K650E}, FGFR3-IIIc^{Y373C}, FGFR3-IIIc^{S249C}, and FGFR3-IIIc^{R248C}. In some embodiments, the FGFR3 antagonists inhibit FGFR3-IIIb^{R248C}, FGFR3-IIIb^{K652E}, FGFR3-IIIb^{Y375C}, FGFR3-IIIb ^{S249C}, and FGFR3-IIIb ^{G372C}. In some embodiments, the FGFR3 antagonists inhibit FGFR3-IIIc^{R248C}, FGFR3-IIIc^{K650E}, FGFR3-IIIc^{Y373C}, FGFR3-IIIc ^{S249C}, and FGFR3-IIIc ^{G370C}.

### A. Antibodies

In some embodiments, the FGFR3 antagonist is an anti-FGFR3 antibody. In some embodiments, the FGFR3 antibodies is an isolated antibody that bind to FGFR3. In some embodiments, an antibody is humanized. In some embodiments, an anti-FGFR3 antibody according to any of the above embodiments is a monoclonal antibody, including a chimeric, humanized or human antibody. In some embodiments, an anti-FGFR3 antibody is an antibody fragment, e.g., a Fv, Fab, Fab′, scFv, diabody, or F(ab′)₂ fragment. In another embodiment, the antibody is a full length antibody, e.g., an intact IgG1″ antibody or other antibody class or isotype as defined herein.

In some embodiments, the anti-FGFR3 antibody is an isolated anti-FGFR3 antibody, wherein a full length IgG form of the antibody binds human FGFR3 with a Kd of 1×10⁻⁷ or stronger. As is well-established in the art, binding affinity of a ligand to its receptor can be determined using any of a variety of assays, and expressed in terms of a variety of quantitative values. Accordingly, in one embodiment, the binding affinity is expressed as Kd values and reflects intrinsic binding affinity (e.g., with minimized avidity effects). Generally and preferably, binding affinity is measured in vitro, whether in a cell-free or cell-associated setting. Any of a number of assays known in the art, including those described herein, can be used to obtain binding affinity measurements, including, for example, Biacore, radioimmunoassay (RIA), and ELISA. In some embodiments, the full length IgG form of the antibody binds human FGFR3 with a Kd of 1×10⁻⁸ or stronger, with a Kd of 1×10⁻⁹ or stronger, or with a Kd of 1×10⁻¹⁰ or stronger.

Generally, the anti-FGFR3 antibodies are antagonist antibodies. Thus, in some embodiments, the anti-FGFR3 antibodies inhibit FGFR3 activity (e.g., FGFR3-IIIb and/or FGFR3-IIIc activity). In some embodiments, the anti-FGFR3 antibody (generally in bivalent form) does not possess substantial FGFR3 agonist function. In some embodiments, the anti-FGFR3 antagonist antibody (generally in bivalent form) possesses little or no FGFR3 agonist function. In one embodiment, an antibody (generally in bivalent form) does not exhibit an FGFR3 agonist activity level that is above background level that is of statistical significance.

In some embodiments, binding of the antibody to a FGFR3 may inhibit dimerization of the receptor with another unit of the receptor, whereby activation of the receptor is inhibited (due, at least in part, to a lack of receptor dimerization). Inhibition can be direct or indirect.

In some embodiments, the anti-FGFR3 antibodies are anti-FGFR3 antibodies that do not possess substantial apoptotic activity (e.g., does not induce apoptosis of a cell, e.g., a transitional cell carcinoma cell or a multiple myeloma cell, such as a multiple myeloma cell comprising a FGFR3 translocation, such as a t(4; 14) translocation). In some embodiments, the anti-FGFR3 antibody possesses little or no apoptotic function. In some embodiment, the FGFR3 antibodies do not exhibit apoptotic function that is above background level that is of statistical significance.

In some embodiments, the anti-FGFR3 antibodies are anti-FGFR3 antibodies that do not induce substantial FGFR3 down-regulation. In some embodiments, the anti-FGFR3 antibody induces little or no receptor down-regulation. In some embodiment, the FGFR3 antibodies do not induce receptor down-regulation that is above background level that is of statistical significance.

In some embodiments, the anti-FGFR3 antibodies are anti-FGFR3 antibodies that possess effector function. In one embodiment, the effector function comprises antibody-dependent cell-mediated cytotoxicity (ADCC). In one embodiment, the anti-FGFR3 antibody (in some embodiments, a naked anti-FGFR3 antibody) is capable of killing a cell, in some embodiments, a multiple myeloma cells (e.g., multiple myeloma cells comprising a translocation, e.g., a t(4; 14) translocation). In some embodiments, the anti-FGFR3 antibody is capable of killing a cell that expresses about 10,000 FGFR3 molecules per cell or more (such as about 11,000, about 12,000, about 13,000, about 14,000, about 15,000, about 16,000, about 17,000, about 18,000 or more FGFR3 molecules per cell). In other embodiments, the cell expresses about 2000, about 3000, about 4000, about 5000, about 6000, about 7000, about 8000, or more FGFR3 molecules per cell.

In some embodiments, the anti-FGFR3 antibody is an isolated anti-FGFR3 antibody comprising: (a) at least one, two, three, four, or five hypervariable region (HVR) sequences selected from: (i) HVR-L1 comprising sequence A1-A11, wherein A1-A11 is RASQDVDTSLA (SEQ ID NO:87), (ii) HVR-L2 comprising sequence B1-B7, wherein B1-B7 is SASFLYS (SEQ ID NO:88), (iii) HVR-L3 comprising sequence C1-C9, wherein C1-C9 is QQSTGHPQT (SEQ ID NO:89), (iv) HVR-H1 comprising sequence D1-D10, wherein D1-D10 is GFTFTSTGIS (SEQ ID NO:84), (v) HVR-H2 comprising sequence E1-E18, wherein E1-E18 is GRIYPTSGSTNYADSVKG (SEQ ID NO:85), and (vi) HVR-H3 comprising sequence F1-F20, wherein F1-F20 is ARTYGIYDLYVDYTEYVMDY (SEQ ID NO:86); and (b) at least one variant HVR, where the variant HVR sequence comprises modification of at least one residue (at least two residues, at least three or more residues) of the sequence depicted in SEQ ID NOS: 1-18, 48-131 and 140-145. The modification desirably is a substitution, insertion, or deletion.

In some embodiments, a HVR-L1 variant comprises 1-6 (1, 2, 3, 4, 5, or 6) substitutions in any combination of the following positions: A5 (V or D), A6 (V or I), A7 (D, E or S), A8 (T or I), A9 (A or S) and A10 (V or L). In some embodiments, a HVR-L2 variant comprises 1-2 (1 or 2) substitutions in any combination of the following positions: B1 (S or G), B4 (F or S or T) and B6 (A or Y). In some embodiments, a HVR-L3 variant comprises 1-6 (1, 2, 3, 4, 5, or 6) substitutions in any combination of the following positions: C3 (G or S or T), C4 (T or Y or A), C5 (G or S or T or A), C6 (A or H or D or T or N), C7 (Q or P or S), and C8 (S or Y or L or P or Q). In some embodiment, a HVR-H1 variant comprises 1-3 (1, 2, or 3) substitutions in any combination of the following positions: D3 (S or T), D5 (W or Y or S or T), D6 (S or G or T). In some embodiment, a HVR-H2 variant comprises 1-6 (1, 2, 3, 4, 5, or 6) substitutions in any combination of the following positions: E2 (R or S), E6 (Y or A or L or S or T), E7 (A or Q or D or G or Y or S or N or F), E8 (A or D or G), E9 (T or S), E10 (K or F or T or S), E11 (Y or H or N or I).

In one embodiment, the invention provides an isolated anti-FGFR3 antibody comprising: (a) at least one, two, three, four, or five hypervariable region (HVR) sequences selected from: (i) HVR-L1 comprising sequence RASQX₁X₂X₃X₄X₅X₆A, wherein X₁ is V or D, X₂ is V or I, X₃ is D, E or S, X₄ is T or I, X₅ is A or S, and X₆ is V or L (SEQ ID NO:146), (ii) HVR-L2 comprising sequence X₁ASFLX₂S wherein X₁ is S or G and X₂ is A or Y (SEQ ID NO:147), (iii) HVR-L3 comprising sequence QQX₁X₂X₃X₄X₅X₆T, wherein X₁ is G, S or T, X₂ is T, Y or A, X₃ is G, S, T, or A, X₄ is A, H, D, T, or N, X₅ is Q, P or S, X₆ is S, Y, L, P or Q (SEQ ID NO:148), (iv) HVR-H1 comprising sequence GFX₁FX₂X₃TGIS, wherein X₁ is S or T, X₂ is W, Y, S or T, X₃ is S, G, or T (SEQ ID NO:149), (v) HVR-H2 comprising sequence GRIYPX₁X₂X₃X₄X₅X₆YADSVKG, wherein X₁ is Y, A, L, S, or T, X₂ is A, Q, D, G, Y, S, N or F, X₃ is A, D, or G, X₄ is T or S, X₅ is K, F, T, or S, X₆ is Y, H, N or I (SEQ ID NO:150), and (vi) HVR-H3 comprising sequence ARTYGIYDLYVDYTEYVMDY (SEQ ID NO:151).

In some embodiments, HVR-L1 comprises sequence RASQX₁VX₂X₃X₄VA, wherein X₁ is V or D, X₂ is D, E or S, X₃ is T or I, X₄ is A or S (SEQ ID NO:152). In some embodiments, HVR-L3 comprises sequence QQX₁X₂X₃X₄X₅X₆T, wherein X₁ is S, G, or T, X₂ is Y, T, or A, X₃ is T or G, X₄ is T, H or N, X₅ is P or S, X₆ is P, Q, Y, or L (SEQ ID NO:153). In some embodiments, HVR-H2 comprises sequence GRIYPX₁X₂GSTX₃YADSVKG, wherein X₁ is T or L, X₂ is N, Y, S, G, A, or Q; X₃ is N or H (SEQ ID NO:154).

In another embodiment, an isolated anti-FGFR3 antibody comprises one, two, three, four, five, or six HVRs, where each HVR comprises, consists, or consists essentially of a sequence selected from SEQ ID NOS:1-18, 48-131 and 140-145, and where SEQ ID NO:1, 7, 13, 48, 54, 60, 66, 72, 78, 84, 90, 96, 102, 108, 114, 120, 126 or 143 corresponds to an HVR-H1, SEQ ID NO:2, 8, 14, 49, 55, 61, 67, 73, 79, 85, 91, 97, 103, 109, 115, 121, 127 or 144 corresponds to an HVR-H2, SEQ ID NO:3, 9, 15, 50, 56, 62, 68, 74, 80, 86, 92, 98, 104, 110, 116, 122, 128 or 145 corresponds to an HVR-H3, SEQ ID NO:4, 10, 16, 51, 57, 63, 69, 75, 81, 87, 93, 99, 105, 111, 117, 123, 129 or 140 corresponds to an HVR-L1, SEQ ID NO:5, 11, 17, 52, 58, 64, 70, 76, 82, 88, 94, 100, 106, 112, 118, 124, 130 or 141 corresponds to an HVR-L2, and SEQ ID NO:6, 12, 18, 53, 59, 65, 71, 77, 83, 89, 95, 101, 107, 113, 119, 125, 131 or 142 corresponds to an HVR-L3.

In one embodiment, an anti-FGFR3 antibody comprises a heavy chain variable region comprising HVR-H1, HVR-H2, HVR-H3, wherein each, in order, comprises SEQ ID NO:1, 2, 3, and/or a light chain variable region comprising HVR-L1, HVR-L2, and HVR-L3, where each, in order, contains SEQ ID NO: 4, 5, 6.

In another embodiment, an anti-FGFR3 antibody comprises a heavy chain variable region comprising HVR-H1, HVR-H2, HVR-H3, wherein each, in order, comprises SEQ ID NO:7, 8, 9, and/or a light chain variable region comprising HVR-L1, HVR-L2, and HVR-L3, where each, in order, comprises SEQ ID NO: 10, 11, 12.

In another embodiment, an anti-FGFR3 antibody comprises a heavy chain variable region comprising HVR-H1, HVR-H2, HVR-H3, where each, in order, comprises SEQ ID NO:13, 14, 15, and/or a light chain variable region comprising HVR-L1, HVR-L2, and HVR-L3, where each, in order, comprises SEQ ID NO:16, 17, 18.

In another embodiment, an anti-FGFR3 antibody comprises a heavy chain variable region comprising HVR-H1, HVR-H2, HVR-H3, where each, in order, comprises SEQ ID NO: 48, 49, 50, and/or a light chain variable region HVR-L1, HVR-L2, and HVR-L3, where each, in order, comprises SEQ ID NO: 51, 52, 53.

In another embodiment, an anti-FGFR3 antibody comprises a heavy chain variable region comprising HVR-H1, HVR-H2, HVR-H3, where each, in order, comprises SEQ ID NO: 54, 55, 56, and/or a light chain variable region comprising HVR-L1, HVR-L2, and HVR-L3, where each, in order, comprises SEQ ID NO: 57, 58, 59.

In another embodiment, an anti-FGFR3 antibody comprises a heavy chain variable region comprising HVR-H1, HVR-H2, HVR-H3, where each, in order, comprises SEQ ID NO:60, 61, 62, 63, and/or a light chain variable region comprising HVR-L1, HVR-L2, and HVR-L3, where each, in order, comprises SEQ ID NO: 63, 64, 65.

In another embodiment, an anti-FGFR3 antibody comprises a heavy chain variable region comprising HVR-H1, HVR-H2, HVR-H3, where each, in order, comprises SEQ ID NO:66, 67, 68, and/or a light chain variable region comprising HVR-L1, HVR-L2, and HVR-L3, where each, in order, comprises SEQ ID NO: 69, 70, 71.

In another embodiment, an anti-FGFR3 antibody comprises a heavy chain variable region comprising HVR-H1, HVR-H2, HVR-H3, where each, in order, comprises SEQ ID NO:72, 73, 74, and/or a light chain variable region comprising HVR-L1, HVR-L2, and HVR-L3, where each, in order, comprises SEQ ID NO: 75, 76, 77.

In another embodiment, an anti-FGFR3 antibody comprises a heavy chain variable region comprising HVR-H1, HVR-H2, HVR-H3, where each, in order, comprises SEQ ID NO:78, 79 80, and/or a light chain variable region comprising HVR-L1, HVR-L2, and HVR-L3, where each, in order, comprises SEQ ID NO:81, 82, 83.

In another embodiment, an anti-FGFR3 antibody comprises a heavy chain variable region comprising HVR-H1, HVR-H2, HVR-H3, where each, in order, comprises SEQ ID NO: 84, 85, 86, and/or a light chain variable region comprising HVR-L1, HVR-L2, and HVR-L3, where each, in order, comprises SEQ ID NO:87, 88, 89.

In another embodiment, an anti-FGFR3 antibody comprises a heavy chain variable region comprising HVR-H1, HVR-H2, HVR-H3, where each, in order, comprises SEQ ID NO: 90, 91, 92, and/or a light chain variable region comprising HVR-L1, HVR-L2, and HVR-L3, where each, in order, comprises SEQ ID NO:93, 94, 95.

In another embodiment, an anti-FGFR3 antibody comprises a heavy chain variable region comprising HVR-H1, HVR-H2, HVR-H3, where each, in order, comprises SEQ ID NO: 96, 97, 98, and/or a light chain variable region comprising HVR-L1, HVR-L2, and HVR-L3, where each, in order, comprises SEQ ID NO: 99, 100, 101.

In another embodiment, an anti-FGFR3 antibody comprises a heavy chain variable region comprising HVR-H1, HVR-H2, HVR-H3, where each, in order, comprises SEQ ID NO: 102, 103, 104, and/or a light chain variable region comprising HVR-L1, HVR-L2, and HVR-L3, where each, in order, comprises SEQ ID NO: 105, 106, 107.

In another embodiment, an anti-FGFR3 antibody comprises a heavy chain variable region comprising HVR-H1, HVR-H2, HVR-H3, where each, in order, comprises SEQ ID NO: 108, 109, 110, and/or a light chain variable region comprising HVR-L1, HVR-L2, and HVR-L3, where each, in order, comprises SEQ ID NO: 111, 112, 113.

In another embodiment, an anti-FGFR3 antibody comprises a heavy chain variable region comprising HVR-H1, HVR-H2, HVR-H3, where each, in order, comprises SEQ ID NO:114, 115, 116, and/or a light chain variable region comprising HVR-L1, HVR-L2, and HVR-L3, where each, in order, comprises SEQ ID NO:117, 118, 119.

In another embodiment, an anti-FGFR3 antibody comprises a heavy chain variable region comprising HVR-H1, HVR-H2, HVR-H3, where each, in order, comprises SEQ ID NO:120, 121, 122, and/or a light chain variable region comprising HVR-L1, HVR-L2, and HVR-L3, where each, in order, comprises SEQ ID NO: 123, 124, 125.

In another embodiment, an anti-FGFR3 antibody comprises a heavy chain variable region comprising HVR-H1, HVR-H2, HVR-H3, where each, in order, comprises SEQ ID NO:126, 127, 128, and/or a light chain variable region comprising HVR-L1, HVR-L2, and HVR-L3, where each, in order, comprises SEQ ID NO:129, 130, 131.

In another embodiment, an anti-FGFR3 antibody comprises a heavy chain variable region comprising HVR-H1, HVR-H2, HVR-H3, where each, in order, comprises SEQ ID NO:143, 144, 145, and/or a light chain variable region comprising HVR-L1, HVR-L2, and HVR-L3, where each, in order, comprises SEQ ID NO:140, 141, 142.

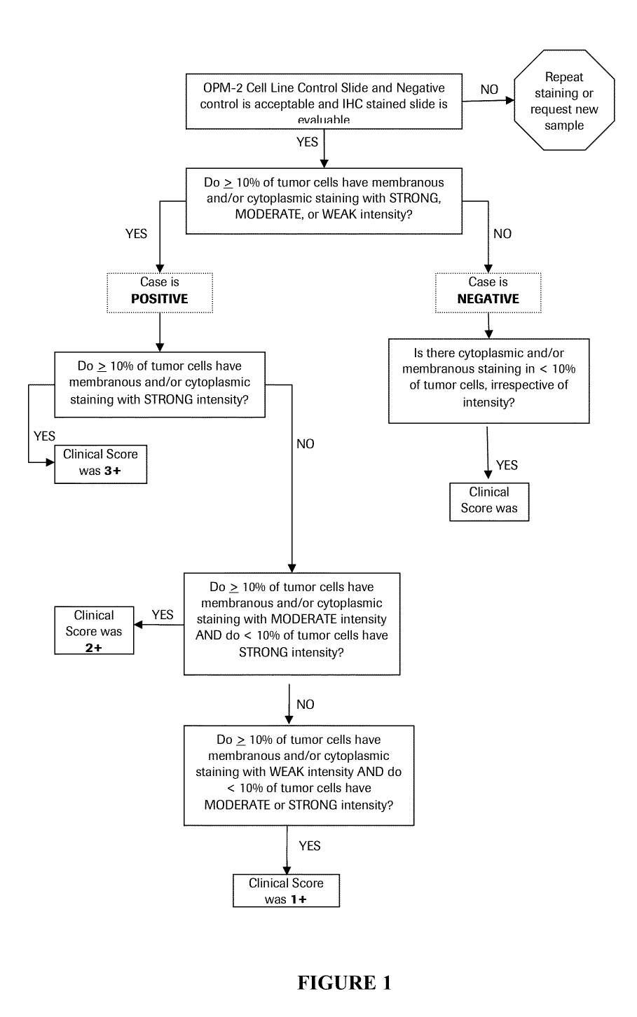

The amino acid sequences of SEQ ID NOs:1-18, 48-131 and 140-145 are numbered with respect to individual HVR (i.e., H1, H2 or H3) as indicated in FIG. 1, the numbering being consistent with the Kabat numbering system as described below.

In another embodiment, an anti-FGFR3 antibody comprises a heavy chain variable region comprising SEQ ID NO:132 and a light chain variable region. In another embodiment, an anti-FGFR3 antibody comprises a light chain variable region comprising SEQ ID NO: 133, and a heavy chain variable region. In another embodiment, an anti-FGFR3 antibody comprises a heavy chain variable region comprising SEQ ID NO:132 and a light chain variable region comprising SEQ ID NO:133.

In another embodiment, an anti-FGFR3 antibody comprises a heavy chain variable region comprising SEQ ID NO:134 and a light chain variable region. In another embodiment, an anti-FGFR3 antibody comprises a light chain variable region comprising SEQ ID NO: 135, and a heavy chain variable region. In another embodiment, an anti-FGFR3 antibody comprises a heavy chain variable region comprising SEQ ID NO:134 and a light chain variable region comprising SEQ ID NO:135. The anti-FGFR3 antibody R3MAb as described herein is an anti-FGFR3 antibody comprises a heavy chain variable region comprising SEQ ID NO:134 and a light chain variable region comprising SEQ ID NO:135. Specifically provided herein is the isolated anti-FGFR3 antibody and methods of using the isolated anti-FGFR3 antibody (including in the treat of a disease or disorder such as cancer) comprising a heavy chain variable region comprising SEQ ID NO: 134 and/or a light chain variable region comprising SEQ ID NO:135.

In another embodiment, an anti-FGFR3 antibody comprises a heavy chain variable region comprising SEQ ID NO:136 and a light chain variable region. In another embodiment, an anti-FGFR3 antibody comprises a light chain variable region comprising SEQ ID NO: 137, and a heavy chain variable region. In another embodiment, an anti-FGFR3 antibody comprises a heavy chain variable region comprising SEQ ID NO:136 and a light chain variable region comprising SEQ ID NO:137.

In another embodiment, an anti-FGFR3 antibody comprises a heavy chain variable region comprising SEQ ID NO:138 and a light chain variable region. In another embodiment, an anti-FGFR3 antibody comprises a light chain variable region comprising SEQ ID NO: 139, and a heavy chain variable region. In another embodiment, an anti-FGFR3 antibody comprises a heavy chain variable region comprising SEQ ID NO:138 and a light chain variable region comprising SEQ ID NO:139.

In one embodiment, the invention provides an anti-FGFR3 antibody comprising: at least one, two, three, four, five, and/or six hypervariable region (HVR) sequences selected from the group consisting of: (a) HVR-L1 comprising sequence SASSSVSYMH (SEQ ID NO:155), SASSSVSYMH (SEQ ID NO:156) or LASQTIGTWLA (SEQ ID NO:157), (b) HVR-L2 comprising sequence TWIYDTSILAS (SEQ ID NO:158), RWIYDTSKLAS (SEQ ID NO:159), or LLIYAATSLAD (SEQ ID NO:160), (c) HVR-L3 comprising sequence QQWTSNPLT (SEQ ID NO:161), QQWSSYPPT (SEQ ID NO:162), or QQLYSPPWT (SEQ ID NO:163), (d) HVR-H1 comprising sequence GYSFTDYNMY (SEQ ID NO:164), GYVFTHYNMY (SEQ ID NO:165), or GYAFTSYNMY (SEQ ID NO:166), (e) HVR-H2 comprising sequence IGYIEPYNGGTSYNQKFKG (SEQ ID NO:167), WIGYIEPYNGGTSYNQKFKG (SEQ ID NO:168), or WIGYIDPYIGGTSYNQKFKG (SEQ ID NO:169), and (f) HVR-H3 comprising sequence ASPNYYDSSPFAY (SEQ ID NO:170), ARGQGPDFDV (SEQ ID NO:171), or ARWGDYDVGAMDY (SEQ ID NO:172).

In one embodiment, the invention provides an anti-FGFR3 antibody comprising: at least one, two, three, four, five, and/or six hypervariable region (HVR) sequences selected from the group consisting of: (a) HVR-L1 comprising sequence SASSSVSYMH (SEQ ID NO:155), (b) HVR-L2 comprising sequence TWIYDTSILAS (SEQ ID NO:158), (c) HVR-L3 comprising sequence QQWTSNPLT (SEQ ID NO:161), (d) HVR-H1 comprising sequence GYSFTDYNMY (SEQ ID NO:164), (e) HVR-H2 comprising sequence IGYIEPYNGGTSYNQKFKG (SEQ ID NO:167), and (f) HVR-H3 comprising sequence ASPNYYDSSPFAY (SEQ ID NO:170).

In one embodiment, the invention provides an anti-FGFR3 antibody comprising: at least one, two, three, four, five, and/or six hypervariable region (HVR) sequences selected from the group consisting of: (a) HVR-L1 comprising sequence SASSSVSYMH (SEQ ID NO:156), (b) HVR-L2 comprising sequence RWIYDTSKLAS (SEQ ID NO: 159), (c) HVR-L3 comprising sequence QQWSSYPPT (SEQ ID NO:162), (d) HVR-H1 comprising sequence GYVFTHYNMY (SEQ ID NO:165), (e) HVR-H2 comprising sequence WIGYIEPYNGGTSYNQKFKG (SEQ ID NO:168), and (f) HVR-H3 comprising sequence ARGQGPDFDV (SEQ ID NO:171).

In one embodiment, the invention provides an anti-FGFR3 antibody comprising: at least one, two, three, four, five, and/or six hypervariable region (HVR) sequences selected from the group consisting of: (a) HVR-L1 comprising sequence LASQTIGTWLA (SEQ ID NO:157), (b) HVR-L2 comprising sequence LLIYAATSLAD (SEQ ID NO: 160), (c) HVR-L3 comprising sequence QQLYSPPWT (SEQ ID NO:163), (d) HVR-H1 comprising sequence GYAFTSYNMY (SEQ ID NO:166), (e) HVR-H2 comprising sequence WIGYIDPYIGGTSYNQKFKG (SEQ ID NO:169), and (f) HVR-H3 comprising sequence ARWGDYDVGAMDY (SEQ ID NO:172).

In one embodiment, the invention provides an anti-FGFR3 antibody comprising (a) a light chain comprising (i) HVR-L1 comprising sequence SASSSVSYMH (SEQ ID NO:155); (ii) HVR-L2 comprising sequence TWIYDTSILAS (SEQ ID NO:158); and (iii) HVR-L3 comprising sequence QQWTSNPLT (SEQ ID NO:161); and/or (b) a heavy chain comprising (i) HVR-H1 comprising sequence GYSFTDYNMY (SEQ ID NO:164); (ii) HVR-H2 comprising sequence IGYIEPYNGGTSYNQKFKG (SEQ ID NO:167); and (iii) HVR-H3 comprising sequence ASPNYYDSSPFAY (SEQ ID NO:170).

In one embodiment, the invention provides an anti-FGFR3 antibody comprising (a) a light chain comprising (i) HVR-L1 comprising sequence SASSSVSYMH (SEQ ID NO:156); (ii) HVR-L2 comprising sequence RWIYDTSKLAS (SEQ ID NO: 159); and (iii) HVR-L3 comprising sequence QQWSSYPPT (SEQ ID NO:162); and/or (b) a heavy chain comprising (i) HVR-H1 comprising sequence GYVFTHYNMY (SEQ ID NO:165); (ii) HVR-H2 comprising sequence WIGYIEPYNGGTSYNQKFKG (SEQ ID NO:168); and (iii) HVR-H3 comprising sequence ARGQGPDFDV (SEQ ID NO:171).

In one embodiment, the invention provides an anti-FGFR3 antibody comprising (a) a light chain comprising (i) HVR-L1 comprising sequence LASQTIGTWLA (SEQ ID NO:157); (ii) HVR-L2 comprising sequence LLIYAATSLAD (SEQ ID NO: 160); and (iii) HVR-L3 comprising sequence QQLYSPPWT (SEQ ID NO:163); and/or (b) a heavy chain comprising (i) HVR-H1 comprising sequence GYAFTSYNMY (SEQ ID NO:166); (ii) HVR-H2 comprising sequence WIGYIDPYIGGTSYNQKFKG (SEQ ID NO:169); and (iii) HVR-H3 comprising sequence ARWGDYDVGAMDY (SEQ ID NO: 172). Some embodiments of antibodies comprise a light chain variable domain of humanized 4D5 antibody (huMAb4D5-8) (HERCEPTIN®, Genentech, Inc., South San Francisco, Calif., USA) (also referred to in U.S. Pat. No. 6,407,213 and Lee et al., J. Mol. Biol. (2004), 340 (5):1073-1093) as depicted in SEQ ID NO:173 below:

1 Asp Ile Gln Met Thr Gln Ser Pro Ser Ser Leu Ser Ala Ser Val Gly Asp Arg Val Thr Ile Thr Cys Arg Ala Ser Gln Asp Val Asn Thr Ala Val Ala Trp Tyr Gln Gln Lys Pro Gly Lys Ala Pro Lys Leu Leu Ile Tyr Ser Ala Ser Phe Leu Tyr Ser Gly Val Pro Ser Arg Phe Ser Gly Ser Arg Ser Gly Thr Asp Phe Thr Leu Thr Ile Ser Ser Leu Gln Pro Glu Asp Phe Ala Thr Tyr Tyr Cys Gln Gln His Tyr Thr Thr Pro Pro Thr Phe Gly Gln Gly Thr Lys Val Glu Ile Lys 107 (SEQ ID NO:173) (HVR residues are underlined)

In one embodiment, the huMAb4D5-8 light chain variable domain sequence is modified at one or more of positions 30, 66, and 91 (Asn, Arg, and His as indicated in bold/italics above, respectively). In a particular embodiment, the modified huMAb4D5-8 sequence comprises Ser in position 30, Gly in position 66, and/or Ser in position 91. Accordingly, in one embodiment, an antibody comprises a light chain variable domain comprising the sequence depicted in SEQ ID NO:174 below:

1 Asp Ile Gln Met Thr Gln Ser Pro Ser Ser Leu Ser Ala Ser Val Gly Asp Arg Val Thr Ile Thr Cys Arg Ala Ser Gln Asp Val Ser Thr Ala Val Ala Trp Tyr Gln Gln Lys Pro Gly Lys Ala Pro Lys Leu Leu Ile Tyr Ser Ala Ser Phe Leu Tyr Ser Gly Val Pro Ser Arg Phe Ser Gly Ser Gly Ser Gly Thr Asp Phe Thr Leu Thr Ile Ser Ser Leu Gln Pro Glu Asp Phe Ala Thr Tyr Tyr Cys Gln Gln Ser Tyr Thr Thr Pro Pro Thr Phe Gly Gln Gly Thr Lys Val Glu Ile Lys 107 (SEQ ID NO:174) (HVR residues are underlined)

Substituted residues with respect to huMAb4D5-8 are indicated in bold/italics.

Antibodies can comprise any suitable framework variable domain sequence, provided binding activity to FGFR3 is substantially retained. For example, in some embodiments, antibodies comprise a human subgroup III heavy chain framework consensus sequence. In one embodiment of these antibodies, the framework consensus sequence comprises a substitution at position 71, 73, and/or 78. In some embodiments of these antibodies, position 71 is A, 73 is T and/or 78 is A. In one embodiment, these antibodies comprise heavy chain variable domain framework sequences of huMAb4D5-8 (HERCEPTIN®, Genentech, Inc., South San Francisco, Calif., USA) (also referred to in U.S. Pat. Nos. 6,407,213 & 5,821,337, and Lee et al., J. Mol. Biol. (2004), 340(5):1073-1093). In one embodiment, these antibodies further comprise a human κI light chain framework consensus sequence. In a particular embodiment, these antibodies comprise light chain HVR sequences of huMAb4D5-8 as described in U.S. Pat. Nos. 6,407,213 & 5,821,337.) In one embodiment, these antibodies comprise light chain variable domain sequences of huMAb4D5-8 (HERCEPTIN®, Genentech, Inc., South San Francisco, Calif., USA) (also referred to in U.S. Pat. Nos. 6,407,213 & 5,821,337, and Lee et al., J. Mol. Biol. (2004), 340 (5):1073-1093).

In one embodiment, an antibody comprises a heavy chain variable domain comprising HVR H1, H2, and H3 sequences are SEQ ID NOS:13, 14 and/or 15, respectively. In another embodiment, the HVR H1, H2, and H3 sequences are SEQ ID NOS:48, 49 and/or 50, respectively. In yet another embodiment, the HVR H1, H2, and H3 sequences are SEQ ID NOS:84, 85, and/or 86, respectively. In a further embodiment, the HVR H1, H2, and H3 sequences are SEQ ID NOS:108, 109, and/or 110, respectively.

In a particular embodiment, an antibody comprises a light chain variable domain, and HVR L1, L2, and L3 sequences are SEQ ID NOS:16, 17, and/or 18, respectively. In another embodiment, an antibody comprises a light chain variable domain, the HVR L1, L2, and L3 sequences are SEQ ID NOS:51, 52 and/or 53, respectively. In an additional embodiment, an antibody comprises a light chain variable domain, and HVR L1, L2, and L3 sequences are SEQ ID NOS:87, 88 and/or 89, respectively. In yet another embodiment, an antibody comprises a light chain variable domain, the HVR L1, L2, and L3 sequences are SEQ ID NOS:111, 112, and/or 113, respectively.

In another embodiment, an antibody comprises a heavy chain variable domain comprising the sequence of SEQ ID NO: 132 and/or a light chain variable domain comprising the sequence of SEQ ID NO:133. In another embodiment, an antibody comprises a heavy chain variable domain comprising the sequence of SEQ ID NO: 134 and/or a light chain variable domain comprising the sequence of SEQ ID NO:135. In another embodiment, an antibody comprises a heavy chain variable domain comprising the sequence of SEQ ID NO:136 and/or a light chain variable domain comprising the sequence of SEQ ID NO:137. In another embodiment, an antibody comprises a heavy chain variable domain comprising the sequence of SEQ ID NO:138 and/or a light chain variable domain comprising the sequence of SEQ ID NO:139.

In one embodiment, the invention provides an anti-FGFR3 antibody that binds a polypeptide comprising, consisting essentially of or consisting of the following amino acid sequence: LAVPAANTVRFRCPA (SEQ ID NO:179) and/or SDVEFHCKVYSDAQP (SEQ ID NO:180).

In some embodiments, the antibody binds a polypeptide comprising, consisting essentially of or consisting of amino acid numbers 164-178 and/or 269-283 of the mature human FGFR3 amino acid sequence.

In one embodiment, an anti-FGFR3 antibody specifically binds an amino acid sequence having at least 50%, 60%, 70%, 80%, 90%, 95%, 98% sequence identity or similarity with the sequence LAVPAANTVRFRCPA (SEQ ID NO:179) and/or SDVEFHCKVYSDAQP (SEQ ID NO:180). In one embodiment, the anti-FGFR3 antibody of the present invention binds to at least one, two, three, four, or any number up to all of residues 154, 155, 158, 159, 161, 162, 163, 164, 165, 166, 167, 168, 169, 170, 171, 172, 173, 174, 175, 177, 202, 205, 207, 210, 212, 214, 216, 217, 241, 246, 247, 248, 278, 279, 280, 281, 282, 283, 314,

In a further embodiment, an anti-FGFR3 antibody according to any of the above embodiments may incorporate any of the features, singly or in combination, as described in Sections below:

1. Antibody Affinity

In certain embodiments, an antibody provided herein has a dissociation constant (Kd) of ≦1 μM. In one embodiment, Kd is measured by a radiolabeled antigen binding assay (RIA) performed with the Fab version of an antibody of interest and its antigen as described by the following assay. Solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881 (1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using surface plasmon resonance assays using a BIACORE®-2000 or a BIACORE®-3000 (BIAcore, Inc., Piscataway, N.J.) at 25° C. with immobilized antigen CM5 chips at ˜10 response units (RU). Briefly, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio k_{off}/kₒₙ. See, e.g., Chen et al., J. Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

2. Antibody Fragments

In certain embodiments, an antibody provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthiin, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as production by recombinant host cells (e.g., E. coli or phage), as described herein.

3. Chimeric and Humanized Antibodies

In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing SDR (a-CDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

4. Human Antibodies

In certain embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HuMab® technology; U.S. Pat. No. 7,041,870 describing K-M MOUSE® technology, and U.S. Patent Application Publication No. US 2007/0061900, describing VelociMouse® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26 (4):265-268 (2006) (describing human-human hybridomas). Human hybridoma technology (Trioma technology) is also described in Vollmers and Brandlein, Histology and Histopathology, 20 (3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

5. Library-Derived Antibodies

Antibodies may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284 (1-2): 119-132 (2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

6. Multispecific Antibodies

In certain embodiments, an antibody provided herein is a multispecific antibody, e.g., a bispecific antibody. Multispecific antibodies are monoclonal antibodies that have binding specificities for at least two different sites. In certain embodiments, one of the binding specificities is for FGFR3 and the other is for any other antigen. In certain embodiments, bispecific antibodies may bind to two different epitopes of FGFR3. Bispecific antibodies may also be used to localize cytotoxic agents to cells which express FGFR3. Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g., Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g., US 2006/0025576A1).

The antibody or fragment herein also includes a “Dual Acting FAb” or “DAF” comprising an antigen binding site that binds to FGFR3 as well as another, different antigen (see, US 2008/0069820, for example).

7. Antibody Variants

a) Glycosylation Variants

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about +3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al., Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94 (4):680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

b) Fc Region Variants

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g., a substitution) at one or more amino acid positions.

In certain embodiments, the invention contemplates an antibody variant that possesses some but not all effector functions, which make it a desirable candidate for applications in which the half life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hemato FGFR3etic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S, and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18 (12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).) In certain embodiments, an antibody variant comprises an Fc region with one or more amino acid substitutions which improve ADCC, e.g., substitutions at positions 298, 333, and/or 334 of the Fc region (EU numbering of residues). In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826). See also Duncan & Winter, Nature 322:738-40 (1988); U.S. Pat. No. 5,648,260; U.S. Pat. No. 5,624,821; and WO 94/29351 concerning other examples of Fc region variants.

c) Cysteine Engineered Antibody Variants

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

### B. Immunoconjugates

Further provided herein are immunoconjugates comprising an anti-FGFR3 antibody herein conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In one embodiment, an immunoconjugate is an antibody-drug conjugate (ADC) in which an antibody is conjugated to one or more drugs, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In another embodiment, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes.

In another embodiment, an immunoconjugate comprises an antibody as described herein conjugated to a radioactive atom to form a radioconjugate. A variety of radioactive isotopes are available for the production of radioconjugates. Examples include At²¹¹, I¹³¹, I¹²⁵, Y⁹⁰, Re⁸⁶, Re⁸⁸, Sm⁵³, Bi²¹², P³², Pb²¹² and radioactive isotopes of Lu. When the radioconjugate is used for detection, it may comprise a radioactive atom for scintigraphic studies, for example tc⁹⁹ or I¹²³, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio)propionate (SPDP), succinimidyl-4-(N-maleimidomethyl)cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunuoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., U.S.A).

### C. Binding Polypeptides

Binding polypeptides are polypeptides that bind, preferably specifically, to FGFR3 as described herein. In some embodiments, the binding polypeptides are FGFR3 antagonists. Binding polypeptides may be chemically synthesized using known polypeptide synthesis methodology or may be prepared and purified using recombinant technology. Binding polypeptides are usually at least about 5 amino acids in length, alternatively at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids in length or more, wherein such binding polypeptides that are capable of binding, preferably specifically, to a target, FGFR3, as described herein. Binding polypeptides may be identified without undue experimentation using well known techniques. In this regard, it is noted that techniques for screening polypeptide libraries for binding polypeptides that are capable of specifically binding to a polypeptide target are well known in the art (see, e.g., U.S. Pat. Nos. 5,556,762, 5,750,373, 4,708,871, 4,833,092, 5,223,409, 5,403,484, 5,571,689, 5,663,143; PCT Publication Nos. WO 84/03506 and WO84/03564; Geysen et al., Proc. Natl. Acad. Sci. U.S.A., 81:3998-4002 (1984); Geysen et al., Proc. Natl. Acad. Sci. U.S.A., 82:178-182 (1985); Geysen et al., in Synthetic Peptides as Antigens, 130-149 (1986); Geysen et al., J. Immunol. Meth., 102:259-274 (1987); Schoofs et al., J. Immunol., 140:611-616 (1988), Cwirla, S. E. et al. (1990) Proc. Natl. Acad. Sci. USA, 87:6378; Lowman, H. B. et al. (1991) Biochemistry, 30:10832; Clackson, T. et al. (1991) Nature, 352: 624; Marks, J. D. et al. (1991), J. Mol. Biol., 222:581; Kang, A. S. et al. (1991) Proc. Natl. Acad. Sci. USA, 88:8363, and Smith, G. P. (1991) Current Opin. Biotechnol., 2:668).

In this regard, bacteriophage (phage) display is one well known technique which allows one to screen large polypeptide libraries to identify member(s) of those libraries which are capable of specifically binding to a target polypeptide, FGFR3. Phage display is a technique by which variant polypeptides are displayed as fusion proteins to the coat protein on the surface of bacteriophage particles (Scott, J. K. and Smith, G. P. (1990) Science, 249: 386). The utility of phage display lies in the fact that large libraries of selectively randomized protein variants (or randomly cloned cDNAs) can be rapidly and efficiently sorted for those sequences that bind to a target molecule with high affinity. Display of peptide (Cwirla, S. E. et al. (1990) Proc. Natl. Acad. Sci. USA, 87:6378) or protein (Lowman, H. B. et al. (1991) Biochemistry, 30:10832; Clackson, T. et al. (1991) Nature, 352: 624; Marks, J. D. et al. (1991), J. Mol. Biol., 222:581; Kang, A. S. et al. (1991) Proc. Natl. Acad. Sci. USA, 88:8363) libraries on phage have been used for screening millions of polypeptides or oligopeptides for ones with specific binding properties (Smith, G. P. (1991) Current Opin. Biotechnol., 2:668). Sorting phage libraries of random mutants requires a strategy for constructing and propagating a large number of variants, a procedure for affinity purification using the target receptor, and a means of evaluating the results of binding enrichments. U.S. Pat. Nos. 5,223,409, 5,403,484, 5,571,689, and 5,663,143.

Although most phage display methods have used filamentous phage, lambdoid phage display systems (WO 95/34683; U.S. Pat. No. 5,627,024), T4 phage display systems (Ren et al., Gene, 215: 439 (1998); Zhu et al., Cancer Research, 58 (15): 3209-3214 (1998); Jiang et al., Infection & Immunity, 65 (11): 4770-4777 (1997); Ren et al., Gene, 195 (2):303-311 (1997); Ren, Protein Sci., 5: 1833 (1996); Efimov et al., Virus Genes, 10: 173 (1995)) and T7 phage display systems (Smith and Scott, Methods in Enzymology, 217: 228-257 (1993); U.S. Pat. No. 5,766,905) are also known.

Additional improvements enhance the ability of display systems to screen peptide libraries for binding to selected target molecules and to display functional proteins with the potential of screening these proteins for desired properties. Combinatorial reaction devices for phage display reactions have been developed (WO 98/14277) and phage display libraries have been used to analyze and control bimolecular interactions (WO 98/20169; WO 98/20159) and properties of constrained helical peptides (WO 98/20036). WO 97/35196 describes a method of isolating an affinity ligand in which a phage display library is contacted with one solution in which the ligand will bind to a target molecule and a second solution in which the affinity ligand will not bind to the target molecule, to selectively isolate binding ligands. WO 97/46251 describes a method of biopanning a random phage display library with an affinity purified antibody and then isolating binding phage, followed by a micropanning process using microplate wells to isolate high affinity binding phage. The use of Staphlylococcus aureus protein A as an affinity tag has also been reported (Li et al. (1998) Mol. Biotech., 9:187). WO 97/47314 describes the use of substrate subtraction libraries to distinguish enzyme specificities using a combinatorial library which may be a phage display library. A method for selecting enzymes suitable for use in detergents using phage display is described in WO 97/09446. Additional methods of selecting specific binding proteins are described in U.S. Pat. Nos. 5,498,538, 5,432,018, and WO 98/15833.

Methods of generating peptide libraries and screening these libraries are also disclosed in U.S. Pat. Nos. 5,723,286, 5,432,018, 5,580,717, 5,427,908, 5,498,530, 5,770,434, 5,734,018, 5,698,426, 5,763,192, and 5,723,323.

### D. Binding Small Molecules

Provided herein are binding small molecules for use as a FGFR3 small molecule antagonist.

Binding small molecules are preferably organic molecules other than binding polypeptides or antibodies as defined herein that bind, preferably specifically, to FGFR3 as described herein. Binding organic small molecules may be identified and chemically synthesized using known methodology (see, e.g., PCT Publication Nos. WO00/00823 and WO00/39585). Binding organic small molecules are usually less than about 2000 daltons in size, alternatively less than about 1500, 750, 500, 250 or 200 daltons in size, wherein such organic small molecules that are capable of binding, preferably specifically, to a polypeptide as described herein may be identified without undue experimentation using well known techniques. In this regard, it is noted that techniques for screening organic small molecule libraries for molecules that are capable of binding to a polypeptide target are well known in the art (see, e.g., PCT Publication Nos. WO00/00823 and WO00/39585). Binding organic small molecules may be, for example, aldehydes, ketones, oximes, hydrazones, semicarbazones, carbazides, primary amines, secondary amines, tertiary amines, N-substituted hydrazines, hydrazides, alcohols, ethers, thiols, thioethers, disulfides, carboxylic acids, esters, amides, ureas, carbamates, carbonates, ketals, thioketals, acetals, thioacetals, aryl halides, aryl sulfonates, alkyl halides, alkyl sulfonates, aromatic compounds, heterocyclic compounds, anilines, alkenes, alkynes, diols, amino alcohols, oxazolidines, oxazolines, thiazolidines, thiazolines, enamines, sulfonamides, epoxides, aziridines, isocyanates, sulfonyl chlorides, diazo compounds, acid chlorides, or the like.

In some embodiments of any of the methods, the FGFR3 antagonist is Brivanib, Dovitinib (TKI-258), and/or HM-80871A.

### E. Antagonist Polynucleotides

Provided herein are polynucleotide antagonists. The polynucleotide may be an antisense nucleic acid and/or a ribozyme. The antisense nucleic acids comprise a sequence complementary to at least a portion of an RNA transcript of a FGFR3 gene. However, absolute complementarity, although preferred, is not required.

A sequence “complementary to at least a portion of an RNA,” referred to herein, means a sequence having sufficient complementarity to be able to hybridize with the RNA, forming a stable duplex; in the case of double stranded FGFR3 antisense nucleic acids, a single strand of the duplex DNA may thus be tested, or triplex formation may be assayed. The ability to hybridize will depend on both the degree of complementarity and the length of the antisense nucleic acid. Generally, the larger the hybridizing nucleic acid, the more base mismatches with an FGFR3RNA it may contain and still form a stable duplex (or triplex as the case may be). One skilled in the art can ascertain a tolerable degree of mismatch by use of standard procedures to determine the melting point of the hybridized complex.

Polynucleotides that are complementary to the 5′ end of the message, e.g., the 5′ untranslated sequence up to and including the AUG initiation codon, should work most efficiently at inhibiting translation. However, sequences complementary to the 3′ untranslated sequences of mRNAs have been shown to be effective at inhibiting translation of mRNAs as well. See generally, Wagner, R., 1994, Nature 372:333-335. Thus, oligonucleotides complementary to either the 5′- or 3′-non-translated, non-coding regions of the FGFR3 gene, could be used in an antisense approach to inhibit translation of endogenous FGFR3 mRNA. Polynucleotides complementary to the 5′ untranslated region of the mRNA should include the complement of the AUG start codon. Antisense polynucleotides complementary to mRNA coding regions are less efficient inhibitors of translation but could be used in accordance with the invention. Whether designed to hybridize to the 5′-, 3′- or coding region of FGFR3 mRNA, antisense nucleic acids should be at least six nucleotides in length, and are preferably oligonucleotides ranging from 6 to about 50 nucleotides in length. In specific embodiments the oligonucleotide is at least 10 nucleotides, at least 17 nucleotides, at least 25 nucleotides or at least 50 nucleotides.

In one embodiment, the FGFR3 antisense nucleic acid is produced intracellularly by transcription from an exogenous sequence. For example, a vector or a portion thereof, is transcribed, producing an antisense nucleic acid (RNA) of the FGFR3 gene. Such a vector would contain a sequence encoding the FGFR3 antisense nucleic acid. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others know in the art, used for replication and expression in vertebrate cells. Expression of the sequence encoding FGFR3, or fragments thereof, can be by any promoter known in the art to act in vertebrate, preferably human cells. Such promoters can be inducible or constitutive. Such promoters include, but are not limited to, the SV40 early promoter region (Bernoist and Chambon, Nature 29:304-310 (1981), the promoter contained in the 3′ long terminal repeat of Rous sarcoma virus (Yamamoto et al., Cell 22:787-797 (1980), the herpes thymidine promoter (Wagner et al., Proc. Natl. Acad. Sci. U.S.A. 78:1441-1445 (1981), the regulatory sequences of the metallothionein gene (Brinster, et al., Nature 296:39-42 (1982)), etc.

### F. Antibody and Binding Polypeptide Variants

In certain embodiments, amino acid sequence variants of the antibodies and/or the binding polypeptides provided herein are contemplated. For example, it may be desirable to improve the binding affinity and/or other biological properties of the antibody and/or binding polypeptide. Amino acid sequence variants of an antibody and/or binding polypeptides may be prepared by introducing appropriate modifications into the nucleotide sequence encoding the antibody and/or binding polypeptide, or by peptide synthesis. Such modifications include, for example, deletions from, and/or insertions into and/or substitutions of residues within the amino acid sequences of the antibody and/or binding polypeptide. Any combination of deletion, insertion, and substitution can be made to arrive at the final construct, provided that the final construct possesses the desired characteristics, e.g., target-binding.

In certain embodiments, antibody variants and/or binding polypeptide variants having one or more amino acid substitutions are provided. Sites of interest for substitutional mutagenesis include the HVRs and FRs. Conservative substitutions are shown in Table 1 under the heading of “conservative substitutions.” More substantial changes are provided in Table 1 under the heading of “exemplary substitutions,” and as further described below in reference to amino acid side chain classes. Amino acid substitutions may be introduced into an antibody and/or binding polypeptide of interest and the products screened for a desired activity, e.g., retained/improved antigen binding, decreased immunogenicity, or improved ADCC or CDC.

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g., binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or SDRs. In certain embodiments of the variant VH and VL sequences provided above, each HVR either is unaltered, or contains no more than one, two or three amino acid substitutions.

A useful method for identification of residues or regions of the antibody and/or the binding polypeptide that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact Points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

### G. Antibody and Binding Polypeptide Derivatives

In certain embodiments, an antibody and/or binding polypeptide provided herein may be further modified to contain additional nonproteinaceous moieties that are known in the art and readily available. The moieties suitable for derivatization of the antibody and/or binding polypeptide include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody and/or binding polypeptide may vary, and if more than one polymer are attached, they can be the same or different molecules. In general, the number and/or type of polymers used for derivatization can be determined based on considerations including, but not limited to, the particular properties or functions of the antibody and/or binding polypeptide to be improved, whether the antibody derivative and/or binding polypeptide derivative will be used in a therapy under defined conditions, etc.

In another embodiment, conjugates of an antibody and/or binding polypeptide to nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody and/or binding polypeptide-nonproteinaceous moiety are killed.

### IV. Recombinant Methods and Compositions

Antibodies and/or binding polypeptides may be produced using recombinant methods and compositions, e.g., as described in U.S. Pat. No. 4,816,567. In one embodiment, isolated nucleic acid encoding an anti-FGFR3 antibody. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid encoding the antibody and/or binding polypeptide are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g., a Chinese Hamster Ovary (CHO) cell or lymphoid cell (e.g., Y0, NS0, Sp20 cell). In one embodiment, a method of making an antibody such as an anti-FGFR3 antibody and/or binding polypeptide is provided, wherein the method comprises culturing a host cell comprising a nucleic acid encoding the antibody and/or binding polypeptide, as provided above, under conditions suitable for expression of the antibody and/or binding polypeptide, and optionally recovering the antibody and/or polypeptide from the host cell (or host cell culture medium).

For recombinant production of an antibody such as an anti-FGFR3 antibody and/or a binding polypeptide, nucleic acid encoding the antibody and/or the binding polypeptide, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody and/or glycosylated binding polypeptides are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production and/or binding polypeptide production, see, e.g., Yazaki and Wu, Methods in Molecular Biology, Vol. 248 (B. K. C. Lo, ed., Humana Press, Totowa, N.J.), pp. 255-268 (2003).

While the description relates primarily to production of antibodies and/or binding polypeptides by culturing cells transformed or transfected with a vector containing antibody- and binding polypeptide-encoding nucleic acid. It is, of course, contemplated that alternative methods, which are well known in the art, may be employed to prepare antibodies and/or binding polypeptides. For instance, the appropriate amino acid sequence, or portions thereof, may be produced by direct peptide synthesis using solid-phase techniques [see, e.g., Stewart et al., Solid-Phase Peptide Synthesis, W.H. Freeman Co., San Francisco, Calif. (1969); Merrifield, J. Am. Chem. Soc., 85:2149-2154 (1963)]. In vitro protein synthesis may be performed using manual techniques or by automation. Automated synthesis may be accomplished, for instance, using an Applied Biosystems Peptide Synthesizer (Foster City, Calif.) using manufacturer's instructions. Various portions of the antibody and/or binding polypeptide may be chemically synthesized separately and combined using chemical or enzymatic methods to produce the desired antibody and/or binding polypeptide.

Forms of the antibody and/or binding polypeptide may be recovered from culture medium or from host cell lysates. If membrane-bound, it can be released from the membrane using a suitable detergent solution (e.g., Triton-X 100) or by enzymatic cleavage. Cells employed in expression of antibody and/or binding polypeptide can be disrupted by various physical or chemical means, such as freeze-thaw cycling, sonication, mechanical disruption, or cell lysing agents.

It may be desired to purify antibody and/or binding polypeptide from recombinant cell proteins or polypeptides. The following procedures are exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of the antibody and/or binding polypeptide. Various methods of protein purification may be employed and such methods are known in the art and described for example in Deutscher, Methods in Enzymology, 182 (1990); Scopes, Protein Purification: Principles and Practice, Springer-Verlag, New York (1982). The purification step(s) selected will depend, for example, on the nature of the production process used and the particular antibody and/or binding polypeptide produced.

When using recombinant techniques, the antibody and/or binding polypeptide can be produced intracellularly, in the periplasmic space, or directly secreted into the medium. If the antibody and/or binding polypeptide is produced intracellularly, as a first step, the particulate debris, either host cells or lysed fragments, are removed, for example, by centrifugation or ultrafiltration. Carter et al., Bio/Technology 10:163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody and/or binding polypeptide is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody and/or binding polypeptide composition prepared from the cells can be purified using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the antibody. Protein A can be used to purify antibodies that are based on human γ1, γ2 or γ4 heavy chains (Lindmark et al., J. Immunol. Meth. 62:1-13 (1983)). Protein G is recommended for all mouse isotypes and for human γ3 (Guss et al., EMBO J. 5:15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody and/or binding polypeptide to be recovered.

Following any preliminary purification step(s), the mixture comprising the antibody and/or binding polypeptide of interest and contaminants may be subjected to low pH hydrophobic interaction chromatography using an elution buffer at a pH between about 2.5-4.5, preferably performed at low salt concentrations (e.g., from about 0-0.25M salt).

### V. Methods of Screening and/or Identifying FGFR3Antagonists with Desired Function

Techniques for generating FGFR3 antagonists such as antibodies, binding polypeptides, and/or small molecules have been described above. Additional FGFR3 antagonists such as anti-FGFR3 antibodies, binding polypeptides, and/or binding small molecules provided herein may be identified, screened for, or characterized for their physical/chemical properties and/or biological activities by various assays known in the art.

To select for a FGFR3 antagonists which induces cancer cell death, loss of membrane integrity as indicated by, e.g., propidium iodide (PI), trypan blue or 7AAD uptake may be assessed relative to a reference. A PI uptake assay can be performed in the absence of complement and immune effector cells. FGFR3-expressing tumor cells are incubated with medium alone or medium containing the appropriate a FGFR3 antagonist. The cells are incubated for a 3-day time period. Following each treatment, cells are washed and aliquoted into 35 mm strainer-capped 12×75 tubes (1 ml per tube, 3 tubes per treatment group) for removal of cell clumps. Tubes then receive PI (10 μg/ml). Samples may be analyzed using a FACSCAN® flow cytometer and FACSCONVERT® CellQuest software (Becton Dickinson). Those FGFR3 antagonists that induce statistically significant levels of cell death as determined by PI uptake may be selected as cell death-inducing antibodies, binding polypeptides or binding small molecules.

To screen for FGFR3 antagonists which bind to an epitope on or interact with a polypeptide bound by an antibody of interest, a routine cross-blocking assay such as that described in Antibodies, A Laboratory Manual, Cold Spring Harbor Laboratory, Ed Harlow and David Lane (1988), can be performed. This assay can be used to determine if a candidate FGFR3 antagonist binds the same site or epitope as a known antibody. Alternatively, or additionally, epitope mapping can be performed by methods known in the art. For example, the antibody and/or binding polypeptide sequence can be mutagenized such as by alanine scanning, to identify contact residues. The mutant antibody is initially tested for binding with polyclonal antibody and/or binding polypeptide to ensure proper folding. In a different method, peptides corresponding to different regions of a polypeptide can be used in competition assays with the candidate antibodies and/or polypeptides or with a candidate antibody and/or binding polypeptide and an antibody with a characterized or known epitope.

In some embodiments of any of the methods of screening and/or identifying, the FGFR3candidate antagonist is an antibody, binding polypeptide, binding small molecule, or polynucleotide. In some embodiments, the FGFR3 candidate antagonist is an antibody. In some embodiments, the FGFR3 antagonist is a small molecule.

In one embodiment, a FGFR3 antagonist is tested for its antigen binding activity, e.g., by known methods such as ELISA, Western blot, etc.

### VI. Pharmaceutical Formulations

Pharmaceutical formulations of a FGFR3 antagonist as described herein are prepared by mixing such antibody having the desired degree of purity with one or more optional pharmaceutically acceptable carriers (Remington's Pharmaceutical Sciences 16th edition, Osol, A. Ed. (1980)), in the form of lyophilized formulations or aqueous solutions. In some embodiments, the FGFR3 antagonist is a binding small molecule, an antibody, binding polypeptide, and/or polynucleotide. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one embodiment, a sHASEGP is combined with one or more additional glycosaminoglycanases such as chondroitinases.

Exemplary lyophilized formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredients as necessary for the particular indication being treated, preferably those with complementary activities that do not adversely affect each other. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the FGFR3 antagonist, which matrices are in the form of shaped articles, e.g., films, or microcapsules.

The formulations to be used for in vivo administration are generally sterile. Sterility may be readily accomplished, e.g., by filtration through sterile filtration membranes.

### VII. Articles of Manufacture

In another embodiment, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is a FGFR3 antagonist described herein. The label or package insert indicates that the composition is used for treating the condition of choice. Moreover, the article of manufacture may comprise (a) a first container with a composition contained therein, wherein the composition comprises an FGFR3 antagonist; and (b) a second container with a composition contained therein, wherein the composition comprises a further cytotoxic or otherwise therapeutic agent.

In some embodiments, the article of manufacture comprises a container, a label on said container, and a composition contained within said container; wherein the composition includes one or more reagents (e.g., primary antibodies (e.g., B-9 Santa Cruz Biotechnology antibody) that bind to one or more biomarkers or probes and/or primers to one or more of the biomarkers described herein), the label on the container indicating that the composition can be used to evaluate the presence of one or more biomarkers in a sample, and instructions for using the reagents for evaluating the presence of one or more biomarkers in a sample. The article of manufacture can further comprise a set of instructions and materials for preparing the sample and utilizing the reagents. In some embodiments, the article of manufacture may include reagents such as both a primary and secondary antibody, wherein the secondary antibody is conjugated to a label, e.g., an enzymatic label. In some embodiments, the article of manufacture one or more probes and/or primers to one or more of the biomarkers described herein. In some embodiments of any of the articles of manufacture, the one or more biomarkers is FGFR3.

In some embodiments of any of the article of manufacture, the FGFR3 antagonist is an antibody, binding polypeptide, binding small molecule, or polynucleotide. In some embodiments, the FGFR3 antagonist is a small molecule. In some embodiments, the FGFR3 antagonist is an antibody. In some embodiments, the antibody is a monoclonal antibody. In some embodiments, the antibody is a human, humanized, or chimeric antibody. In some embodiments, the antibody is an antibody fragment and the antibody fragment binds FGFR3.

The article of manufacture in this embodiment may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Other optional components in the article of manufacture include one or more buffers (e.g., block buffer, wash buffer, substrate buffer, etc.), other reagents such as substrate (e.g., chromogen) which is chemically altered by an enzymatic label, epitope retrieval solution, control samples (positive and/or negative controls), control slide(s) etc.

It is understood that any of the above articles of manufacture may include an immunoconjugate described herein in place of or in addition to a FGFR3 antagonist.

### EXAMPLES

The following are examples of methods and compositions. It is understood that various other embodiments may be practiced, given the general description provided above.

### Materials and Methods for Examples

Samples:

Either a formalin-fixed paraffin-embedded tumor specimens or unstained paraffin slides of a tumor sample or cancer cell line was analyzed.

Immunohistochemistry (IHC):

Formalin-fixed, paraffin-embedded tissue sections were deparaffinized prior to antigen retrieval, blocking and incubation with primary anti-FGFR3 antibodies. Following incubation with secondary antibody and enzymatic color development, sections were counterstained and dehydrated in series of alcohols and xylenes before coverslipping.

The following protocol was used for IHC. The Ventana Benchmark XT system was used to perform FGFR3 IHC staining using the following reagents and materials:

Primary antibody: anti-FGFR3 (B-9) Rabbit Monoclonal Primary Antibody (sc-13121)

Specimen Type Formalin-fixed paraffin embedded (FFPE) samples and control cell pellets of varying staining intensities

Procedure Species Human

Instrument: BenchMark XT

Epitope Recovery Conditions: Cell Conditioning, standard1 (CC1, Ventana, cat #950-124)

Primary Antibody Conditions: 1/200, diluent 951191 μg/ml/60 minutes at 37 C

Diluent: Ventana antibody dilution buffer (Tris HCl buffer, cat#95119)

Naive Antibody for negative control: Naive Mouse IgG at 3 μg/ml (Ventana Confirm negative control IgG)

Detection: Ultraview Universal DAB Detection kit (Benchmark Reagent, polymer system, Ventana cat #760-500) and amplification kit used according to manufacturer's instructions.

Counterstain: Ventana Hematoxylin II (cat #790-2208)/with Bluing reagent (Cat #760-2037) (8 minutes and 4 minutes, respectively)

The Benchmark XT Protocol was as follows:

1. paraffin (Selected)

2. Deparaffinization (Selected)

3. Cell Conditioning (Selected)

4. Conditioner #1 (Selected)

5. Standard CC1 (Selected)

6. Ab Incubation Temperatures (Selected)

7. 37C Ab Inc. (Selected)

8. Titration (Selected)

9. Hand Apply (Primary Antibody), and Incubate for (60 minutes)

10. Countstain (Selected)

11. Apply One Drop of (Hematoxylin II) (Countstain), Apply Coverslip, and Incubate for (8 minutes)

12. Post Counterstain (Selected)

13. Apply One Drop of (BLUING REAGENT) (Post Countstain), Apply Coverslip, and

Incubate for (4 minutes)

14. Wash slides in soap water to remove oil

15. Rinse slides with water

16. Dehydrate slides through 95% Ethanol, 100% Ethanol to xylene (Leica autostainer program #9)

17. Cover slip.

### Example 1

### Scoring FGFR3Expression by IHC

In urothelial carcinoma, neoplastic cells labeled with the FGFR-3 IHC assay were evaluated for percent positivity and intensity of the DAB signal. The immunohistochemical staining in urothelial carcinoma followed a membranous and/or cytoplasmic pattern. Irrespective of subcellular localization, the signal was classified as strong, moderate, weak, or negative.

Strong signal intensity was characterized by golden to dark brown, often granular, cytoplasmic and/or membrane staining that was detectable using 4× and 10× objectives. Moderate signal intensity was characterized by light brown to tan cytoplasmic and/or membrane staining detectable using 10× and 20× objectives. The moderate signal lacked the richness of brown color seen in cells with strong staining intensity; membranes were also thinner and overall staining was less granular. Weak signal intensity was characterized by pale tan to gray cytoplasmic and/or membrane staining just above the intensity of background that necessitated use of 20× and even 40× objectives in some cases. The weak signal lacked the brown tint seen in moderate staining intensity; membranes were very thin and delicate, and therefore not detectable at lower magnifications. Negative signal intensity was characterized by an absence of any detectable signal or a signal that was characterized as pale gray or grayish-blue and without evidence of membrane enhancement.

The signal was distributed homogeneously, having a uniform level of intensity throughout the neoplastic portions of the tumor or distributed heterogeneously having more than one intensity level. The relative percentages of signal intensities were visually estimated and used to generate a diagnostic score. In primary urothelial carcinoma samples, non-neoplastic urothelium exhibited variable staining ranging from negative to moderate membranous and/or cytoplasmic signal. A graphic of representative FGFR-3-labeled non-neoplastic urothelium is provided herein. An isotype negative control was used to evaluate the presence of background in test samples.

Staining required one serial tissue section for H&E, a second serial tissue section for anti-FGFR-3, and a third serial tissue section for the isotype negative control antibody. The anti-FGFR-3 OPM-2, KMS11, and RPMI8226 cell line control slides were used as run controls and a reference for assay specificity. A positive control tissue fixed and processed in the same manner as the individual specimens was run as a positive control for each set of test conditions and with every anti-FGFR-3 staining run performed. Control tissue were prepared from fresh autopsy/biopsy/surgical specimens and fixed as soon as possible in a manner identical to test tissue.

If the sample was inadequate based on the H&E evaluation, due to either absence of tumor or to presence of <50 viable tumor cells, a new sample will be requested. If the OPM-2, KMS11, and RPMI8226 cell line control slides were not acceptable, staining was repeated if sections were available. If the isotype negative control was not acceptable or the anti-FGFR-3 was not evaluable, then staining also was repeated. If the positive control tissue did not show positive staining of cytoplasmic and/or membrane staining of neoplastic cells as expected, the positive control was not acceptable and staining of the individual specimen together with the adequate controls were repeated. Unevaluable anti-FGFR-3 indicated that determination of reactivity was not possible due to necrosis, absence of tissue/tumor, artifacts of staining or fixation, or edge artifacts. If controls were acceptable and the anti-FGFR-3 was evaluable, the slide was scored by a trained pathologist as described in the Scoring Criteria section below.

Scoring Criteria

Following the evaluation of FGFR-3 IHC, a Clinical Score was assigned, and a Clinical Diagnosis (Dx) was determined. As illustrated in Table 2, a Clinical Diagnosis (Dx) of Negative was assigned to cases with Clinical Scores of 0. Cases with Clinical Scores of 1+, 2+ or 3+ were assigned a Positive Clinical Diagnosis. Clinical interpretation of urothelial carcinoma cases stained with anti-FGFR-3 (B-9) Mouse Monoclonal Antibody was based on the criteria noted in Table 2.

TABLE 2

| Clinical | Clinical | |
|---|---|---|
| Diagnosis | Score* | Staining Criteria |
| Negative | 0 | 1. Absent cytoplasmic and/or membrane staining |
| | | OR |
| | | 2. Cytoplasmic and/or membrane staining of ANY |
| | | intensity in <10% of cells |
| Positive | 1 | 1. ≧10% cytoplasmic and/or membrane staining |
| | | AND |
| | | 2. Weak cytoplasmic and/or membrane staining |
| | | with moderate and/or strong staining being <10% |
| | | of positively stained cells |
| | 2 | 1. ≧10% cytoplasmic and/or membrane staining |
| | | AND |
| | | 2. Moderate cytoplasmic and/or membrane staining |
| | | in ≧10% of cells, with strong staining being <10% |
| | | of positively stained cells; weak staining may or |
| | | may not be present |
| | 3 | 1. ≧10% cytoplasmic and/or membrane staining |
| | | AND |
| | | 2. Strong cytoplasmic and/or membrane staining |
| | | in ≧10% of positively staining cells; weak and |
| | | moderate staining may or may not be present |

Note:
If >=10% of tumor cells expressed FGFR-3, the clinical diagnosis was positive

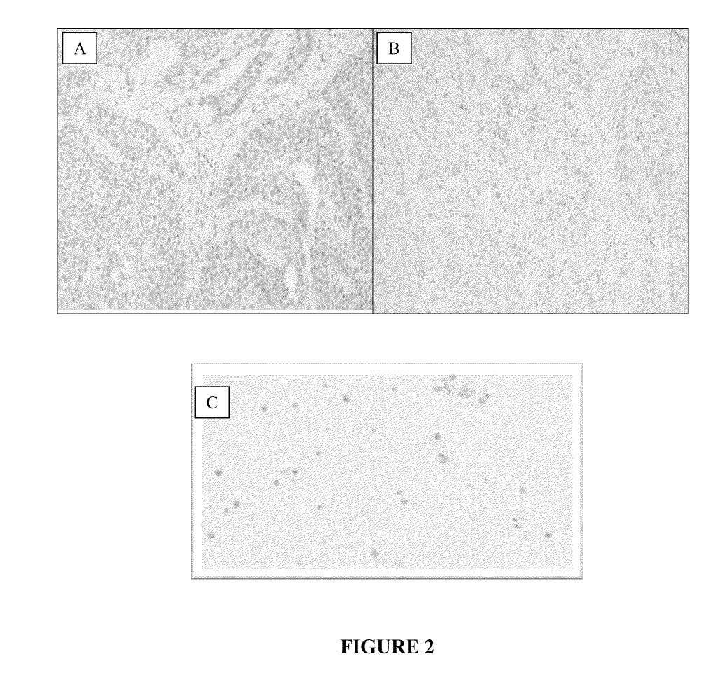

Evaluable slides stained with anti-FGFR-3 (B-9) were evaluated using the approach noted in the FIG. 1. Examples of Negative Cases (Clinical Score=0) are shown in FIG. 2A-B. Negative staining intensity was characterized by an absence of any detectable signal or a signal that was characterized as pale gray to blue (rather than brown or tan) and absence of membrane enhancement. The case was negative (Clinical Score=0) if <10% of neoplastic cells were positive (or >90% were negative) for the immunostain.

The H1155 Cell Line Control Slide represented a negative control with a Clinical Score of 0 (no or equivocal staining in tumor cells or <10% tumor cells with membrane and/or cytoplasmic staining of any intensity) as shown in FIG. 2C.

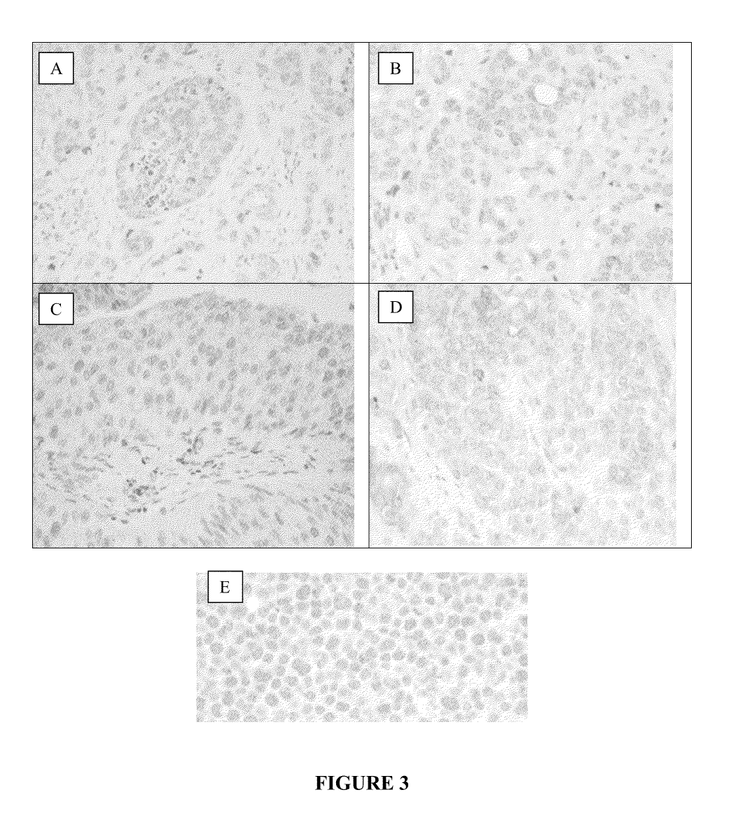

Anti-FGFR-3 Mouse Monoclonal Antibody stained cases with clinical score of 1 as shown in FIGS. 3A-D. Weak staining intensity was characterized by pale tan to gray cytoplasm and/or very delicate membrane enhancement (panels C and D). The weak signal lacked the brown tint seen in moderate staining intensity and membranes were thinner and not easily detectable at low magnification. The case was positive (Clinical Score=1) if >10% of neoplastic cells were of weak intensity AND those with MODERATE and STRONG intensity account for <10% of tumor cells.

The RPMI8226 Cell Line Control as shown in FIG. 3E represented a positive control with clinical score 1 in which >10% of tumor cells demonstrated weak cytoplasmic staining AND moderate and strong staining represents <10% of tumor cells. Cell Line RPM18226 had WEAK cytoplasmic or incomplete and delicate membrane staining (weak staining intensity). This cell line control did not demonstrate membranous staining.

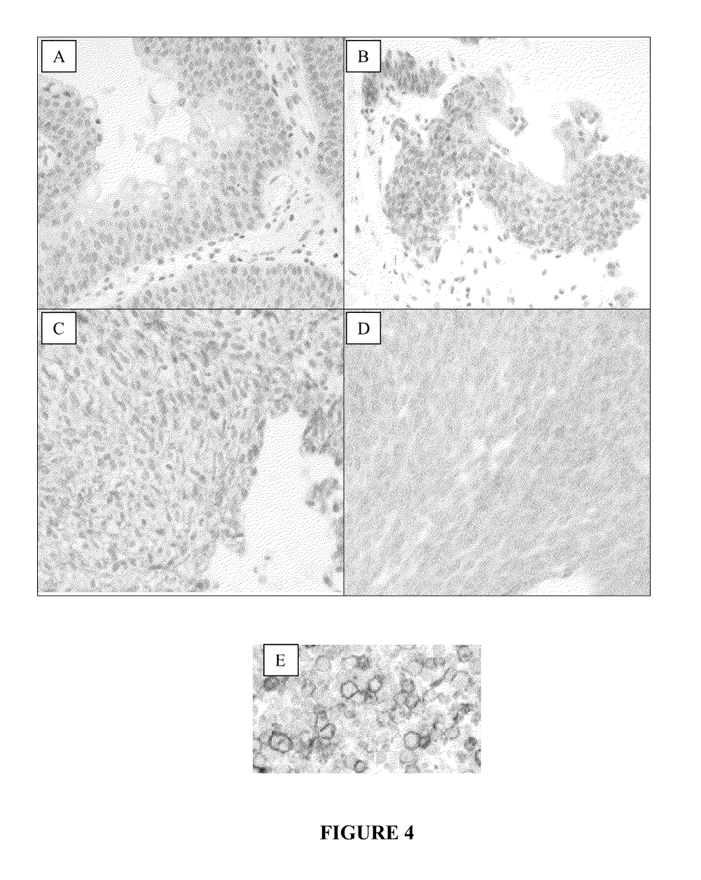

Anti-FGFR-3 Mouse Monoclonal Antibody stained cases with clinical score of 2 as shown in FIG. 4A-D. Moderate staining intensity was characterized by lighter brown to tan cytoplasm and/or mildly thickened membranes that were detectable at low to medium magnification. Moderate staining intensity lacked the rich brown color seen in strong staining intensity, and the membranes were less granular and thinner (Panel C). The case was positive (Clinical Score=2) if >10% of neoplastic cells were moderate for the immunostain AND <10% of tumor cells demonstrated STRONG staining.

The OPM2 Cell Line Control as shown in FIG. 4E represented a positive control with clinical score 2, characterized by circumferential membrane staining in ≦10% of cells; strong staining was seen in <10% of cells and weak cytoplasmic staining was seen in the majority of tumor cells. Cell Line OPM2 had MODERATE cytoplasmic and/or membrane staining (moderate staining intensity).

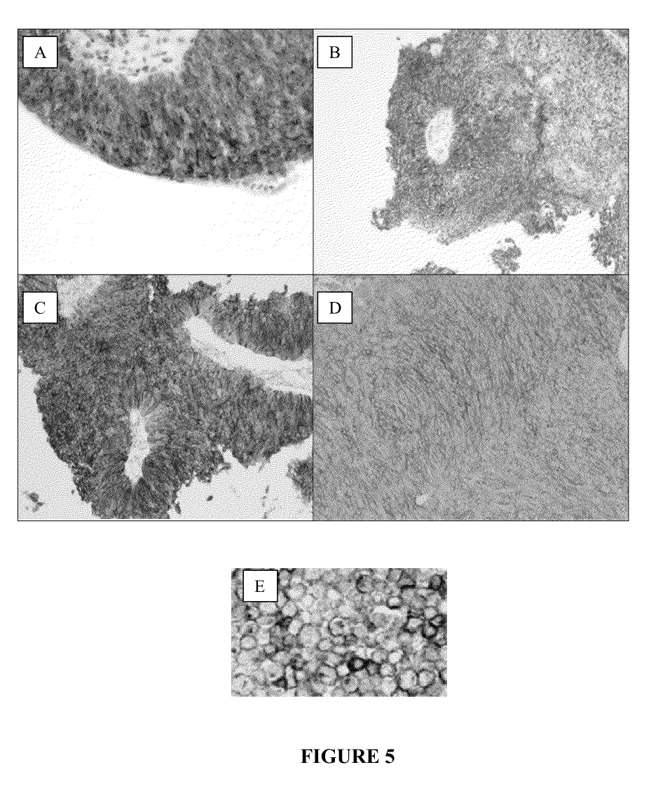

Anti-FGFR-3 Mouse Monoclonal Antibody stained cases with clinical score of 3 as shown in FIG. 5A-D. Strong staining intensity was characterized by golden to dark brown, often finely to coarsely granular cytoplasm and/or granular, golden brown to dark brown membranes of similar intensity that were usually detectable at low power. The case was positive (Clinical Score=3) if ≧10% of neoplastic cells were strong for the immunostain. Note that up to 90% of tumor cells with moderate and weak intensity may also be present.

The KMS11 Cell Line Control as shown in FIG. 5E represented a positive control with clinical score 3 characterized by thicker and darker, granular circumferential membranes in >10% of cells. Note that there were intermixed cells demonstrating moderate membrane staining. Cell Line KMS11 had STRONG cytoplasmic and/or circumferential and thickened membrane staining (strong staining intensity).

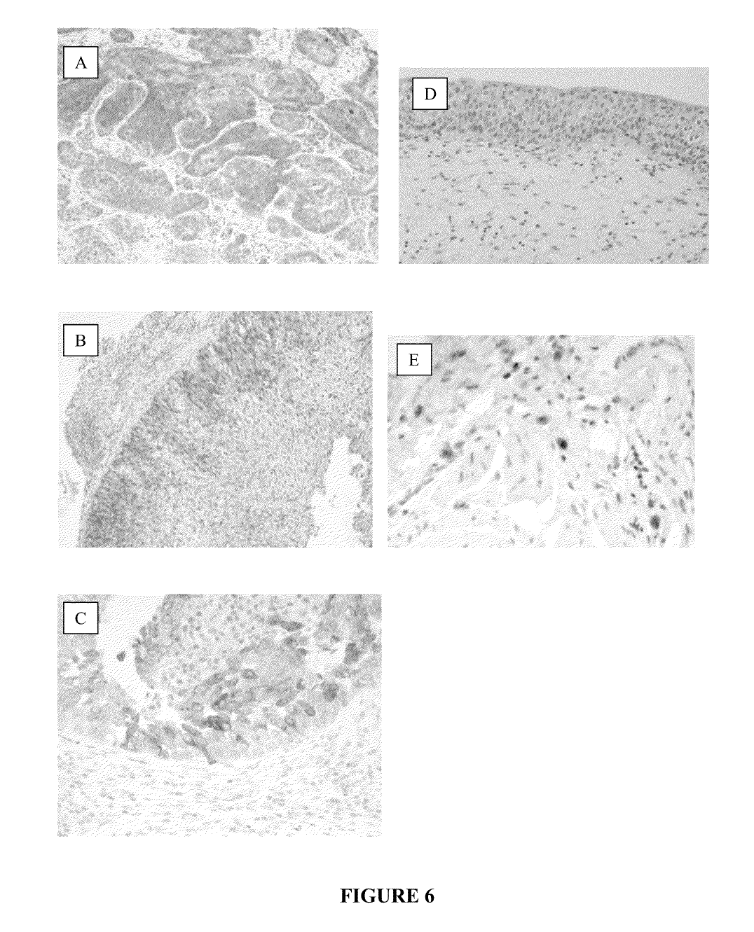

There was heterogeneity in urothelial carcinoma specimens as shown in FIG. 6A-C. In FIG. 6A, cytoplasmic staining ranging from weak to strong intensity could be seen in the field. Moderate and strong intensity membrane staining also could be seen. The clinical score for the sample was assessed as a 3. In FIG. 6B, membrane and cytoplasmic staining of moderate to strong intensity. The clinical score for the sample was assessed as a 3. In FIG. 6C, the range of cytoplasmic staining from negative to strong with focal, strong membrane staining. The clinical score for the sample was assessed as a 3.

The staining patterns in benign urothelium and in normal (non-epithelial) elements were shown in FIG. 6 D-E. In primary urothelial carcinoma samples, non-neoplastic urothelium exhibited variable staining ranging from negative to moderate membranous signal. Examples of the dynamic range of FGFR-3-labeled non-neoplastic urothelium were provided in FIG. 6D. Additionally, moderate and strong staining in normal elements (intramuscular mast cells) were seen (FIG. 6E).

Control Slides used for the anti-FGFR-3 Mouse Monoclonal Antibody consisted of formalin-fixed, paraffin-embedded cultured cell lines as follows: OPM-2, KMS11, and RPMI8226. These slides were intended to be used as assayed, semi-quantitative quality control material in conjunction with the Mouse Monoclonal Primary Antibody for use in monitoring the performance of the immunohistochemical anti-FGFR-3 staining process on an automated slide stainer. Staining was interpreted by a qualified pathologist in conjunction with histological examination and relevant clinical information.

TABLE 3
Scoring Forms:
Scoring Sheet Completion Guidelines

General Instructions

| | |
|---|---|
| 1. | Use blue or black ink. Do not use pencil. |
| 2. | Print your name, sign, and date each scoring sheet at the bottom. |
| | For dates, use the format DD-MMM-YYYY. For example May |
| | 3, 2011 would be 03-MAY-2011. |
| 3. | Corrections should be made with a single line through the error. |
| | Initial and date each correction. |

Scoring Instructions

| | |
|---|---|
| 1. | If the H&E slide was not acceptable, mark the H&E section of |
| | the scoring sheet as “Not Acceptable” and add a comment to |
| | Comments section. Continue to the negative control slide. |
| 2. | If the negative control slide was not acceptable, mark the negative |
| | control section of the scoring sheet as “Not Acceptable” and add a |
| | comment to Comments section. Continue to the FGFR-3 slide. |
| 3. | If the FGFR-3 slide was not evaluable, the case will be marked as |
| | Not Evaluable. |
| 4. | See the Scoring Criteria and Decision Tree for more details. |

### Example 2

### Scoring of FGFR3 IHC in Urothelial Carcinoma Panel

Definiens software was used to evaluate expression intensity of FGFR3 IHC in a panel of 150 urothelial carcinoma cases. Slides were stained as described above were scanned using a Hamamatsu Nanozoomer Digital Slide scanner running Nanozoomer software, with a 20× objective and 8 bit camera. All slides were only scanned in the area where specimen tissue was present. All images were analyzed using Definiens Developer (Munich, AG), using the RGB (red, green and blue) spectra. Images were downsampled by 2%, and tissue area was selected by excluding bright areas of the slide that correspond to background. Within the tissue, areas of stain were identified by searching for regions that had a normalized [red/blue] intensity value greater than 0.99. The mean brightness (mean value of red, green and blue spectra) was computed within the area of stain for each slide, as well as for unstained tissue area. Definiens denotes pixel intensities from 0 (darkest) to 255 (brightest). To obtain “stain intensity” the value [mean brightness of unstained tissue−mean brightness of stained tissue] was computed, where a larger value indicates relatively darker staining.

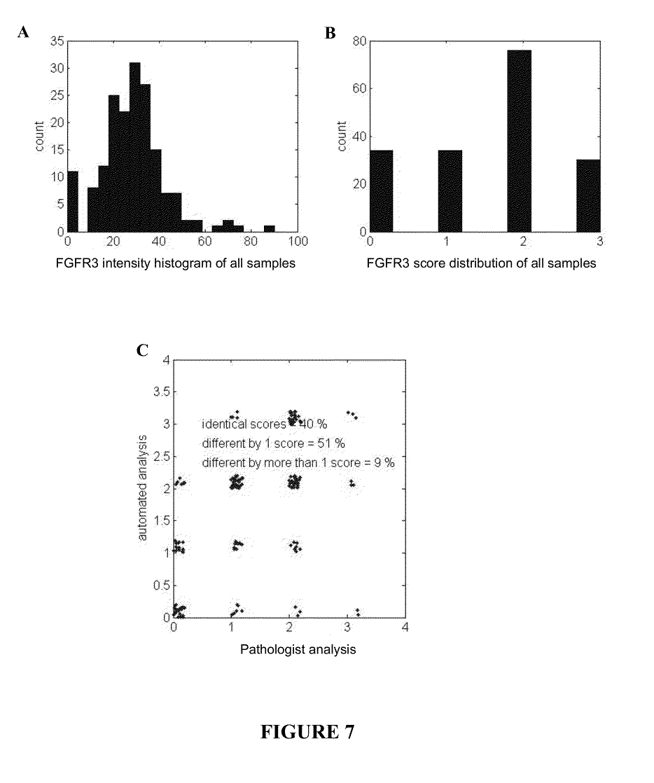

There was a range of expression as indicated by score distribution using the B9 anti-FGFR3 antibody (Santa Cruz Biotechnology sc-13121) (FIGS. 7A-B). The data demonstrated a clear distribution of staining intensity that correlated with pathologist scores (FIG. 7C).

### Example 3

### Treatment Using FGFR3Antibody R3MAb and Scoring of FGFR3 by IHC

FGFR3, a receptor tyrosine kinase, is implicated in cancer tumorigenesis. Anti-FGFR3 antibody R3Mab is a novel human monoclonal IgG antibody that suppressed FGFR3-medicated cell proliferation and exerts anti-tumor activity in xenograft models of urothelial cell carcinoma (UCC). See Clone 184.6.1° in FIG. 10. Preclinical data also supported the strategy of targeting FGFR3 in other solid tumors. This Phase I dose-escalation study evaluated the safety, pharmacokinetics (PK), and recommended Phase II dose (RP2D) of the anti-FGFR3 antibody R3MAb.

Using standard 3+3 design, patients with advanced solid malignancies refractory to standard therapy were treated with intravenous anti-FGFR3 antibody R3MAb in 5 dose-escalation cohorts (2-30 mg/kg, in 28-day cycles, with an additional loading dose on Cycle 1, Day 8). Cycle 1 comprised the dose-limiting toxicities (DLT) assessment window. Intra-patient dose escalation was allowed. Safety, PK, pharmacodynamics, and response were assessed.

Twenty-six (median age 63, range 21-77; 42% female) were dosed. One of 8 DLT-evaluable patients at 30 mg/kg experienced a DLT of Grade (G) 4 thrombocytopenia attributed to the anti-FGFR3 antibody R3Mab. This patient also had a confounding new concurrent medication, with rapid platelet recovery after discontinuation of both agents and administration of steroids. There were no other ≧G4 adverse events (AE). A maximum tolerated dose was not identified. G3 nausea was reported in 2 pts. AEs considered related to the anti-FGFR3 antibody R3MAb reported in ≧2 patients were fatigue (15%), nausea (12%), diarrhea, vomiting, mucosal inflammation, dyspnea, pruritus, and flushing (8% each). Two patients discontinued treatment due to an AE: one at 2 mg/kg due to G3 leukopenia attributed to the anti-FGFR3 antibody R3MAb, and another at 30 mg/kg due to G2 SAE sinus tachycardia not attributed to anti-FGFR3 antibody R3MAb. Preliminary PK analysis demonstrated a trend of dose proportional increase of exposure (area under the concentration-time curve) and maximal concentration from 2 to 8 mg/kg. Clearance was ˜0.35 L/day and central volume of distribution was ˜3.1 L, suggesting that the anti-FGFR3 antibody R3MAb has similar PK properties to other typical IgG monoclonal antibodies. Five of the 10 UCC patients had stable disease (SD) (4 SD, 1 non-CR/non-PD) as their best response. Four other patients had SD as their best response. Their tumor types included adenoid cystic carcinoma (n=2), and carcinoid tumor (n=2).

The RP2D of the anti-FGFR3 antibody R3MAb is 30 mg/kg. The anti-FGFR3 antibody R3MAb was well-tolerated with a favorable safety profile, and produced prolonged periods of disease stability in some patients.

### Example 4

### Scoring by IHC of FGFR3Samples from Individuals Treated with FGFR3Antibody R3MAb

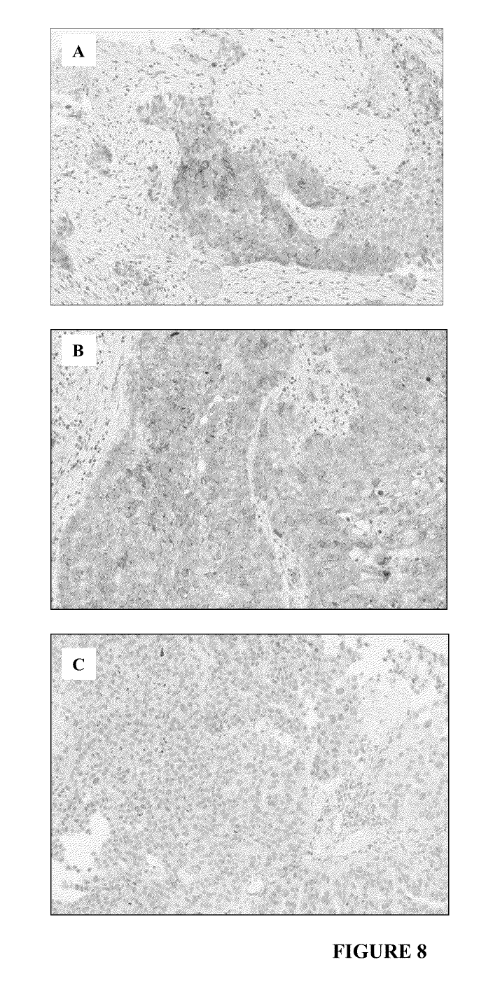

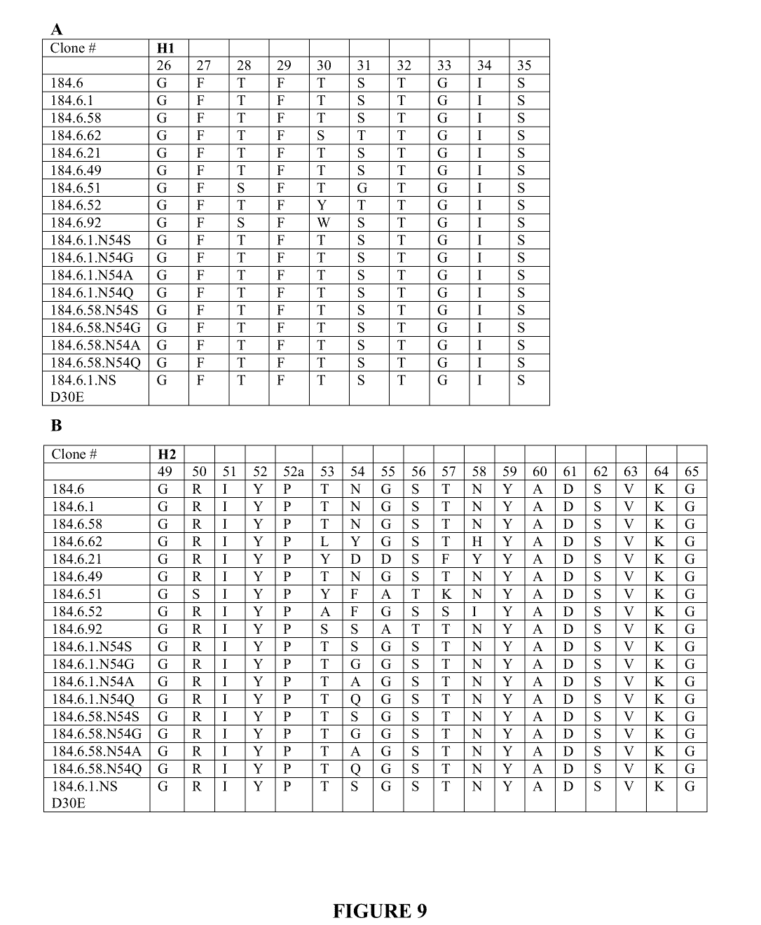

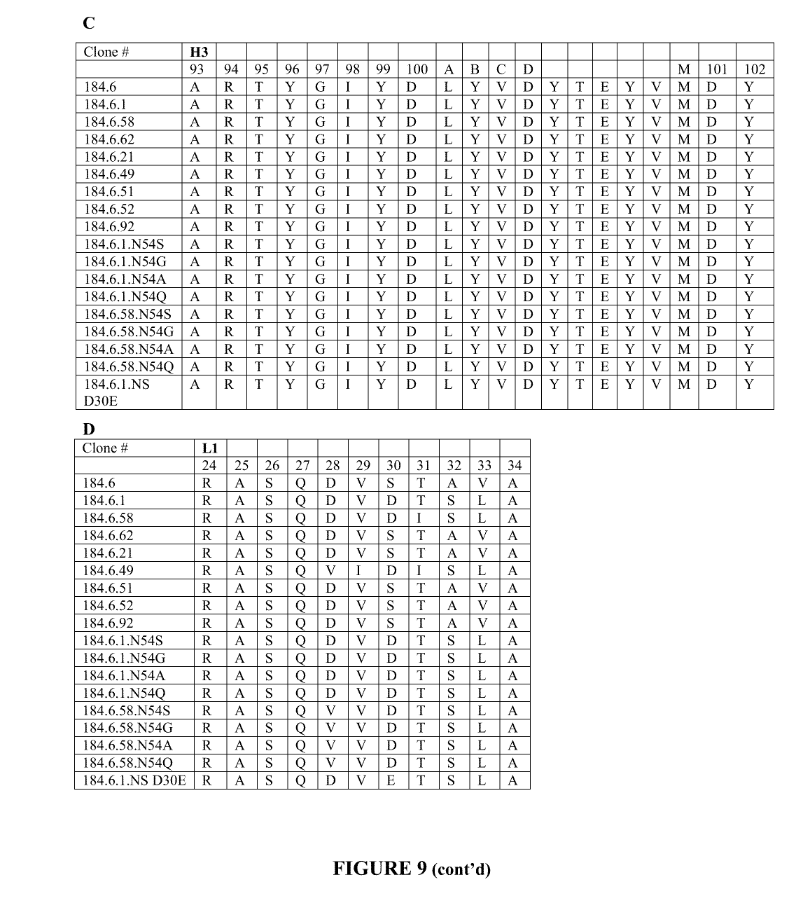

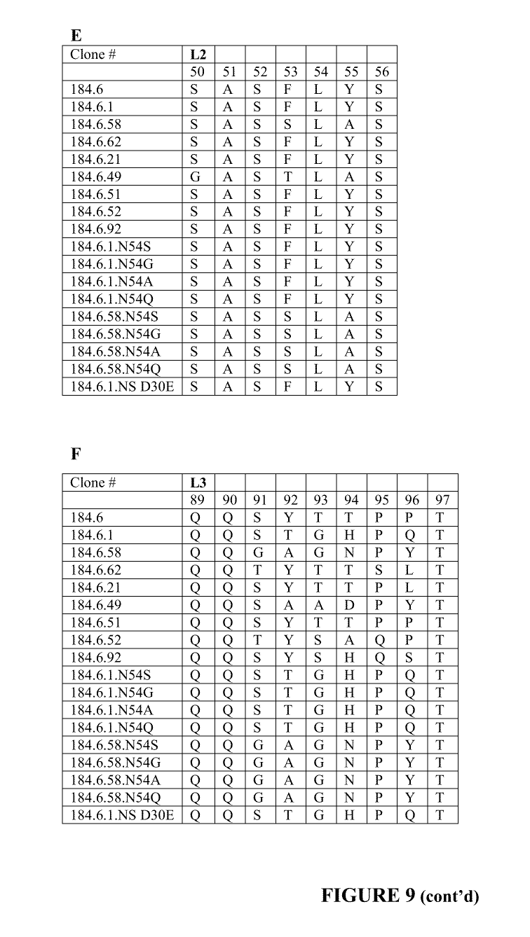

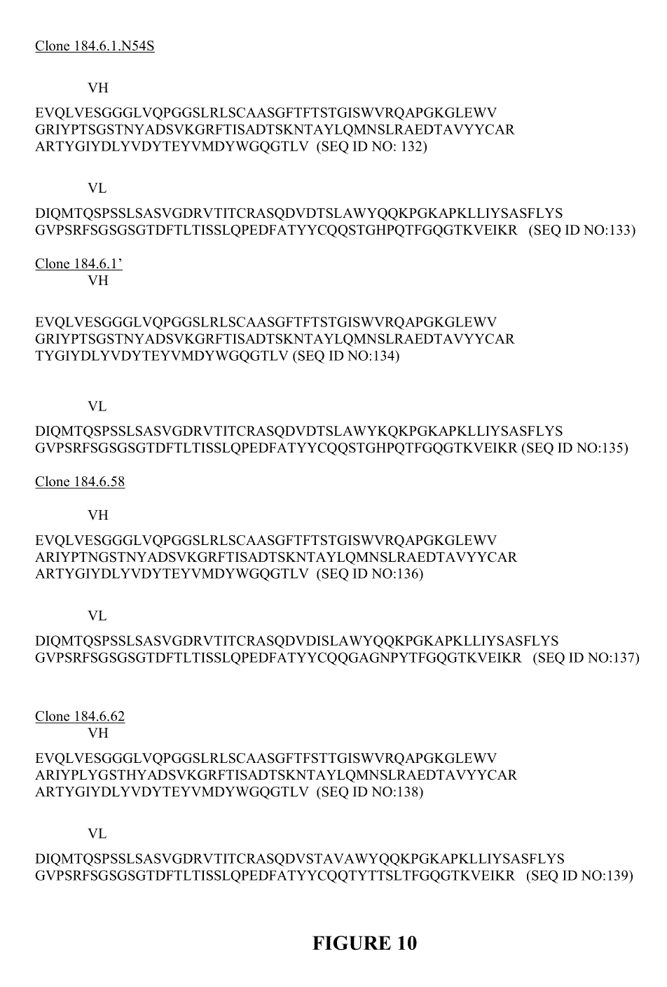

Pretreatment individual samples were analyzed from Phase I patients treated with the anti-FGFR3 antibody R3MAb. Results are shown below and exemplary examples are shown in FIG. 8. P-Progress. PD-Progressive Disease. SD-Stable Disease. No FGFR3 mutations were detected in patient samples.

TABLE 4

| | Best | Clinical | |
|---|---|---|---|
| Specimen | Response | Score | Comments |
| Patient 1 | P | 1 (positive) | basaloid colorectal carcinoma |
| Patient 2 | P | 2 (positive) | endometrioid ovarian carcinoma; |
| | | | there are areas in which the sub- |
| | | | apical cytoplasmic staining is |
| | | | more intense than the remaining |
| | | | of the cytosol; proteinaceous |
| | | | material demonstrates non-specific |
| | | | staining |
| Patient 3 | P | 0 (negative) | Thyroid; bony metastatic dis- |
| | | | ease (?) |
| Patient 4 | SD | 2 (positive) | UCC Patient; TUR specimen with |
| | SD to C10 | | presumed urothelial carcinoma |
| Patient 5 | P | 0 (negative) | STS |
| Patient 6 | P | 0 (negative) | HNSCC |
| Patient 7 | P | 2 (positive) | CRC; adenocarcinoma (frag- |
| | | | mented) |
| Patient 8 | SD | 2 (positive) | UCC Patient; dysplastic urothelium |
| | PD at C4 | | without invasive disease |
| Patient 9 | P | 3 (positive) | UCC Patient; TUR specimen with |
| | PD at C2 | | presumed urothelial carcinoma |
| Patient 10 | P | 2 (positive) | UCC Patient; urothelial carcinoma; |
| | PD at C2 | | extensive cautery artifact |
| Patient 11 | SD | | Carcinoid |
| Patient 12 | P | 0 (negative) | SCC |
| Patient 13 | SD | Not | UCC Patient |
| | C6−> | Evaluable | |
| Patient 14 | SD | 0 (negative) | UCC Patient |
| | C6−> | | |
| Patient 15 | SD | 0 (negative) | UCC Patient |
| | C6−> | | |
| Patient 16 | PD | 0 (negative) | UCC Patient |
| | PD at C2 | | |
| Patient 17 | PD | 0 (negative) | UCC Patient |
| | PD at C1 | | |
| Patient 18 | PD | 2 (positive) | UCC Patient |
| | PD at C1 | | |

### Example 5

### FGFR3 Knockdown Suppresses the Expression of Genes Cell Culture, siRNA Transfection and Reagents

The human bladder cancer cell lines SW780, BFTC-905 and Cal29 were obtained from ATCC. RT112 cells were purchased from German Collection of Microorganisms and Cell Cultures (DSMZ, Germany). RT112 cells stably expressing doxycycline-inducible shRNAs targeting FGFR3 or EGFP were previously described in (24). Bladder cancer cell line UMUC-14 was obtained from Dr. H.B. Grossman (Currently at University of Texas M. D. Anderson Cancer Center, TX) from the University of Michigan. Bladder cancer cell line TCC-97-7 was a gift from Dr. Margret Knowles of St. James's University Hospital (Leeds, United Kingdom). The cells were maintained with RPMI medium supplemented with 10% fetal bovine serum (FBS) (Sigma), 100 U/ml penicillin, 0.1 mg/ml streptomycin and L-glutamine under conditions of 5% CO₂ at 37° C.

All RNA interference experiments were carried out with ON-TARGETplus siRNAs (50 nM, Dharmacon, Lafayette, Colo.). Cells were transfected with Lipofectamine RNAiMax (Invitrogen, Carlsbad, Calif.), and cell proliferation or apoptosis were assessed 48 hr or 72 hr after transfection.

Gene Expression Array and Analyses

RT112 cells expressing doxycline-inducible shRNAs targeting FGFR3 or EGFP were grown in 10 cm plates in the presence or absence of doxycycline (1 μg/ml) for 48 hr. Total RNA from sub-confluent cell cultures was isolated using RNAeasy kit (Qiagen). RNA quality was verified by running samples on an Agilent Bioanalyzer 2100, and samples of sufficient quality were profiled on Affymetrix HGU133-Plus_{—}2.0 chips. Microarray studies were performed using triplicate RNA samples. Preparation of complementary RNA, array hybridizations, scanning, and subsequent array image data analysis were done following manufacturer's protocols. Expression summary values for all probe sets were calculated using the RMA algorithm as implemented in the affy package from Bioconductor. Statistical analyses of differentially expressed genes were performed using linear models and empirical Bayes moderated statistics as implemented in the limma package from Bioconductor. To obtain the biological processes that are over-represented by the differentially expressed genes, hypergeometric tests for association of Gene Ontology (GO) biological process categories and genes were performed using the GOstats and Category packages. Hierarchical clustering of the expression profile was performed using (1—Pearson's correlation) as the distance measure and Ward's minimum-variance method as the agglomeration method.

Preparation of BSA-Complexed Oleate and Palmitate

A 50 mM oleate or palmitate stock solution was prepared in 4 mM NaOH using the sodium salt of oleate or palmtate (Sigma-Aldrich). Fatty acid-free BSA (Sigma-Aldrich) was prepared in distilled H₂O at a final concentration of 4 mM. One volume of 50 mM stock of oleate or palmitate was combined with 1.5 volume of 4 mM BSA and heated to 55° C. for 1 hr to obtain a 20 mM stock solution of BSA-complexed oleate or palmitate at a fatty acid/BSA ratio of ˜8.3:1.

Cell Proliferation and Apoptosis Studies

For small interfering RNA experiments, at 72 hr after transfection, cells were processed for [Methyl-³H]thymidine incorporation. For doxycycline-inducible shRNA experiments, cells were treated with or without 1 μg/mL doxycyline for 72 hr before further incubation with [³H]thymidine for 16 hr. For SCD1 small molecule inhibitor experiment, cells were treated with indicated concentration of small molecule inhibitor in DMSO or DMSO alone for 48 hr. Cell viability was assessed with CellTiter-Glo (Promega). Values are presented as mean+/−SD of quadruplets.

Statistics

Pooled data were expressed as mean+/−SEM. Unpaired Student's t tests (2-tailed) were used for comparison between two groups. A value of P <0.05 was considered statistically significant in all experiments.

### Results

Using doxycycline-inducible shRNA, knockdown of FGFR3 in bladder cancer cell line RT112 significantly attenuated tumor growth in vitro and in vivo as previously shown in Qing et al. J. Clin. Invest. 119 (5):1216-1229 (2009). To identify potential FGFR3-downstream targets, the transcriptional profile of RT112-derived cell lines that express either the control shRNA or three independent FGFR3 shRNAs was compared. The use of three RT112-derived cell lines expressing different FGFR3 shRNAs provided a control for non-specific difference in these independently established cell lines. All cell lines were treated with or without doxycycline for 48 hours to deplete FGFR3 protein prior to the isolation of mRNA for microarray analysis. Genes that were differentially expressed (false discovery rate <0.1, fold change >2) upon doxycycline induction in all three FGFR3 shRNA cell lines but not in the control shRNA cells were considered potential FGFR3-regulated genes. Among the 19,701 genes represented on the array, 313 genes showed consistent differential expression in response to FGFR3 knockdown, with 196 upregulated and 117 downregulated. Results are shown in Table 5.

TABLE 5

| | | shRNA2 | shRNA4 | shRN6 |
|---|---|---|---|---|
| | | fold | fold | fold |
| | | change | change | change |
| Symbol | Name | (Log2) | (Log2) | (Log2) |
| FABP4 | fatty acid binding protein 4, adipocyte | −5.14 | −2.44 | −8.46 |
| PLAT | plasminogen activator, tissue | −2.95 | −3.84 | −3.42 |
| DUSP6 | dual specificity phosphatase 6 | −2.74 | −3 | −3.68 |
| FGFBP1 | fibroblast growth factor binding protein 1 | −3.54 | −2.26 | −5.2 |
| SCNN1B | sodium channel, nonvoltage-gated 1, beta | 3.02 | 3.62 | 3.27 |
| TRIM22 | tripartite motif containing 22 | 5.51 | 6.17 | 12 |
| UPK1A | uroplakin 1A | 3.15 | 2.84 | 4.2 |
| ID2 | inhibitor of DNA binding 2, dominant negative | 3.37 | 3.65 | 3.97 |
| | helix-loop-helix protein | | | |
| LDLR | low density lipoprotein receptor | −2.42 | −2.53 | −3.84 |
| LOXL1 | lysyl oxidase-like 1 | −2.3 | −2.97 | −3.98 |
| IDI1 | isopentenyl-diphosphate delta isomerase 1 | −2.47 | −2.16 | −3.09 |
| SEPP1 | selenoprotein P, plasma, 1 | 3.47 | 6.13 | 6.52 |
| FDFT1 | farnesyl-diphosphate farnesyltransferase 1 | −2.58 | −1.91 | −3.49 |
| CCDC85A | coiled-coil domain containing 85A | 4.74 | 4.36 | 8.99 |
| MUC15 | mucin 15, cell surface associated | 2.68 | 3.04 | 4.97 |
| SC4MOL | sterol-C4-methyl oxidase-like | −3.18 | −2.01 | −3.92 |
| CRISP3 | cysteine-rich secretory protein 3 | 2.49 | 4.26 | 4.88 |
| S100A2 | S100 calcium binding protein A2 | −1.82 | −3.06 | −2.63 |
| ERP27 | endoplasmic reticulum protein 27 | 2.75 | 2.22 | 4.25 |
| FRAS1 | Fraser syndrome 1 | 5.97 | 4.54 | 4.66 |
| PCSK9 | proprotein convertase subtilisin/kexin type 9 | −3.3 | −3.11 | −5.21 |
| SQLE | squalene epoxidase | −3.52 | −2.47 | −5.31 |
| CYP4B1 | cytochrome P450, family 4, subfamily B, | 1.96 | 2.2 | 3.56 |
| | polypeptide 1 | | | |
| IGHA1 | immunoglobulin heavy constant alpha 1 | 2.29 | 4.8 | 3.01 |
| MMP1 | matrix metallopeptidase 1 (interstitial collagenase) | −7.45 | −12 | −21.4 |
| F2R | coagulation factor II (thrombin) receptor | −2.31 | −4.15 | −2.36 |
| TSPAN12 | tetraspanin 12 | −2.55 | −2.68 | −3.15 |
| ABP1 | amiloride binding protein 1 (amine oxidase | 2 | 2.11 | 3.65 |
| | (copper-containing)) | | | |
| COL4A4 | collagen, type IV, alpha 4 | 2.44 | 2.95 | 6.42 |
| INSIG1 | insulin induced gene 1 | −3.04 | −2.23 | −4.32 |
| SLCO4A1 | solute carrier organic anion transporter family, | −1.84 | −2.91 | −2.98 |
| | member 4A1 | | | |
| PDE8B | phosphodiesterase 8B | 3.57 | 3.77 | 3.9 |
| ATP1A4 | ATPase, Na+/K+ transporting, alpha 4 polypeptide | 2.27 | 3.83 | 3.56 |
| CLDN8 | claudin 8 | 2.97 | 3.45 | 4.55 |
| NT5E | 5′-nucleotidase, ecto (CD73) | −2.79 | −3.48 | −3.42 |
| TNS1 | tensin 1 | 1.88 | 2.37 | 4.35 |
| VSIG2 | V-set and immunoglobulin domain containing 2 | 1.77 | 2.7 | 2.53 |
| PHLDA1 | pleckstrin homology-like domain, family A, | −2.37 | −3.12 | −2.54 |
| | member 1 | | | |
| SCNN1G | sodium channel, nonvoltage-gated 1, gamma | 2.54 | 2.26 | 3.33 |
| COL4A2 | collagen, type IV, alpha 2 | −1.72 | −2.58 | −2.03 |
| FGFR3 | fibroblast growth factor receptor 3 | −1.84 | −2.87 | −3.29 |
| HMGCS1 | 3-hydroxy-3-methylglutaryl-CoA synthase 1 | −3.09 | −1.82 | −3.26 |
| | (soluble) | | | |
| S100A9 | S100 calcium binding protein A9 | −2.07 | −1.72 | −2.6 |
| VTCN1 | V-set domain containing T cell activation inhibitor 1 | 2.27 | 3.16 | 3.36 |
| CCDC80 | coiled-coil domain containing 80 | 2.46 | 2.3 | 3.21 |
| SPATA17 | spermatogenesis associated 17 | 2.21 | 2.24 | 3.15 |
| MAN1A1 | mannosidase, alpha, class 1A, member 1 | 2.58 | 2.48 | 3.62 |
| SPOCK1 | sparc/osteonectin, cwcv and kazal-like domains | 1.97 | 2.04 | 2.48 |
| | proteoglycan (testican) 1 | | | |
| SULF2 | sulfatase 2 | −2.32 | −2.42 | −2.3 |
| ACAT2 | acetyl-CoA acetyltransferase 2 | −2.17 | −1.87 | −2.61 |
| MUC20 | mucin 20, cell surface associated | 1.68 | 2.07 | 2.85 |
| MMP10 | matrix metallopeptidase 10 (stromelysin 2) | −3.61 | −3.68 | −5.97 |
| TMC4 | transmembrane channel-like 4 | 1.67 | 2.33 | 2.52 |
| HMGCR | 3-hydroxy-3-methylglutaryl-CoA reductase | −2.13 | −1.6 | −2.5 |
| CDK14 | cyclin-dependent kinase 14 | 2.18 | 1.95 | 3.82 |
| FASN | fatty acid synthase | −2.03 | −1.65 | −3.26 |
| ATP6V1B1 | ATPase, H+ transporting, lysosomal 56/58 kDa, V1 | 2.12 | 2.32 | 2.78 |
| | subunit B1 | | | |
| DHRS2 | dehydrogenase/reductase (SDR family) member 2 | 2.13 | 2.1 | 2.5 |
| TNS3 | tensin 3 | 1.7 | 1.89 | 2.64 |
| ATP2B4 | ATPase, Ca++ transporting, plasma membrane 4 | 1.72 | 1.76 | 2.56 |
| PDZK1 | PDZ domain containing 1 | 2.52 | 2.13 | 4.02 |
| MYCL1 | v-myc myelocytomatosis viral oncogene homolog | 1.86 | 2.33 | 2.39 |
| | 1, lung carcinoma derived (avian) | | | |
| CYB5B | cytochrome b5 type B (outer mitochondrial | −2.03 | −2.24 | −2.23 |
| | membrane) | | | |
| KRT15 | keratin 15 | 1.64 | 1.84 | 2.44 |
| DAPL1 | death associated protein-like 1 | 1.73 | 3.55 | 2.98 |
| FAR2 | fatty acyl CoA reductase 2 | −2.37 | −2.59 | −2.79 |
| DHCR7 | 7-dehydrocholesterol reductase | −2.23 | −1.61 | −2.92 |
| ASPH | aspartate beta-hydroxylase | −1.84 | −1.75 | −2.41 |
| CFD | complement factor D (adipsin) | 2.18 | 3.32 | 2.38 |
| IFIT1 | interferon-induced protein with tetratricopeptide | 1.89 | 2.23 | 3.09 |
| | repeats 1 | | | |
| MR1 | major histocompatibility complex, class I-related | 2.29 | 2.43 | 3.17 |
| OLR1 | oxidized low density lipoprotein (lectin-like) | 1.68 | 1.78 | 3.2 |
| | receptor 1 | | | |
| C3orf58 | chromosome 3 open reading frame 58 | 1.53 | 1.63 | 2.37 |
| DHRS9 | dehydrogenase/reductase (SDR family) member 9 | −1.53 | −3.97 | −5.03 |
| IQGAP2 | IQ motif containing GTPase activating protein 2 | −2.77 | −1.61 | −3.32 |
| PPP1R3B | protein phosphatase 1, regulatory (inhibitor) | 1.67 | 2.47 | 2.48 |
| | subunit 3B | | | |
| HS3ST1 | heparan sulfate (glucosamine) 3-O-sulfotransferase 1 | −2.01 | −1.69 | −2.85 |
| C16orf54 | chromosome 16 open reading frame 54 | −2.24 | −5.57 | −1.96 |
| FGD3 | FYVE, RhoGEF and PH domain containing 3 | 1.51 | 1.73 | 2.03 |
| PIK3IP1 | phosphoinositide-3-kinase interacting protein 1 | 1.83 | 2.14 | 2.1 |
| LGALS8 | lectin, galactoside-binding, soluble, 8 | −2.08 | −1.87 | −2.17 |
| OPTN | optineurin | 1.72 | 1.8 | 2.18 |
| LAMB3 | laminin, beta 3 | 1.89 | 2.01 | 2.23 |
| SCD | stearoyl-CoA desaturase (delta-9-desaturase) | −3.01 | −3.76 | −5.04 |
| GKN1 | gastrokine 1 | 2.32 | 2.92 | 2.5 |
| MICB | MHC class I polypeptide-related sequence B | −2.68 | −2.7 | −2.94 |
| ID1 | inhibitor of DNA binding 1, dominant negative | 1.99 | 1.76 | 1.82 |
| | helix-loop-helix protein | | | |
| SPTLC3 | serine palmitoyltransferase, long chain base subunit 3 | 1.51 | 2 | 2.19 |
| ETV4 | ets variant 4 | −1.84 | −1.97 | −2.59 |
| ACSL3 | acyl-CoA synthetase long-chain family member 3 | −1.93 | −1.77 | −1.75 |
| SLC20A1 | solute carrier family 20 (phosphate transporter), | −1.82 | −1.69 | −2.22 |
| | member 1 | | | |
| TSC22D3 | TSC22 domain family, member 3 | 1.69 | 1.78 | 1.84 |
| DBP | D site of albumin promoter (albumin D-box) | 2.1 | 1.96 | 2.29 |
| | binding protein | | | |
| IGFBP5 | insulin-like growth factor binding protein 5 | 1.79 | 2.75 | 8.55 |
| CYP1B1 | cytochrome P450, family 1, subfamily B, | 2.26 | 2.81 | 3.09 |
| | polypeptide 1 | | | |
| CDC42EP3 | CDC42 effector protein (Rho GTPase binding) 3 | 1.65 | 1.7 | 2.72 |
| SLC35A1 | solute carrier family 35 (CMP-sialic acid | 1.53 | 1.69 | 2.31 |
| | transporter), member A1 | | | |
| ID3 | inhibitor of DNA binding 3, dominant negative | 2.03 | 2.08 | 2.17 |
| | helix-loop-helix protein | | | |
| ITGA2 | integrin, alpha 2 (CD49B, alpha 2 subunit of VLA- | −1.65 | −2.22 | −1.73 |
| | 2 receptor) | | | |
| FOXO6 | forkhead box O6 | 1.7 | 1.88 | 2.25 |
| NDRG1 | N-myc downstream regulated 1 | 1.91 | 1.74 | 1.95 |
| TBX3 | T-box 3 | 1.65 | 1.99 | 2.07 |
| SEZ6L2 | seizure related 6 homolog (mouse)-like 2 | 1.77 | 2.08 | 1.8 |
| WNT4 | wingless-type MMTV integration site family, | 2.02 | 2.13 | 2.4 |
| | member 4 | | | |
| HOXA5 | homeobox A5 | 1.65 | 2.1 | 2.18 |
| LRP8 | low density lipoprotein receptor-related protein 8, | −2.86 | −2.6 | −4.12 |
| | apolipoprotein e receptor | | | |
| PAICS | phosphoribosylaminoimidazole carboxylase, | −1.7 | −1.82 | −1.87 |
| | phosphoribosylaminoimidazole | | | |
| | succinocarboxamide synthetase | | | |
| C10orf54 | chromosome 10 open reading frame 54 | 1.66 | 1.75 | 2.35 |
| ELOVL5 | ELOVL family member 5, elongation of long chain | −2.18 | −1.72 | −2.13 |
| | fatty acids (FEN1/Elo2, SUR4/Elo3-like, yeast) | | | |
| CTNNAL1 | catenin (cadherin-associated protein), alpha-like 1 | −1.63 | −2.53 | −1.67 |
| SEMA3E | sema domain, immunoglobulin domain (Ig), short | 2.1 | 2.6 | 3.2 |
| | basic domain, secreted, (semaphorin) 3E | | | |
| PFKFB3 | 6-phosphofructo-2-kinase/fructose-2,6- | 1.79 | 1.93 | 2.55 |
| | biphosphatase 3 | | | |
| KITLG | KIT ligand | 1.69 | 1.78 | 2.18 |
| BCL11A | B-cell CLL/lymphoma 11A (zinc finger protein) | 1.56 | 1.93 | 2.49 |
| NEBL | nebulette | 1.86 | 1.93 | 2.58 |
| TIMP2 | TIMP metallopeptidase inhibitor 2 | 1.64 | 1.94 | 2.73 |
| STARD5 | StAR-related lipid transfer (START) domain | 1.63 | 1.9 | 2.06 |
| | containing 5 | | | |
| IL1RN | interleukin 1 receptor antagonist | 1.79 | 2.06 | 1.7 |
| PCDHB14 | protocadherin beta 14 | 1.96 | 3.22 | 2.87 |
| MVP | major vault protein | 1.54 | 2.18 | 1.74 |
| TMEM47 | transmembrane protein 47 | −2.29 | −2.63 | −2.57 |
| CHAC2 | ChaC, cation transport regulator homolog 2 (E. coli) | −2.82 | −2.02 | −2.66 |
| OLFML2A | olfactomedin-like 2A | 1.62 | 1.74 | 1.9 |
| GDA | guanine deaminase | −1.55 | −1.78 | −1.68 |
| MMD | monocyte to macrophage differentiation-associated | −2.04 | −2.26 | −1.51 |
| ALDH3B1 | aldehyde dehydrogenase 3 family, member B1 | 1.56 | 1.81 | 1.92 |
| NME1 | non-metastatic cells 1, protein (NM23A) expressed in | −2.02 | −1.51 | −2.18 |
| CLU | clusterin | 1.57 | 2.12 | 2.15 |
| APOBEC3G | apolipoprotein B mRNA editing enzyme, catalytic | −2.67 | −1.94 | −2.98 |
| | polypeptide-like 3G | | | |
| DDX39A | DEAD (Asp-Glu-Ala-Asp) box polypeptide 39A | −1.63 | −1.79 | −1.62 |
| | (SEQ ID NO: 182) | | | |
| HBEGF | heparin-binding EGF-like growth factor | −1.93 | −1.95 | −2.14 |
| PNP | purine nucleoside phosphorylase | −1.77 | −1.85 | −2.11 |
| FDPS | farnesyl diphosphate synthase | −1.85 | −1.77 | −2.06 |
| FAM171B | family with sequence similarity 171, member B | 1.6 | 2.87 | 3.2 |
| ERO1L | ERO1-like (S. cerevisiae) | −1.7 | −1.74 | −1.68 |
| ADORA2B | adenosine A2b receptor | −1.69 | −1.65 | −1.87 |
| CYP51A1 | cytochrome P450, family 51, subfamily A, | −2.15 | −2.28 | −3.29 |
| | polypeptide 1 | | | |
| TUBG1 | tubulin, gamma 1 | −1.6 | −1.88 | −1.62 |
| LSS | lanosterol synthase (2,3-oxidosqualene-lanosterol | −1.98 | −1.9 | −2.78 |
| | cyclase) | | | |
| STOX2 | storkhead box 2 | 1.99 | 2.47 | 3.45 |
| CTPS | CTP synthase | −1.9 | −1.61 | −2.06 |
| ABAT | 4-aminobutyrate aminotransferase | 1.61 | 2.34 | 3.78 |
| SEPW1 | selenoprotein W, 1 | 1.56 | 1.54 | 1.96 |
| GABRP | gamma-aminobutyric acid (GABA) A receptor, pi | 2.2 | 1.74 | 2.96 |
| TACC3 | transforming, acidic coiled-coil containing protein 3 | −1.52 | −2.15 | −2.01 |
| TCF7L1 | transcription factor 7-like 1 (T-cell specific, HMG- | 1.67 | 1.51 | 2.02 |
| | box) | | | |
| TFPI2 | tissue factor pathway inhibitor 2 | −1.68 | −2.29 | −1.72 |
| FYB | FYN binding protein | 3.11 | 2.54 | 2.57 |
| MATN2 | matrilin 2 | 1.8 | 1.69 | 2.5 |
| WNT10A | wingless-type MMTV integration site family, | 1.8 | 1.79 | 1.9 |
| | member 10A | | | |
| TFRC | transferrin receptor (p90, CD71) | −2.23 | −2.99 | −3.31 |
| RIMS2 | regulating synaptic membrane exocytosis 2 | 1.71 | 1.69 | 2.23 |
| PSMD14 | proteasome (prosome, macropain) 26S subunit, | −1.65 | −1.62 | −1.74 |
| | non-ATPase, 14 | | | |
| GRHL3 | grainyhead-like 3 (Drosophila) | 1.5 | 2.02 | 1.53 |
| ZFP36L1 | zinc finger protein 36, C3H type-like 1 | 1.75 | 2.05 | 1.79 |
| TSGA10 | testis specific, 10 | 1.96 | 2.08 | 3.23 |
| GART | phosphoribosylglycinamide formyltransferase, | −1.92 | −1.73 | −1.81 |
| | phosphoribosylglycinamide synthetase, | | | |
| | phosphoribosylaminoimidazole synthetase | | | |
| SLC45A3 | solute carrier family 45, member 3 | −1.84 | −1.71 | −2.58 |
| ATL1 | atlastin GTPase 1 | 1.79 | 2.2 | 2.03 |
| ANKDD1A | ankyrin repeat and death domain containing 1A | 1.67 | 1.65 | 2.57 |
| ACPL2 | acid phosphatase-like 2 | −1.6 | −1.65 | −1.56 |
| ITLN1 | intelectin 1 (galactofuranose binding) | 2.14 | 2.6 | 3.79 |
| C20orf114 | chromosome 20 open reading frame 114 | 1.64 | 1.75 | 2.25 |
| ARHGAP26 | Rho GTPase activating protein 26 | 1.54 | 1.73 | 1.8 |
| CYP24A1 | cytochrome P450, family 24, subfamily A, | −1.56 | −2.41 | −1.54 |
| | polypeptide 1 | | | |
| HIST1H2AC | histone cluster 1, H2ac | 2.02 | 1.79 | 1.83 |
| FAM49A | family with sequence similarity 49, member A | 1.71 | 1.7 | 2.18 |
| PLD1 | phospholipase D1, phosphatidylcholine-specific | 1.59 | 1.8 | 2.17 |
| TMPRSS2 | transmembrane protease, serine 2 | 1.89 | 2.38 | 2.07 |
| PP14571 | similar to hCG1777210 | 1.58 | 1.72 | 2.47 |
| MAFB | v-maf musculoaponeurotic fibrosarcoma oncogene | 1.73 | 3.57 | 2.07 |
| | homolog B (avian) | | | |
| SDR16C5 | short chain dehydrogenase/reductase family 16C, | −2.28 | −2.03 | −3.94 |
| | member 5 | | | |
| WDR4 | WD repeat domain 4 | −1.94 | −1.5 | −2.11 |
| TNIK | TRAF2 and NCK interacting kinase | −1.65 | −1.75 | −1.78 |
| FAM46A | family with sequence similarity 46, member A | 1.99 | 2.41 | 2.69 |
| FAM134B | family with sequence similarity 134, member B | 1.74 | 1.87 | 2.71 |
| SEMA5A | sema domain, seven thrombospondin repeats (type | 1.57 | 2.28 | 1.98 |
| | 1 and type 1-like), transmembrane domain (TM) | | | |
| | and short cytoplasmic domain, (semaphorin) 5A | | | |
| PRICKLE1 | prickle homolog 1 (Drosophila) | 2.02 | 2.24 | 2.76 |
| ID4 | inhibitor of DNA binding 4, dominant negative | 3.91 | 4.05 | 3.21 |
| | helix-loop-helix protein | | | |
| PPP2R2B | protein phosphatase 2, regulatory subunit B, beta | 1.53 | 1.65 | 2.24 |
| MGC16075 | hypothetical protein MGC16075 | 2.15 | 1.75 | 2.27 |
| ZNF404 | zinc finger protein 404 | 1.72 | 1.97 | 3.34 |
| IFI44 | interferon-induced protein 44 | 1.55 | 2.25 | 2.22 |
| SMPDL3A | sphingomyelin phosphodiesterase, acid-like 3A | 1.51 | 1.67 | 2.03 |
| JDP2 | Jun dimerization protein 2 | 1.8 | 1.95 | 3.13 |
| CD55 | CD55 molecule, decay accelerating factor for | 1.72 | 2.2 | 2.38 |
| | complement (Cromer blood group) | | | |
| ZIC2 | Zic family member 2 (odd-paired homolog, | −1.78 | −1.76 | −1.64 |
| | Drosophila) | | | |
| C6orf141 | chromosome 6 open reading frame 141 | −2.06 | −2.65 | −2.09 |
| CPAMD8 | C3 and PZP-like, alpha-2-macroglobulin domain | 1.73 | 1.71 | 1.78 |
| | containing 8 | | | |
| ME1 | malic enzyme 1, NADP(+)-dependent, cytosolic | −2.42 | −1.63 | −2.6 |
| GGT6 | gamma-glutamyltransferase 6 | 1.63 | 2.08 | 1.79 |
| C17orf103 | chromosome 17 open reading frame 103 | 1.56 | 1.64 | 2.05 |
| FAM84A | family with sequence similarity 84, member A | 1.58 | 1.79 | 2.13 |
| CLIC5 | chloride intracellular channel 5 | 1.61 | 1.66 | 2.75 |
| KAL1 | Kallmann syndrome 1 sequence | 1.65 | 1.52 | 2.36 |
| APCDD1 | adenomatosis polyposis coli down-regulated 1 | 1.96 | 2.19 | 2.91 |
| MT1F | metallothionein 1F | 1.54 | 1.95 | 1.76 |
| MPPED2 | metallophosphoesterase domain containing 2 | 1.67 | 1.72 | 2.72 |
| SYNPO | synaptopodin | 1.6 | 1.72 | 1.57 |
| TRIM16 | tripartite motif containing 16 | 1.64 | 2.01 | 1.98 |
| TSPAN8 | tetraspanin 8 | 1.97 | 1.78 | 1.81 |
| ARNT | aryl hydrocarbon receptor nuclear translocator | 1.57 | 1.98 | 2.15 |
| DAPK2 | death-associated protein kinase 2 | 2.12 | 1.89 | 2.08 |
| SH3BGRL | SH3 domain binding glutamic acid-rich protein like | 1.71 | 1.79 | 3.23 |
| PLK1 | polo-like kinase 1 | −1.51 | −1.89 | −1.52 |
| MBIP | MAP3K12 binding inhibitory protein 1 | 1.54 | 1.76 | 1.99 |
| METRNL | meteorin, glial cell differentiation regulator-like | 1.6 | 1.84 | 1.69 |
| ANXA3 | annexin A3 | 1.72 | 1.76 | 3.09 |
| GSN | gelsolin | 1.66 | 1.86 | 2.02 |
| LIPG | lipase, endothelial | −2.4 | −1.51 | −2.78 |
| PPIL1 | peptidylprolyl isomerase (cyclophilin)-like 1 | −1.79 | −1.62 | −1.71 |
| SYTL5 | synaptotagmin-like 5 | 1.93 | 1.92 | 2.55 |
| UPK3B | uroplakin 3B | 1.59 | 1.68 | 1.57 |
| SYNE1 | spectrin repeat containing, nuclear envelope 1 | 1.52 | 1.84 | 2.19 |
| PLSCR4 | phospholipid scramblase 4 | 2.32 | 1.73 | 2.95 |
| PTGER4 | prostaglandin E receptor 4 (subtype EP4) | 1.51 | 1.51 | 2.24 |
| GMFG | glia maturation factor, gamma | −2.24 | −2.02 | −2.32 |
| MAFF | v-maf musculoaponeurotic fibrosarcoma oncogene | −1.64 | −2.11 | −1.79 |
| | homolog F (avian) | | | |
| TMEM37 | transmembrane protein 37 | 1.87 | 2.34 | 2.75 |
| HCFC1R1 | host cell factor C1 regulator 1 (XPO1 dependent) | 1.6 | 1.66 | 1.97 |
| ZDHHC8P1 | zinc finger, DHHC-type containing 8 pseudogene 1 | 2.78 | 1.54 | 3.32 |
| AXL | AXL receptor tyrosine kinase | 1.73 | 1.68 | 2.53 |
| HLA-E | major histocompatibility complex, class I, E | 1.53 | 1.71 | 1.53 |
| MVK | mevalonate kinase | −1.86 | −1.5 | −2.19 |
| CASQ1 | calsequestrin 1 (fast-twitch, skeletal muscle) | 1.82 | 1.9 | 1.93 |
| EBP | emopamil binding protein (sterol isomerase) | −1.74 | −1.56 | −1.72 |
| DNAJC4 | DnaJ (Hsp40) homolog, subfamily C, member 4 | 1.62 | 1.71 | 1.8 |
| BTN3A3 | butyrophilin, subfamily 3, member A3 | 1.68 | 2 | 2.21 |
| LRMP | lymphoid-restricted membrane protein | 1.58 | 1.63 | 1.9 |
| IRF9 | interferon regulatory factor 9 | 1.65 | 1.62 | 1.88 |
| ART3 | ADP-ribosyltransferase 3 | −1.74 | −1.54 | −2.47 |
| LYAR | Ly1 antibody reactive homolog (mouse) | −1.75 | −1.62 | −1.76 |
| SNRPD1 | small nuclear ribonucleoprotein D1 polypeptide | −1.56 | −1.56 | −1.59 |
| | 16 kDa | | | |
| UPK2 | uroplakin 2 | 1.56 | 1.82 | 1.66 |
| MTHFD1L | methylenetetrahydrofolate dehydrogenase (NADP+ | −1.98 | −1.55 | −2.14 |
| | dependent) 1-like | | | |
| EGFL6 | EGF-like-domain, multiple 6 | 2.21 | 2.49 | 1.98 |
| BST2 | bone marrow stromal cell antigen 2 | 1.53 | 1.59 | 1.95 |
| LOC283788 | FSHD region gene 1 pseudogene | 1.69 | 2.05 | 2.07 |
| AGPAT5 | 1-acylglycerol-3-phosphate O-acyltransferase 5 | −1.63 | −1.73 | −1.52 |
| | (lysophosphatidic acid acyltransferase, epsilon) | | | |
| SERPINF1 | serpin peptidase inhibitor, clade F (alpha-2 | 1.52 | 1.52 | 1.92 |
| | antiplasmin, pigment epithelium derived factor), | | | |
| | member 1 | | | |
| CTSS | cathepsin S | 1.66 | 2.42 | 2.05 |
| PROS1 | protein S (alpha) | 1.98 | 2.11 | 2.43 |
| TFF1 | trefoil factor 1 | −1.59 | −1.77 | −2.86 |
| GJB2 | gap junction protein, beta 2, 26 kDa | −1.62 | −1.65 | −1.72 |
| TBC1D9 | TBC1 domain family, member 9 (with GRAM | 1.51 | 1.59 | 2.15 |
| | domain) | | | |
| C9orf40 | chromosome 9 open reading frame 40 | −1.67 | −1.73 | −1.58 |
| IPO5 | importin 5 | −2.87 | −1.52 | −1.73 |
| LOC100289610 | similar to mesenchymal stem cell protein DSC96 | −1.57 | −1.83 | −1.87 |
| GPC3 | glypican 3 | 1.92 | 1.62 | 1.79 |
| PDK4 | pyruvate dehydrogenase kinase, isozyme 4 | 2.12 | 2.61 | 3.55 |
| NFKBIA | nuclear factor of kappa light polypeptide gene | 1.74 | 1.57 | 1.67 |
| | enhancer in B-cells inhibitor, alpha | | | |
| CASZ1 | castor zinc finger 1 | 1.78 | 1.81 | 2.5 |
| SNCG | synuclein, gamma (breast cancer-specific protein 1) | 1.59 | 1.71 | 1.67 |
| TIPIN | TIMELESS interacting protein | −1.6 | −1.91 | −1.67 |
| EPHA4 | EPH receptor A4 | 1.59 | 1.85 | 1.99 |
| BAMBI | BMP and activin membrane-bound inhibitor | 1.56 | 2.44 | 1.52 |
| | homolog (Xenopus laevis) | | | |
| LMO4 | LIM domain only 4 | 1.66 | 1.63 | 2.31 |
| PIK3C3 | phosphoinositide-3-kinase, class 3 | 1.59 | 1.56 | 1.74 |
| CXCL11 | chemokine (C-X-C motif) ligand 11 | −1.62 | −1.69 | −3.2 |
| IL1R1 | interleukin 1 receptor, type I | 1.74 | 2.38 | 2.36 |
| HSD17B2 | hydroxysteroid (17-beta) dehydrogenase 2 | −1.92 | −1.53 | −1.52 |
| PEA15 | phosphoprotein enriched in astrocytes 15 | −1.55 | −1.61 | −1.56 |
| IRAK2 | interleukin-1 receptor-associated kinase 2 | −1.56 | −1.69 | −1.8 |
| PRODH | proline dehydrogenase (oxidase) 1 | 1.69 | 1.59 | 1.93 |
| CYP26B1 | cytochrome P450, family 26, subfamily B, | 1.55 | 1.61 | 1.84 |
| | polypeptide 1 | | | |
| WDR78 | WD repeat domain 78 | 1.97 | 2 | 2.73 |
| WLS | wntless homolog (Drosophila) | 1.51 | 1.79 | 2.8 |
| SGSH | N-sulfoglucosamine sulfohydrolase | 1.6 | 1.98 | 1.86 |
| KLF9 | Kruppel-like factor 9 | 1.55 | 2.11 | 1.99 |
| CHORDC1 | cysteine and histidine-rich domain (CHORD) | −1.72 | −1.7 | −1.86 |
| | containing 1 | | | |
| TRPC1 | transient receptor potential cation channel, | 1.88 | 1.81 | 1.86 |
| | subfamily C, member 1 | | | |
| HS6ST3 | heparan sulfate 6-O-sulfotransferase 3 | 2 | 2.02 | 1.62 |
| ETV5 | ets variant 5 | −1.88 | −2.36 | −2.16 |
| TRIM31 | tripartite motif containing 31 | 2.18 | 1.67 | 1.62 |
| COL4A1 | collagen, type IV, alpha 1 | −1.57 | −1.69 | −1.91 |
| C3orf26 | chromosome 3 open reading frame 26 | −1.71 | −1.52 | −1.72 |
| RPS6KA6 | ribosomal protein S6 kinase, 90 kDa, polypeptide 6 | 1.54 | 1.68 | 2.16 |
| BMP2 | bone morphogenetic protein 2 | 2.07 | 2.33 | 1.79 |
| SSFA2 | sperm specific antigen 2 | −1.89 | −1.94 | −2.2 |
| TMCC3 | transmembrane and coiled-coil domain family 3 | 1.57 | 2.41 | 2.29 |
| IL1RAP | interleukin 1 receptor accessory protein | −2.32 | −1.86 | −1.73 |
| BBOX1 | butyrobetaine (gamma), 2-oxoglutarate | 1.67 | 1.85 | 1.63 |
| | dioxygenase (gamma-butyrobetaine hydroxylase) 1 | | | |
| TMEM27 | transmembrane protein 27 | 1.59 | 1.64 | 2.67 |
| PDSS1 | prenyl (decaprenyl) diphosphate synthase, subunit 1 | −1.65 | −1.57 | −1.55 |
| DSE | dermatan sulfate epimerase | 1.71 | 1.91 | 1.89 |
| NR3C1 | nuclear receptor subfamily 3, group C, member 1 | 1.58 | 1.54 | 2.16 |
| | (glucocorticoid receptor) | | | |
| CPEB2 | cytoplasmic polyadenylation element binding | 2.01 | 2.52 | 3.08 |
| | protein 2 | | | |
| TPRG1 | tumor protein p63 regulated 1 | −1.75 | −1.82 | −1.75 |
| C15orf57 | chromosome 15 open reading frame 57 | 1.51 | 1.64 | 1.75 |
| MGAM | maltase-glucoamylase (alpha-glucosidase) | 1.83 | 1.87 | 2.29 |
| HAMP | hepcidin antimicrobial peptide | −1.57 | −1.83 | −1.7 |
| TLR4 | toll-like receptor 4 | −1.8 | −2.09 | −1.96 |
| GABRB3 | gamma-aminobutyric acid (GABA) A receptor, | 1.69 | 2.01 | 1.86 |
| | beta 3 | | | |
| GATA6 | GATA binding protein 6 | 1.59 | 1.99 | 2.41 |
| CLCN4 | chloride channel 4 | −2.05 | −1.94 | −2.07 |
| ZNF763 | zinc finger protein 763 | 1.56 | 1.63 | 2.8 |
| ACP1 | acid phosphatase 1, soluble | −1.51 | −1.56 | −1.55 |
| GIMAP2 | GTPase, IMAP family member 2 | 1.75 | 1.73 | 2.34 |
| LOC284837 | hypothetical LOC284837 | 1.55 | 1.69 | 1.63 |
| SNRPN | small nuclear ribonucleoprotein polypeptide N | 1.63 | 1.62 | 2.61 |
| MBD5 | methyl-CpG binding domain protein 5 | 1.84 | 1.52 | 1.65 |
| CD109 | CD109 molecule | 1.81 | 1.53 | 1.8 |
| JSRP1 | junctional sarcoplasmic reticulum protein 1 | −1.96 | −1.87 | −1.69 |
| TMEM151B | transmembrane protein 151B | −1.64 | −1.6 | −1.6 |
| PIWIL1 | piwi-like 1 (Drosophila) | −1.78 | −1.84 | −1.92 |
| FAM65B | family with sequence similarity 65, member B | 1.83 | 1.71 | 1.75 |
| EML5 | echinoderm microtubule associated protein like 5 | 1.68 | 1.75 | 1.93 |
| COL4A3 | collagen, type IV, alpha 3 (Goodpasture antigen) | 1.75 | 1.56 | 2.16 |
| PRKD2 | protein kinase D2 | −2.06 | −1.76 | −1.76 |
| MATR3 | matrin 3 | −1.85 | −3.05 | −1.84 |
| ACER3 | alkaline ceramidase 3 | −1.59 | −1.64 | −1.52 |
| NCRNA00247 | non-protein coding RNA 247 | 1.55 | 1.6 | 1.7 |
| LOC100507557 | hypothetical LOC100507557 | 1.58 | 1.74 | 2.02 |

Although the foregoing invention has been described in some detail by way of illustration and example for purposes of clarity of understanding, the descriptions and examples should not be construed as limiting the scope. The disclosures of all patent and scientific literature cited herein are expressly incorporated in their entirety by reference.

1. A method for treating an individual with disease or disorder comprising administering a therapeutically effective amount of a FGFR3 antagonist to the individual if the individual has been found to have elevated levels of a FGFR3 biomarker.

2. A method for treating a disease or disorder in an individual, the method comprising: determining that a sample from the individual comprises elevated levels of a FGFR3 biomarker, and administering an effective amount of a FGFR3 antagonist to the individual, whereby the disease or disorder is treated.

3-4. (canceled)

5. A method of identifying an individual with a disease or disorder who is more or less likely to exhibit benefit from treatment comprising a FGFR3 antagonist, the method comprising: determining levels of a FGFR3 biomarker in a sample from the individual, wherein elevated levels of the FGFR3 biomarker in the sample indicates that the individual is more likely to exhibit benefit from treatment comprising the FGFR3 antagonist or a reduced levels of the FGFR3 biomarker indicates that the individual is less likely to exhibit benefit from treatment comprising the FGFR3 antagonist.

6-8. (canceled)

9. The method of claim 1, wherein the method further comprises administering an effective amount of the FGFR3 antagonist to the individual.

10. The method, assay and/or kit of claim 1, wherein the FGFR3 biomarker is FGFR3.

11. The method, assay and/or kit of claim 10, wherein FGFR3 is detected by immunohistochemistry.

12. The method, assay and/or kit of claim 10, wherein FGFR3 is detected by immunohistochemistry using sc-13121 from Santa Cruz Biotechnology.

13. The method, assay and/or kit of claim 1, wherein elevated levels of a FGFR3 biomarker is detected by IHC clinical diagnosis of positive or IHC clinical score of 1 or higher.

14. The method, assay and/or kit of claim 13, wherein the IHC clinical score of 1 or higher is 2 or higher.

15. The method, assay and/or kit of claim 13, wherein the IHC clinical score of 1 or higher is 3.

16. The method, assay and/or kit of claim 1, wherein the sample is a tissue sample.

17. The method, assay and/or kit of claim 16, wherein the tissue sample is a tumor tissue sample.

18. The method, assay and/or kit of claim 1, wherein the FGFR3 antagonist is an antibody, binding polypeptide, small molecule, and/or polynucleotide.

19. The method, assay and/or kit of claim 18, wherein the FGFR3 antagonist is an anti-FGFR3 antibody.

20. The method, assay and/or kit of claim 19, wherein the antibody is a monoclonal antibody.

21. The method, assay and/or kit of claim 19, wherein the antibody is a human, humanized, or chimeric antibody.

22. The method, assay and/or kit of claim 1, wherein the disease or disorder is a proliferative disease or disorder.

23. The method, assay and/or kit of claim 22, wherein the proliferative disease or disorder is cancer.


SEQUENCE LISTING (182 sequences)

SEQ ID NO: 1 - protein, 10 aa, Artificial Sequence
  Description of Artificial Sequence: Synthetic peptide
GFTFTSTGIS

SEQ ID NO: 2 - protein, 18 aa, Artificial Sequence
  Description of Artificial Sequence: Synthetic peptide
GRIYPTNGSTNYADSVKG

SEQ ID NO: 3 - protein, 20 aa, Artificial Sequence
  Description of Artificial Sequence: Synthetic peptide
ARTYGIYDLYVDYTEYVMDY

SEQ ID NO: 4 - protein, 11 aa, Artificial Sequence
  Description of Artificial Sequence: Synthetic peptide
RASQDVSTAVA

SEQ ID NO: 5 - protein, 7 aa, Artificial Sequence
  Description of Artificial Sequence: Synthetic peptide
SASFLYS

SEQ ID NO: 6 - protein, 9 aa, Artificial Sequence
  Description of Artificial Sequence: Synthetic peptide
QQSYTTPPT

SEQ ID NO: 7 - protein, 10 aa, Artificial Sequence
  Description of Artificial Sequence: Synthetic peptide
GFTFTSTGIS

SEQ ID NO: 8 - protein, 18 aa, Artificial Sequence
  Description of Artificial Sequence: Synthetic peptide
GRIYPTNGSTNYADSVKG

SEQ ID NO: 9 - protein, 20 aa, Artificial Sequence
  Description of Artificial Sequence: Synthetic peptide
ARTYGIYDLYVDYTEYVMDY

SEQ ID NO: 10 - protein, 11 aa, Artificial Sequence
  Description of Artificial Sequence: Synthetic peptide
RASQDVDTSLA

SEQ ID NO: 11 - protein, 7 aa, Artificial Sequence
  Description of Artificial Sequence: Synthetic peptide
SASFLYS

SEQ ID NO: 12 - protein, 9 aa, Artificial Sequence
  Description of Artificial Sequence: Synthetic peptide
QQSTGHPQT

SEQ ID NO: 13 - protein, 10 aa, Artificial Sequence
  Description of Artificial Sequence: Synthetic peptide
GFTFTSTGIS

SEQ ID NO: 14 - protein, 18 aa, Artificial Sequence
  Description of Artificial Sequence: Synthetic peptide
GRIYPTNGSTNYADSVKG

SEQ ID NO: 15 - protein, 20 aa, Artificial Sequence
  Description of Artificial Sequence: Synthetic peptide
ARTYGIYDLYVDYTEYVMDY

SEQ ID NO: 16 - protein, 11 aa, Artificial Sequence
  Description of Artificial Sequence: Synthetic peptide
RASQDVDISLA

SEQ ID NO: 17 - protein, 7 aa, Artificial Sequence
  Description of Artificial Sequence: Synthetic peptide
SASSLAS

SEQ ID NO: 18 - protein, 9 aa, Artificial Sequence
  Description of Artificial Sequence: Synthetic peptide
QQGAGNPYT

SEQ ID NO: 19-47 - skipped (29 entries)

SEQ ID NO: 48 - protein, 10 aa, Artificial Sequence
  Description of Artificial Sequence: Synthetic peptide
GFTFSTTGIS

SEQ ID NO: 49 - protein, 18 aa, Artificial Sequence
  Description of Artificial Sequence: Synthetic peptide
GRIYPLYGSTHYADSVKG

SEQ ID NO: 50 - protein, 20 aa, Artificial Sequence
  Description of Artificial Sequence: Synthetic peptide
ARTYGIYDLYVDYTEYVMDY

SEQ ID NO: 51 - protein, 11 aa, Artificial Sequence
  Description of Artificial Sequence: Synthetic peptide
RASQDVSTAVA

SEQ ID NO: 52 - protein, 7 aa, Artificial Sequence
  Description of Artificial Sequence: Synthetic peptide
SASFLYS

SEQ ID NO: 53 - protein, 9 aa, Artificial Sequence
  Description of Artificial Sequence: Synthetic peptide
QQTYTTSLT

SEQ ID NO: 54 - protein, 10 aa, Artificial Sequence
  Description of Artificial Sequence: Synthetic peptide
GFTFTSTGIS

SEQ ID NO: 55 - protein, 18 aa, Artificial Sequence
  Description of Artificial Sequence: Synthetic peptide
GRIYPYDDSFYYADSVKG

SEQ ID NO: 56 - protein, 20 aa, Artificial Sequence
  Description of Artificial Sequence: Synthetic peptide
ARTYGIYDLYVDYTEYVMDY

SEQ ID NO: 57 - protein, 11 aa, Artificial Sequence
  Description of Artificial Sequence: Synthetic peptide
RASQDVSTAVA

SEQ ID NO: 58 - protein, 7 aa, Artificial Sequence
  Description of Artificial Sequence: Synthetic peptide
SASFLYS

SEQ ID NO: 59 - protein, 9 aa, Artificial Sequence
  Description of Artificial Sequence: Synthetic peptide
QQSYTTPLT

SEQ ID NO: 60 - protein, 10 aa, Artificial Sequence
  Description of Artificial Sequence: Synthetic peptide
GFTFTSTGIS

SEQ ID NO: 61 - protein, 18 aa, Artificial Sequence
  Description of Artificial Sequence: Synthetic peptide
GRIYPTNGSTNYADSVKG

SEQ ID NO: 62 - protein, 20 aa, Artificial Sequence
  Description of Artificial Sequence: Synthetic peptide
ARTYGIYDLYVDYTEYVMDY

SEQ ID NO: 63 - protein, 11 aa, Artificial Sequence
  Description of Artificial Sequence: Synthetic peptide
RASQVIDISLA

SEQ ID NO: 64 - protein, 7 aa, Artificial Sequence
  Description of Artificial Sequence: Synthetic peptide
GASTLAS

SEQ ID NO: 65 - protein, 9 aa, Artificial Sequence
  Description of Artificial Sequence: Synthetic peptide
QQSAADPYT

SEQ ID NO: 66 - protein, 10 aa, Artificial Sequence
  Description of Artificial Sequence: Synthetic peptide
GFSFTGTGIS

SEQ ID NO: 67 - protein, 18 aa, Artificial Sequence
  Description of Artificial Sequence: Synthetic peptide
GSIYPYFATKNYADSVKG

SEQ ID NO: 68 - protein, 20 aa, Artificial Sequence
  Description of Artificial Sequence: Synthetic peptide
ARTYGIYDLYVDYTEYVMDY

SEQ ID NO: 69 - protein, 11 aa, Artificial Sequence
  Description of Artificial Sequence: Synthetic peptide
RASQDVSTAVA

SEQ ID NO: 70 - protein, 7 aa, Artificial Sequence
  Description of Artificial Sequence: Synthetic peptide
SASFLYS

SEQ ID NO: 71 - protein, 9 aa, Artificial Sequence
  Description of Artificial Sequence: Synthetic peptide
QQSYTTPPT

SEQ ID NO: 72 - protein, 10 aa, Artificial Sequence
  Description of Artificial Sequence: Synthetic peptide
GFTFYTTGIS

SEQ ID NO: 73 - protein, 18 aa, Artificial Sequence
  Description of Artificial Sequence: Synthetic peptide
GRIYPAFGSSIYADSVKG

SEQ ID NO: 74 - protein, 20 aa, Artificial Sequence
  Description of Artificial Sequence: Synthetic peptide
ARTYGIYDLYVDYTEYVMDY

SEQ ID NO: 75 - protein, 11 aa, Artificial Sequence
  Description of Artificial Sequence: Synthetic peptide
RASQDVSTAVA

SEQ ID NO: 76 - protein, 7 aa, Artificial Sequence
  Description of Artificial Sequence: Synthetic peptide
SASFLYS

SEQ ID NO: 77 - protein, 9 aa, Artificial Sequence
  Description of Artificial Sequence: Synthetic peptide
QQTYSAQPT

SEQ ID NO: 78 - protein, 10 aa, Artificial Sequence
  Description of Artificial Sequence: Synthetic peptide
GFSFWSTGIS

SEQ ID NO: 79 - protein, 18 aa, Artificial Sequence
  Description of Artificial Sequence: Synthetic peptide
GRIYPSSATTNYADSVKG

SEQ ID NO: 80 - protein, 20 aa, Artificial Sequence
  Description of Artificial Sequence: Synthetic peptide
ARTYGIYDLYVDYTEYVMDY

SEQ ID NO: 81 - protein, 11 aa, Artificial Sequence
  Description of Artificial Sequence: Synthetic peptide
RASQDVSTAVA

SEQ ID NO: 82 - protein, 7 aa, Artificial Sequence
  Description of Artificial Sequence: Synthetic peptide
SASFLYS

SEQ ID NO: 83 - protein, 9 aa, Artificial Sequence
  Description of Artificial Sequence: Synthetic peptide
QQSYSHQST

SEQ ID NO: 84 - protein, 10 aa, Artificial Sequence
  Description of Artificial Sequence: Synthetic peptide
GFTFTSTGIS

SEQ ID NO: 85 - protein, 18 aa, Artificial Sequence
  Description of Artificial Sequence: Synthetic peptide
GRIYPTSGSTNYADSVKG

SEQ ID NO: 86 - protein, 20 aa, Artificial Sequence
  Description of Artificial Sequence: Synthetic peptide
ARTYGIYDLYVDYTEYVMDY

SEQ ID NO: 87 - protein, 11 aa, Artificial Sequence
  Description of Artificial Sequence: Synthetic peptide
RASQDVDTSLA

SEQ ID NO: 88 - protein, 7 aa, Artificial Sequence
  Description of Artificial Sequence: Synthetic peptide
SASFLYS

SEQ ID NO: 89 - protein, 9 aa, Artificial Sequence
  Description of Artificial Sequence: Synthetic peptide
QQSTGHPQT

SEQ ID NO: 90 - protein, 10 aa, Artificial Sequence
  Description of Artificial Sequence: Synthetic peptide
GFTFTSTGIS

SEQ ID NO: 91 - protein, 18 aa, Artificial Sequence
  Description of Artificial Sequence: Synthetic peptide
GRIYPTGGSTNYADSVKG

SEQ ID NO: 92 - protein, 20 aa, Artificial Sequence
  Description of Artificial Sequence: Synthetic peptide
ARTYGIYDLYVDYTEYVMDY

SEQ ID NO: 93 - protein, 11 aa, Artificial Sequence
  Description of Artificial Sequence: Synthetic peptide
RASQDVDTSLA

SEQ ID NO: 94 - protein, 7 aa, Artificial Sequence
  Description of Artificial Sequence: Synthetic peptide
SASFLYS

SEQ ID NO: 95 - protein, 9 aa, Artificial Sequence
  Description of Artificial Sequence: Synthetic peptide
QQSTGHPQT

SEQ ID NO: 96 - protein, 10 aa, Artificial Sequence
  Description of Artificial Sequence: Synthetic peptide
GFTFTSTGIS

SEQ ID NO: 97 - protein, 18 aa, Artificial Sequence
  Description of Artificial Sequence: Synthetic peptide
GRIYPTAGSTNYADSVKG

SEQ ID NO: 98 - protein, 20 aa, Artificial Sequence
  Description of Artificial Sequence: Synthetic peptide
ARTYGIYDLYVDYTEYVMDY

SEQ ID NO: 99 - protein, 11 aa, Artificial Sequence
  Description of Artificial Sequence: Synthetic peptide
RASQDVDTSLA

SEQ ID NO: 100 - protein, 7 aa, Artificial Sequence
  Description of Artificial Sequence: Synthetic peptide
SASFLYS

SEQ ID NO: 101 - protein, 9 aa, Artificial Sequence
  Description of Artificial Sequence: Synthetic peptide
QQSTGHPQT

SEQ ID NO: 102 - protein, 10 aa, Artificial Sequence
  Description of Artificial Sequence: Synthetic peptide
GFTFTSTGIS

SEQ ID NO: 103 - protein, 18 aa, Artificial Sequence
  Description of Artificial Sequence: Synthetic peptide
GRIYPTQGSTNYADSVKG

SEQ ID NO: 104 - protein, 20 aa, Artificial Sequence
  Description of Artificial Sequence: Synthetic peptide
ARTYGIYDLYVDYTEYVMDY

SEQ ID NO: 105 - protein, 11 aa, Artificial Sequence
  Description of Artificial Sequence: Synthetic peptide
RASQDVDTSLA

SEQ ID NO: 106 - protein, 7 aa, Artificial Sequence
  Description of Artificial Sequence: Synthetic peptide
SASFLYS

SEQ ID NO: 107 - protein, 9 aa, Artificial Sequence
  Description of Artificial Sequence: Synthetic peptide
QQSTGHPQT

SEQ ID NO: 108 - protein, 10 aa, Artificial Sequence
  Description of Artificial Sequence: Synthetic peptide
GFTFTSTGIS

SEQ ID NO: 109 - protein, 18 aa, Artificial Sequence
  Description of Artificial Sequence: Synthetic peptide
GRIYPTSGSTNYADSVKG

SEQ ID NO: 110 - protein, 20 aa, Artificial Sequence
  Description of Artificial Sequence: Synthetic peptide
ARTYGIYDLYVDYTEYVMDY

SEQ ID NO: 111 - protein, 11 aa, Artificial Sequence
  Description of Artificial Sequence: Synthetic peptide
RASQVVDTSLA

SEQ ID NO: 112 - protein, 7 aa, Artificial Sequence
  Description of Artificial Sequence: Synthetic peptide
SASSLAS

SEQ ID NO: 113 - protein, 9 aa, Artificial Sequence
  Description of Artificial Sequence: Synthetic peptide
QQGAGNPYT

SEQ ID NO: 114 - protein, 10 aa, Artificial Sequence
  Description of Artificial Sequence: Synthetic peptide
GFTFTSTGIS

SEQ ID NO: 115 - protein, 18 aa, Artificial Sequence
  Description of Artificial Sequence: Synthetic peptide
GRIYPTGGSTNYADSVKG

SEQ ID NO: 116 - protein, 20 aa, Artificial Sequence
  Description of Artificial Sequence: Synthetic peptide
ARTYGIYDLYVDYTEYVMDY

SEQ ID NO: 117 - protein, 11 aa, Artificial Sequence
  Description of Artificial Sequence: Synthetic peptide
RASQVVDTSLA

SEQ ID NO: 118 - protein, 7 aa, Artificial Sequence
  Description of Artificial Sequence: Synthetic peptide
SASSLAS

SEQ ID NO: 119 - protein, 9 aa, Artificial Sequence
  Description of Artificial Sequence: Synthetic peptide
QQGAGNPYT

SEQ ID NO: 120 - protein, 10 aa, Artificial Sequence
  Description of Artificial Sequence: Synthetic peptide
GFTFTSTGIS

SEQ ID NO: 121 - protein, 18 aa, Artificial Sequence
  Description of Artificial Sequence: Synthetic peptide
GRIYPTAGSTNYADSVKG

SEQ ID NO: 122 - protein, 20 aa, Artificial Sequence
  Description of Artificial Sequence: Synthetic peptide
ARTYGIYDLYVDYTEYVMDY

SEQ ID NO: 123 - protein, 11 aa, Artificial Sequence
  Description of Artificial Sequence: Synthetic peptide
RASQVVDTSLA

SEQ ID NO: 124 - protein, 7 aa, Artificial Sequence
  Description of Artificial Sequence: Synthetic peptide
SASSLAS

SEQ ID NO: 125 - protein, 9 aa, Artificial Sequence
  Description of Artificial Sequence: Synthetic peptide
QQGAGNPYT

SEQ ID NO: 126 - protein, 10 aa, Artificial Sequence
  Description of Artificial Sequence: Synthetic peptide
GFTFTSTGIS

SEQ ID NO: 127 - protein, 18 aa, Artificial Sequence
  Description of Artificial Sequence: Synthetic peptide
GRIYPTQGSTNYADSVKG

SEQ ID NO: 128 - protein, 20 aa, Artificial Sequence
  Description of Artificial Sequence: Synthetic peptide
ARTYGIYDLYVDYTEYVMDY

SEQ ID NO: 129 - protein, 11 aa, Artificial Sequence
  Description of Artificial Sequence: Synthetic peptide
RASQVVDTSLA

SEQ ID NO: 130 - protein, 7 aa, Artificial Sequence
  Description of Artificial Sequence: Synthetic peptide
SASSLAS

SEQ ID NO: 131 - protein, 9 aa, Artificial Sequence
  Description of Artificial Sequence: Synthetic peptide
QQGAGNPYT

SEQ ID NO: 132 - protein, 125 aa, Artificial Sequence
  Description of Artificial Sequence: Synthetic polypeptide
EVQLVESGGGLVQPGGSLRLSCAASGFTFTSTGISWVRQAPGKGLEWVGRIYPTSGSTNYADSVKGRFTISADTSKNTAYLQMNSLRAEDTAVYYCARARTYGIYDLYVDYTEYVMDYWGQGTLV

SEQ ID NO: 133 - protein, 108 aa, Artificial Sequence
  Description of Artificial Sequence: Synthetic polypeptide
DIQMTQSPSSLSASVGDRVTITCRASQDVDTSLAWYQQKPGKAPKLLIYSASFLYSGVPSRFSGSGSGTDFTLTISSLQPEDFATYYCQQSTGHPQTFGQGTKVEIKR

SEQ ID NO: 134 - protein, 123 aa, Artificial Sequence
  Description of Artificial Sequence: Synthetic polypeptide
EVQLVESGGGLVQPGGSLRLSCAASGFTFTSTGISWVRQAPGKGLEWVGRIYPTSGSTNYADSVKGRFTISADTSKNTAYLQMNSLRAEDTAVYYCARTYGIYDLYVDYTEYVMDYWGQGTLV

SEQ ID NO: 135 - protein, 108 aa, Artificial Sequence
  Description of Artificial Sequence: Synthetic polypeptide
DIQMTQSPSSLSASVGDRVTITCRASQDVDTSLAWYKQKPGKAPKLLIYSASFLYSGVPSRFSGSGSGTDFTLTISSLQPEDFATYYCQQSTGHPQTFGQGTKVEIKR

SEQ ID NO: 136 - protein, 125 aa, Artificial Sequence
  Description of Artificial Sequence: Synthetic polypeptide
EVQLVESGGGLVQPGGSLRLSCAASGFTFTSTGISWVRQAPGKGLEWVARIYPTNGSTNYADSVKGRFTISADTSKNTAYLQMNSLRAEDTAVYYCARARTYGIYDLYVDYTEYVMDYWGQGTLV

SEQ ID NO: 137 - protein, 108 aa, Artificial Sequence
  Description of Artificial Sequence: Synthetic polypeptide
DIQMTQSPSSLSASVGDRVTITCRASQDVDISLAWYQQKPGKAPKLLIYSASFLYSGVPSRFSGSGSGTDFTLTISSLQPEDFATYYCQQGAGNPYTFGQGTKVEIKR

SEQ ID NO: 138 - protein, 125 aa, Artificial Sequence
  Description of Artificial Sequence: Synthetic polypeptide
EVQLVESGGGLVQPGGSLRLSCAASGFTFSTTGISWVRQAPGKGLEWVARIYPLYGSTHYADSVKGRFTISADTSKNTAYLQMNSLRAEDTAVYYCARARTYGIYDLYVDYTEYVMDYWGQGTLV

SEQ ID NO: 139 - protein, 108 aa, Artificial Sequence
  Description of Artificial Sequence: Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQTYTTSLTFGQGTKVEIKR

SEQ ID NO: 140 - protein, 11 aa, Artificial Sequence
  Description of Artificial Sequence: Synthetic peptide
RASQDVETSLA

SEQ ID NO: 141 - protein, 7 aa, Artificial Sequence
  Description of Artificial Sequence: Synthetic peptide
SASFLYS

SEQ ID NO: 142 - protein, 9 aa, Artificial Sequence
  Description of Artificial Sequence: Synthetic peptide
QQSTGHPQT

SEQ ID NO: 143 - protein, 10 aa, Artificial Sequence
  Description of Artificial Sequence: Synthetic peptide
GFTFTSTGIS

SEQ ID NO: 144 - protein, 18 aa, Artificial Sequence
  Description of Artificial Sequence: Synthetic peptide
GRIYPTSGSTNYADSVKG

SEQ ID NO: 145 - protein, 20 aa, Artificial Sequence
  Description of Artificial Sequence: Synthetic peptide
ARTYGIYDLYVDYTEYVMDY

SEQ ID NO: 146 - protein, 11 aa, Artificial Sequence
  Description of Artificial Sequence: Synthetic peptide
  MOD_RES (5)..(5): Val or Asp
  MOD_RES (6)..(6): Val or Ile
  MOD_RES (7)..(7): Asp, Glu or Ser
  MOD_RES (8)..(8): Thr or Ile
  MOD_RES (9)..(9): Ala or Ser
  MOD_RES (10)..(10): Val or Leu
RASQXXXXXXA

SEQ ID NO: 147 - protein, 7 aa, Artificial Sequence
  Description of Artificial Sequence: Synthetic peptide
  MOD_RES (1)..(1): Ser or Gly
  MOD_RES (6)..(6): Ala or Tyr
XASFLXS

SEQ ID NO: 148 - protein, 9 aa, Artificial Sequence
  Description of Artificial Sequence: Synthetic peptide
  MOD_RES (3)..(3): Gly, Ser or Thr
  MOD_RES (4)..(4): Thr, Tyr or Ala
  MOD_RES (5)..(5): Gly, Ser, Thr or Ala
  MOD_RES (6)..(6): Ala, His, Asp, Thr or Asn
  MOD_RES (7)..(7): Gln, Pro or Ser
  MOD_RES (8)..(8): Ser, Tyr, Leu, Pro or Gln
QQXXXXXXT

SEQ ID NO: 149 - protein, 10 aa, Artificial Sequence
  Description of Artificial Sequence: Synthetic peptide
  MOD_RES (3)..(3): Ser or Thr
  MOD_RES (5)..(5): Trp, Tyr, Ser or Thr
  MOD_RES (6)..(6): Ser, Gly or Thr
GFXFXXTGIS

SEQ ID NO: 150 - protein, 18 aa, Artificial Sequence
  Description of Artificial Sequence: Synthetic peptide
  MOD_RES (6)..(6): Tyr, Ala, Leu, Ser or Thr
  MOD_RES (7)..(7): Ala, Gln, Asp, Gly, Tyr, Ser, Asn or Phe
  MOD_RES (8)..(8): Ala, Asp or Gly
  MOD_RES (9)..(9): Thr or Ser
  MOD_RES (10)..(10): Lys, Phe, Thr or Ser
  MOD_RES (11)..(11): Tyr, His, Asn or Ile
GRIYPXXXXXXYADSVKG

SEQ ID NO: 151 - protein, 20 aa, Artificial Sequence
  Description of Artificial Sequence: Synthetic peptide
ARTYGIYDLYVDYTEYVMDY

SEQ ID NO: 152 - protein, 11 aa, Artificial Sequence
  Description of Artificial Sequence: Synthetic peptide
  MOD_RES (5)..(5): Val or Asp
  MOD_RES (7)..(7): Asp, Glu or Ser
  MOD_RES (8)..(8): Thr or Ile
  MOD_RES (9)..(9): Ala or Ser
RASQXVXXXVA

SEQ ID NO: 153 - protein, 9 aa, Artificial Sequence
  Description of Artificial Sequence: Synthetic peptide
  MOD_RES (3)..(3): Ser, Gly or Thr
  MOD_RES (4)..(4): Tyr, Thr or Ala
  MOD_RES (5)..(5): Thr or Gly
  MOD_RES (6)..(6): Thr, His or Asn
  MOD_RES (7)..(7): Pro or Ser
  MOD_RES (8)..(8): Pro, Gln, Tyr or Leu
QQXXXXXXT

SEQ ID NO: 154 - protein, 18 aa, Artificial Sequence
  Description of Artificial Sequence: Synthetic peptide
  MOD_RES (6)..(6): Thr or Leu
  MOD_RES (7)..(7): Asn, Tyr, Ser, Gly, Ala or Gln
  MOD_RES (11)..(11): Asn or His
GRIYPXXGSTXYADSVKG

SEQ ID NO: 155 - protein, 10 aa, Mus musculus
SASSSVSYMH

SEQ ID NO: 156 - protein, 10 aa, Mus musculus
SASSSVSYMH

SEQ ID NO: 157 - protein, 11 aa, Mus musculus
LASQTIGTWLA

SEQ ID NO: 158 - protein, 11 aa, Mus musculus
TWIYDTSILAS

SEQ ID NO: 159 - protein, 11 aa, Mus musculus
RWIYDTSKLAS

SEQ ID NO: 160 - protein, 11 aa, Mus musculus
LLIYAATSLAD

SEQ ID NO: 161 - protein, 9 aa, Mus musculus
QQWTSNPLT

SEQ ID NO: 162 - protein, 9 aa, Mus musculus
QQWSSYPPT

SEQ ID NO: 163 - protein, 9 aa, Mus musculus
QQLYSPPWT

SEQ ID NO: 164 - protein, 10 aa, Mus musculus
GYSFTDYNMY

SEQ ID NO: 165 - protein, 10 aa, Mus musculus
GYVFTHYNMY

SEQ ID NO: 166 - protein, 10 aa, Mus musculus
GYAFTSYNMY

SEQ ID NO: 167 - protein, 19 aa, Mus musculus
IGYIEPYNGGTSYNQKFKG

SEQ ID NO: 168 - protein, 20 aa, Mus musculus
WIGYIEPYNGGTSYNQKFKG

SEQ ID NO: 169 - protein, 20 aa, Mus musculus
WIGYIDPYIGGTSYNQKFKG

SEQ ID NO: 170 - protein, 13 aa, Mus musculus
ASPNYYDSSPFAY

SEQ ID NO: 171 - protein, 10 aa, Mus musculus
ARGQGPDFDV

SEQ ID NO: 172 - protein, 13 aa, Mus musculus
ARWGDYDVGAMDY

SEQ ID NO: 173 - protein, 107 aa, Artificial Sequence [sequence omitted]

SEQ ID NO: 174 - protein, 107 aa, Artificial Sequence
  Description of Artificial Sequence: Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQSYTTPPTFGQGTKVEIK

SEQ ID NO: 175-178 - skipped (4 entries)

SEQ ID NO: 179 - protein, 15 aa, Homo sapiens
LAVPAANTVRFRCPA

SEQ ID NO: 180 - protein, 15 aa, Homo sapiens
SDVEFHCKVYSDAQP

SEQ ID NO: 181 - protein, 100 aa, Homo sapiens
LGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVR

SEQ ID NO: 182 - protein, 4 aa, Artificial Sequence
  Description of Artificial Sequence: Synthetic peptide
DEAD